(19) United States
(12) Patent Application Publication
(10) Pub. No.: US 2006/0040944 A1
(43) Pub. Date: Feb. 23, 2006

(54) 5-Aza-7-deazapurine derivatives for treating Flaviviridae

(76) Inventors: Gilles Gosselin, Montpellier (FR); Frank Seela, Osnabruck (DE); Paolo La Colla, Cagliari (IT); Richard Storer, Folkestone (GB); David Dukhan, Montpellier (FR); Frederic Leroy, Jonquieres (FR)

Correspondence Address: KING & SPALDING LLP, 191 PEACHTREE STREET, N.E., 45TH FLOOR, ATLANTA, GA, 30303-1763, US

(21) Appl. No.: 11/166,498

(22) Filed: Jun. 23, 2005

Related U.S. Application Data
(60) Provisional application No. 60/582,182, filed on Jun. 23, 2004.

(51) Int. Cl. A61K 31/53; C07D 487/04

(52) U.S. Cl. 514/243; 544/184

(57) ABSTRACT

This invention is directed to a method for treating a host, especially a human, infected with hepatitis C, flavivirus and/or pestivirus, comprising administering to that host an effective amount of an anti-flavivirus or anti-pestivirus, biologically active compound has a 5-aza-7-deazapurine moiety. The 5-aza-7-deazapurine moiety may be substituted or unsubstituted, and may comprise a nucleoside analogue, or a salt or prodrug thereof. The compound of the present invention may be administered alone or in combination with another anti-hepatitis C, anti-flavivirus and/or anti-pestivirus agent.

### CROSS-REFERENCE TO RELATED APPLICATIONS

This application claims priority to U.S. Provisional Application No. 60/582,182 filed Jun. 23, 2004.

### FIELD OF THE INVENTION

The present invention is in the area of pharmaceutical chemistry and provides nucleoside derivatives that have a non-natural purine-like base, their synthesis and their use as anti-Flaviviridae agents in the treatment of hosts infected with Flaviviridae.

### BACKGROUND OF THE INVENTION

The family of Flaviviridae viruses include pestiviruses, flaviviruses and hepatitis C virus. The pestivirus genus includes bovine viral diarrhea virus (BVDV), classical swine fever virus (CSFV, also known as hog cholera virus), and Border disease virus (BDV) of sheep (Moennig et al., Adv. Vir. Res. 1992, 41:53-98). Pestivirus infections of domesticated livestock (i.e., cattle, pigs, and sheep) cause significant economic losses worldwide. BVDV causes mucosal disease in cattle and is of significant economic importance to the livestock industry (Meyers, G. and Thiel, H-J., Adv. In Viral Res., 1996, 47:53-118; Moennig et al., Adv. Vir. Res. 1992, 41:53-98).

Human pestiviruses have not been as extensively characterized as animal pestiviruses. However, serological surveys indicate considerable pestivirus exposure in humans. Pestivirus infections in man have been implicated in several diseases including congenital brain injury, infantile gastroenteritis, and chronic diarrhea in human immunodeficiency virus (HIV) positive patients (M. Giangaspero et al., Arch. Virol. Suppl., 1993, 7:53-62; M. Giangaspero et al., Int. J. Std. Aids, 1993, 4(5):300-302).

The flavivirus genus includes more than 68 members that are separated into groups on the basis of serological relatedness (Calisher et al., J. Gen. Virol., 1993, 70:37-43). Clinical symptoms vary and include fever, encephalitis and hemorrhagic fever (Fields Virology, Ed.: Fields, B. N., Knipe, D. M., and Howley, P. M.; Lippincott-Raven Publishers, Philadelphia, Pa.; 1996; Chapter 31, pp. 931-59). Flaviviruses of global concern that are associated with human disease include the dengue hemorrhagic fever virus (DHF or DENV), yellow fever virus (YFV), West Nile virus (WNV), shock syndrome and Japanese encephalitis virus (S. B. Halstead, Rev. Infect. Dis., 1984, 6:251-64; S. B. Halstead, Science, 1988, 239:476-81; T. P. Monath, New Engl. J. Med., 1988, 319:641-3).

Another flavivirus, hepatitis C virus (HCV), is the leading cause of chronic liver disease worldwide (N. Boyer et al., J. Hepatol. 2000, 32:98-112). HCV causes a slow-growing viral infection and is the major cause of cirrhosis and hepatocellular carcinoma (DiBesceglie, A. M. and B. R. Bacon, Scientific American, 1999, October:80-85; N. Boyer et al., J. Hepatol. 2000, 32:98-112). About 20% of those infected clear the virus, but the remainder harbor it for life. An estimated 170 million people are infected with HCV worldwide, and about 4.5 million in the United States alone (N. Boyer et al., J. Hepatol. 2000, 32:98-112). Cirrhosis caused by chronic HCV infection occurs in 10-20% of people infected, and accounts for 8-12,000 deaths per year in the United States, and HCV infection is the leading indication for liver transplant.

HCV is known to cause at least 80% of post-transfusion hepatitis and a substantial proportion of sporadic acute hepatitis. Preliminary evidence implicates HCV in many cases of “idiopathic” chronic hepatitis, “cryptogenic” cirrhosis, and probably hepatocellular carcinoma unrelated to other hepatitis viruses. A small proportion of healthy persons appear to be chronic HCV carriers, but this varies geographically and epidemiologically. The numbers may substantially exceed those for HBV although this information is still preliminary, and it is still unclear how many of these people have subclinical chronic liver disease (The Merck Manual, 1992, 16^{th} Ed., Chpt. 69, p. 901).

HCV is an enveloped virus containing a positive-sense single-stranded RNA genome of approximately 9.4 k. The viral genome consists of a 5′-untranslated region (UTR), a long open reading frame (ORF) encoding a polyprotein precursor of approximately 3011 amino acids, and a short 3′-UTR. The 5′-UTR is the most highly conserved part of the HCV genome and is important for the initiation and control of polyprotein translation. Translation of the HCV genome is initiated by a cap-independent mechanism known as internal ribosome entry. This mechanism involves the binding of ribosomes to an RNA sequence known as the internal ribosome entry site (IRES). An RNA pseudoknot structure has recently been determined to be an essential structural element of the HCV IRES. Viral structural proteins include a nucleocapsid core protein (C) and two envelope glycoproteins, E1 and E2. HCV also encodes two proteinases, a zinc-dependent metalloproteinase encoded by the NS2-NS3 region, and a serine proteinase encoded in the NS3 region. These proteinases are required for cleavage of specific regions of the precursor polyprotein into mature peptides. The carboxyl half of nonstructural protein 5, NS5, contains the RNA-dependent RNA polymerase. The function(s) of the remaining non-structural proteins, NS4A, NS4, and NS5A (the amino terminal half of non-structural protein 5) are the subjects of ongoing studies. The non-structural protein NS4A appears to be a serine protease (Hsu et al., Nat. Biotechnol., Apr. 23, 2003; [retrieved on Apr. 23, 2003]; retrieved from Entrez PubMed, Internet URL: http://www.ncbi.nlm.nih.gov/Entrez/), while studies on NS4 suggest its involvement in translational inhibition and consequent degradation of host cellular proteins (Forese et al., Virus Res., December 2002, 90(1-2):119-31). The non-structural protein NS5A has been shown to inhibit p53 activity on a p21 promoter region via its ability to bind to a specific DNA sequence, thereby blocking p53 activity (Gong et al., Zonghua Gan Zang Bing Za Zhi, March 2003, 11(3):162-5). Both NS3 and NS5A have been shown to be involved with host cellular signaling transduction pathways (Giannini et al., Cell Death Diff:, January 2003, 10 Suppl. 1:S27-28).

Idenix Pharmaceuticals, Ltd. discloses branched nucleosides, and their use in the treatment of HCV and flaviviruses and pestiviruses in U.S. Pat. No. 6.914,054, which will issue on Jul. 5, 2005, and U.S. Pat. No. 6,812,219, issued Nov. 2, 2004, which correspond to International Publication Nos. WO 01/90121 and WO 01/92282. A method for the treatment of hepatitis C infection (and flaviviruses and pestiviruses) in humans and other host animals is disclosed in the Idenix publications that includes administering an effective amount of a biologically active 1′, 2′, 3′ or 4′-branched β-D or β-L nucleosides or a pharmaceutically acceptable salt or prodrug thereof, administered either alone or in combination, optionally in a pharmaceutically acceptable carrier. See also U.S. patent Publication Nos. 2004/0006002 and 2004/0006007 as well as WO 03/026589 and WO 03/026675. Idenix Pharmaceuticals, Ltd. also discloses in U.S. patent Publication No. 2004/0077587 pharmaceutically acceptable branched nucleoside prodrugs, and their use in the treatment of HCV and flaviviruses and pestiviruses in prodrugs. See also PCT Publication Nos. WO 04/002422, WO 04/002999, WO 04/003000; WO 04/024095 and WO 05/009418.

International Patent Publication WO 03/072757 to Biota Inc. discloses various phosphate derivatives of nucleosides, including 1′, 2′, 3′ or 4′-branched β-D or β-L nucleosides, for the treatment of hepatitis C infection.

Emory University and the University of Georgia Research Foundation, Inc. (UGARF) discloses the use of 2′-fluoronucleosides for the treatment of HCV in U.S. Pat. No. 6,348,587. See also U.S. patent Publication No. 2002/0198171 and International Patent Publication WO 99/43691.

BioChem Pharma Inc. (now Shire Biochem, Inc.) discloses the use of various 1,3-dioxolane nucleosides for the treatment of a Flaviviridae infection in U.S. Pat. No. 6,566,365. See also U.S. Pat. Nos. 6,340,690 and 6,605,614; U.S. patent Publication Nos. 2002/0099072 and 2003/0225037, as well as International Publication No. WO 01/32153 and WO 00/50424.

BioChem Pharma Inc. also discloses various other 2′-halo, 2′-hydroxy and 2′-alkoxy nucleosides for the treatment of a Flaviviridae infection in U.S. patent Publication No. 2002/0019363 as well as International Publication No. WO 01/60315 (PCT/CA01/00197; filed Feb. 19, 2001).

ICN Pharmaceuticals, Inc. discloses various nucleoside analogs that are useful in modulating immune response in U.S. Pat. Nos. 6,495,677 and 6,573,248. See also WO 98/16184, WO 01/68663, and WO 02/03997.

U.S. Pat. No. 6,660,721; U.S. patent Publication Nos. 2003/083307 A1, 2003/008841 A1, and 2004/0110718; as well as International Patent Publication Nos. WO 02/18404; WO 02/100415, WO 02/094289 and WO 04/043159; filed by F. Hoffmann-La Roche A G, discloses various nucleoside analogs for the treatment of HCV RNA replication.

Pharmasset Ltd. discloses various nucleosides and antimetabolites for the treatment of a variety of viruses, including Flaviviridae, and in particular HCV, in U.S. patent Publication Nos. 2003/0087873, 2004/0067877, 2004/0082574, 2004/0067877, 2004/002479, 2003/0225029, and 2002/00555483, as well as International Patent Publication Nos. WO 02/32920, WO 01/79246, WO 02/48165, WO 03/068162, WO 03/068164 and WO 2004/013298.

Merck & Co., Inc. and Isis Pharmaceuticals disclose in U.S. Pat. No. 6,777,395, issued Aug. 17, 2004; U.S. patent Publication No. 2004/0072788, 2004/0067901, and 2004/0110717; as well as the corresponding International Patent Publication Nos. WO 02/057425 (PCT/US02/01531; filed Jan. 18, 2002) and WO 02/057287 (PCT/US02/03086; filed Jan. 18, 2002) various nucleosides, and in particular several pyrrolopyrimidine nucleosides, for the treatment of viruses whose replication is dependent upon RNA-dependent RNA polymerase, including Flaviviridae, and in particular HCV. See also WO 2004/000858, WO 2004/003138, WO 2004/007512, and WO 2004/009020.

U.S. patent Publication No. 2003/028013 A1 as well as International Patent Publication Nos. WO 03/051899, WO 03/061576, WO 03/062255 WO 03/062256, WO 03/062257, and WO 03/061385, filed by Ribapharm, also are directed to the use of certain nucleoside analogs to treat hepatitis C virus.

Genelabs Technologies disclose in U.S. patent Publication No. 2004/0063658 as well as International Patent Publication Nos. WO 03/093290 and WO 04/028481 various base modified derivatives of nucleosides, including 1′, 2′, 3′ or 4′-branched β-D or β-L nucleosides, for the treatment of hepatitis C infection.

Anti-viral purines that have acyclic substituents are known and have been used to treat various viral infections. Examples of this class of compounds are acyclovir, ganciclovir, famciclovir, penciclovir, adefovir and adefovir dipivoxil, all of which are useful in the treatment of human syncytial virus (HSV), cytomegalo virus (CMV), and varicella-zoster virus (see EP 0 72027 to the Wellcome Foundation Ltd., UK, for treatment of equine rhinopneumonitis virus; JP 06227982 to Ajinomoto KK, for treatment of varicella-zoster virus and cytomegalovirus; S. Vittori et al., Deaza- and Deoxyadenosine Derivatives: Synthesis and Inhibition of Animal Viruses as Human Infection Models, in Nucleosides, Nucleotides & Nucleic Acids (2003) 22(5-8): 877-881, for treatment of bovine herpes virus 1 (BHV-1) and sheep Maedi-Visna Virus (MVV); R. Wang et al., Synthesis and biological activity of 2-aminopurine methylenecyclopropane analogues of nucleosides, in Nucleosides, Nucleotides & Nucleic Acids (2003) 22(2): 135-144, for treatment of HSV-1 and VZV; U.S. Pat. No. 6,444,656 to BioChem Pharma, Inc., Canada, for treatment of HIV and/or HBV infections; and WO 02/057288 to LG Chem Investment Ltd. for acyclic nucleoside phosphonate compounds for use as anti-HBV agents).

In view of the severity of diseases associated with pestiviruses, flaviviruses, and hepatitis C virus, and their pervasiveness in animals and humans, it is an object of the present invention to provide a compound, method and composition for the treatment of a host infected with any member of the family Flaviviridae, including hepatitis C virus.

Thus, it is an object of the present invention to provide a compound, method and pharmaceutically-acceptable composition for the prophylaxis and treatment of a host, and particularly a human, infected with any member of the family Flaviviridae.

### SUMMARY OF THE INVENTION

Methods and compositions for the treatment of pestivirus, flavivirus and hepatitis C virus infections are described that include administering an effective amount of a compound of Formulae (i), (ii), (iii), (iv), (v) or (vi): 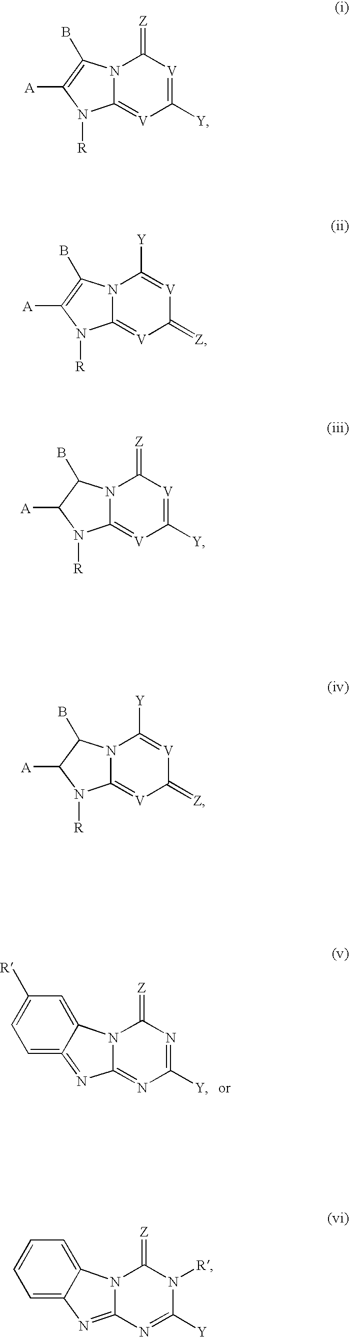
or a pharmaceutically acceptable salt or prodrug thereof, to a host in need thereof, wherein:

A, B and Y, each independently, is H; halogen; OR′, S(O)ₙ; S(O)ₙR′; S(O)ₙR′R″; NR′R″; NR; CN; CF₃; CR′R″; C(═W)OR′; C(═W)SR′; C(═W)NR′R′; C₁₋₄ alkylamino; di(C₁₋₄ alkyl)amino; C₃₋₆ cycloalkylamino; NO₂; N₃; C₁₋₁₀ cyclic or acyclic, branched or unbranched alkyl, alkenyl, alkynyl; aryl; aralkyl; heterocycle; or A and B taken together with the carbon atoms to which they are attached may form a 4-7 membered carbocyclic or heterocyclic ring;

Z is O, S, NR′, or CR′R″;

each V is independently N or CR′;

each R′ and R″ independently is H; C₁₋₁₀ cyclic or acyclic, branched or unbranched alkyl, alkenyl, alkynyl; halo; O-alkyl; NH₂; NHMe; N(Me)₂; CN; acyl; aryl; heteroaryl; heterocycle; carbocycle; amino acid residue; or together with the atoms to which they are attached may form a 3-7 membered carbocyclic or heterocyclic ring;

W is O, S, or NR′;

R is H; C₁₋₁₀ cyclic or acyclic, branched or unbranched alkyl, alkenyl, alkynyl, acyl, aryl, or aralkyl, any of which optionally may have one or more heteroatoms and any of which may be taken alone or in combination with one another; 3-7 membered carbocycle or heterocycle; or a functional group that dissociates to provide the base where R is H;

n is 0, 1 or 2;

wherein any branched or unbranched, cyclic or acyclic alkyl, alkenyl, or alkynyl may optionally comprise at least one heteroatom selected from the group consisting of O, S, N and P;

wherein any branched or unbranched, cyclic or acyclic alkyl, alkenyl, alkynyl; aryl; acyl; or aralkyl may optionally be substituted with one or more of OR′, SR′, NR′R″, halogen, NO₂, CN, N₃, CF₃, C(═W)OR′, C(═W)NR′R″, C(═W)SR′, alkyl, alkenyl, alkynyl, aryl, aralkyl, acyl, heterocycle or heteroatom selected from the group consisting of O, S, N and P; and

all tautomeric, enantiomeric and stereoisomeric forms thereof,

with the caveat that in Formula (i), when A and B are both H, each V is N, Z is O, and Y is —NH₂, then the compound is not β-D-2′-deoxy-5-aza-7-deazaguanosine, β-D-5-aza-7-deazaguanosine, β-D-5′-methyl-5-aza-7-deazaguanosine, or 2-amino-8-(methylpivalate)imidazo[1,2-a]-s-triazin-4-one.

Certain embodiments of the compounds of the present invention include a compound of Formula (i), (ii), (iii), (iv), (v) or (vi) wherein A, B, R′, R″, V, Y and Z are as defined above, and

R is selected from the group consisting of:

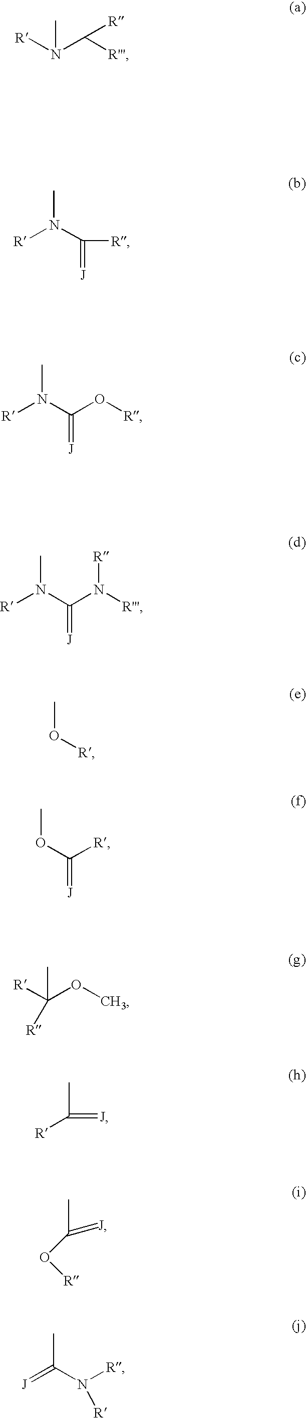

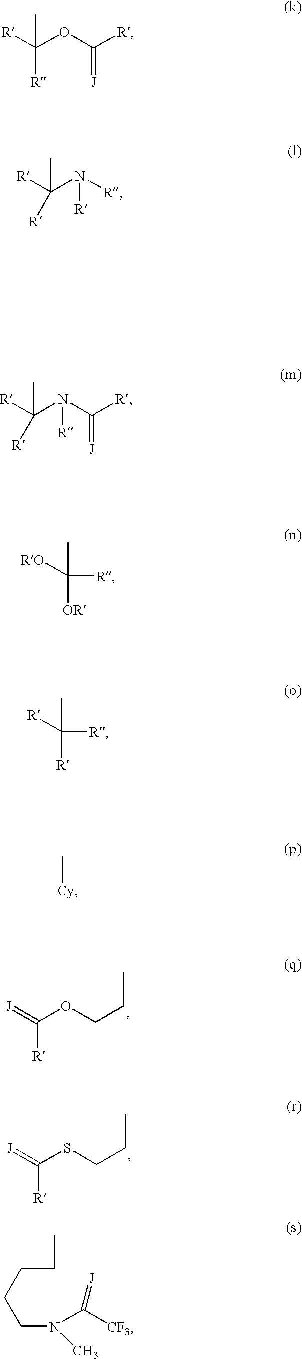

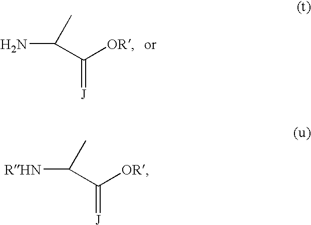

wherein J is O, S or N—R′;

Cy is any optionally substituted carbocycle, heterocycle or heteroaryl; and

R′″ is H, OH, SH, halo, optionally substituted C₁₋₄ alkyl, optionally substituted C₂₋₄ alkenyl or C₂₋₄ alkynyl, N₃, CN, CH₂CN, CH₂N₃, CH₂NH₂, CH₂NHCH₃, CH₂N(CH₃)₂, CH₂OH, halogenated alkyl, alkoxy, CF₃, C(A′)₃, 2-Br-ethyl, CH₂F, CH₂Cl, CH₂CF₃, CF₂CF₃, CH₂(A′), C(A)₂(A′)₃, haloalkenyl, Br-vinyl, haloalkynyl; —(CH₂)ₘC(O)OR⁴, —O(acyl), —O(lower acyl), —O(alkyl), —O(lower alkyl), —O(alkenyl), CF₃, halogen, —NO₂, —NH₂, —(CH₂)ₘNHR⁴, —(CH₂)ₘN(R⁴)₂, —(CH₂)ₘC(O)NHR⁴, —(CH₂)ₘC(O)N(R⁴)₂, or C₃₋₇ cycloalkylamino, and where the optional substitutions on alkyl, alkenyl and/or alkynyl may be one or more halogen, hydroxy, amino, alkoxy, or alkylthio groups or atoms taken in any combination.

In specific subembodiments of the invention, R′ and R″ can be selected from the group consisting of a structure depicted by any of the following formulae (I)-(VIII): 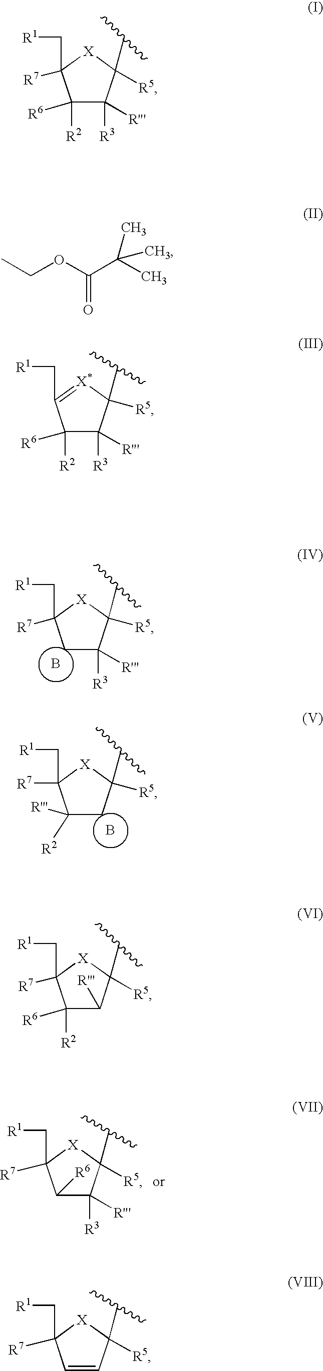
wherein:

R¹ is OH, phosphate or phosphonate (including mono-, di-, or triphosphate or a stabilized phosphate prodrug); acyl (including lower acyl); alkyl (including lower alkyl); sulfonate ester including alkyl or arylalkyl sulfonyl including methanesulfonyl and benzyl, wherein the phenyl group is optionally substituted with one or more substituents as described in the definition of an aryl given herein; optionally substituted arylsulfonyl; a lipid, including a phospholipid; an amino acid; a carbohydrate; a peptide; or cholesterol, of which any of the foregoing may be O-linked at the 5′-position on the ring structure; or other pharmaceutically acceptable leaving group that, in vivo, provides a compound wherein R′ is independently OH or O-phosphate;

each R² and R³ independently is H, OH, halo, NO₂, NH₂, N₃, CH₂N₃, CH₂NH₂, CN, CH₂CN, CH₂N₃, CH₂NHCH₃, CH₂N(CH₃)₂, CH₂OH, halogenated alkyl, alkoxy, CF₃, C(A′)₃, 2-Br-ethyl, CH₂F, CH₂Cl, CH₂CF₃, CF₂CF₃, CH₂(A′), C(A′)₂(A′)₃, SCN, OCN, NCO, haloalkenyl, Br-vinyl, haloalkynyl; —(CH₂)ₘC(O)OR⁴, —(CH₂)ₘC(O)SR⁴; —O(alkenyl), CF₃, halogen, —(CH₂)ₘNHR⁴, —(CH₂)ₘN(R⁴)₂, —(CH₂)ₘC(O)NHR⁴, —(CH₂)ₘC(O)N(R⁴)₂, —C(O)OR⁴, —O(R⁴), an optionally substituted carbocycle (typically a 3-7 membered carbocyclic ring such as, for example, a C₃₋₇ cycloalkylamino), an optionally substituted heterocycle (typically a 3-7 membered heterocyclic ring having one or more O, S and/or N), an optionally substituted heteroaryl (typically a heteroaromatic ring having one or more O, S and/or N atoms), a C₃₋₇ cycloalkylamino, and where CF₃, mercapto, optionally substituted C₁₋₄ alkyl, C₁₋₁₂ alkoxy, C₂₋₄alkenyl, or C₂₋₄ alkynyl, C₂₋₆ alkenyloxy, C₁₋₄ alkylthio, C₁₋₈ alkylcarbonyloxy, aryloxycarbonyl, C₁₋₄ alkylamino, di(C₁₋₄ alkyl)amino, Br-vinyl, —C(O)O(alkyl), O-phosphate or O-phosphonate (including mono-, di-, or triphosphate or a stabilized phosphate prodrug); O-acyl (including lower acyl); O-alkyl (including lower alkyl); O-sulfonate ester including O-alkyl or O-arylalkyl sulfonyl including O-methanesulfonyl and O-benzyl, wherein the phenyl group is optionally substituted with one or more substituents as described in the definition of an aryl given herein; —OC(O)O-aryl; —OC(O)O-aralkyl; —S(acyl); —S(alkyl); —S(alkenyl); optionally substituted O-arylsulfonyl; an O-linked lipid, including an O-phospholipid; an O-linked amino acid; an O-linked carbohydrate; an O-linked peptide; O-linked cholesterol; or other O-linked pharmaceutically acceptable leaving group that in vivo provides a compound wherein R¹ is independently H or phosphate;

R⁴ is H, alkyl, alkenyl, alkynyl, acyl, aryl or aralkyl;

each R⁵ and R⁶, independently, is H, —OH, —SH, —NH₂, —CF₃, Cl, F, Br, I, optionally substituted alkyl, optionally substituted alkenyl or alkynyl, —CH₂OH, alkoxy, CH₂F, CH₂N₃, CH₂CN, —(CH₂)ₘC(O)OR⁴, —(CH₂)ₘC(O)NHR⁴, —(CH₂)ₘC(O)N(R⁴)₂, —NH(alkyl), —N(alkyl)₂, —NH(acyl), —N(acyl)₂, or C₃₋₇ cycloalkylamino;

R⁷ is H, —OR¹, —OH, —NO₂, —CF₃, —NH₂, Cl, F, Br, I, N₃, CN, optionally substituted alkyl, optionally substituted alkenyl or alkynyl, Br-vinyl, —CH₂OH, —O(R⁴), alkoxy, —(CH₂)ₘC(O)O(R⁴), —OC(O)O-aryl, —OC(O)O-aralkyl, —SR⁴, —(CH₂)ₘNHR⁴, —(CH₂)ₘN(R⁴)₂, or C₃₋₇ cycloalkylamino;

X is O, S, SO₂, CH₂, CHOH, CH-halogen, C-(halogen)₂;

X* is CH, C—OH, or C-halogen;

m is 0, 1 or 2;

R′″ is as defined above; or R′″ and R³, together with the carbon atom to which they are attached, form an optionally substituted 3- to 7-membered saturated or unsaturated ring that optionally may have one or more heteroatoms selected from the group consisting of O, S, N or P;

except that R⁵ is OH, NH₂, or SH only when X or X* is C in Formulae I and III-VIII; [symbol] is an optionally substituted carbocycle typically a 3-7 membered carbocyclic ring, or an optionally substituted heterocycle, typically a 3-7 membered heterocyclic ring having one or more O, S and/or N, that forms a spiro-nucleoside;

A′ is H, OH, C₁₋₄ alkyl, halo, azido, cyano, C₂₋₆ alkenyl, C₂₋₆ alkynyl, Br-vinyl, 2-Br-ethyl, —C(O)O(alkyl), —C(O)O(lower alkyl), —O(acyl), —O(lower acyl), —O(alkyl), —O(lower alkyl), —O(alkenyl), CF₃, NO₂, NH₂, —NH(lower alkyl), —NH(acyl), —N(lower alkyl)₂, or —N(acyl)₂; and

all tautomeric, enantiomeric and stereoisomeric forms thereof.

The active compounds of the present invention can be administered in combination, alternation or sequential steps with another anti-viral, and typically an anti-HCV, agent. In combination therapy, effective dosages of two or more agents are administered together, whereas in alternation or sequential-step therapy, an effective dosage of each agent is administered serially or sequentially. The dosages given will depend on absorption, inactivation and excretion rates of the drug as well as other factors known to those of skill in the art. It is to be noted that dosage values will also vary with the severity of the condition to be alleviated. It is to be further understood that for any particular subject, specific dosage regimens and schedules should be adjusted over time according to the individual need and the professional judgment of the person administering or supervising the administration of the compositions. In some embodiments, an anti-HCV (anti-pestivirus or anti-flavivirus) compound that exhibits an EC₅₀ of 10-15 μM, or typically less than 1-5 μM, is desirable.

HCV is a member of the family, Flaviviridae; however, HCV now has been placed in a new monotypic genus, hepacivirus. Therefore, in one embodiment of the present invention, the flavivirus or pestivirus is not HCV, while in another embodiment, the genus, hepacivirus, is embraced.

In specific embodiments, the present invention provides the following:
a) a compound comprising any one of Formulae (i), (ii), (iii), (iv), (v) or (vi), or its pharmaceutically acceptable salt or prodrug thereof;
b) a compound comprising any one of Formulae (i), (ii), (iii), (iv), (v) or (vi), or a pharmaceutically acceptable salt or prodrug thereof, substantially in the absence of enantiomers of the described nucleoside, or substantially isolated from other chemical entities, or substantially in the form of a single enantiomer;
c) a pharmaceutical composition or formulation comprising any one of Formulae (i), (ii), (iii), (iv), (v) or (vi), or a pharmaceutically acceptable salt or prodrug thereof, together with a pharmaceutically acceptable carrier, excipient or diluent;
d) a pharmaceutical composition or formulation comprising any one of Formulae (i), (ii), (iii), (iv), (v) or (vi), or a pharmaceutically acceptable salt or prodrug thereof, with one or more other effective antiviral agents, optionally with a pharmaceutically acceptable carrier or diluent;
e) a pharmaceutical composition for the treatment or prophylaxis of a pestivirus, flavivirus or HCV infection in a host, especially a host diagnosed as having or being at risk for such infection, comprising any one of Formulae (i), (ii), (iii), (iv), (v) or (vi), or a pharmaceutically acceptable salt or prodrug thereof, together with a pharmaceutically acceptable carrier or diluent;
f) a method for the treatment of a pestivirus, flavivirus or HCV infection in a host comprising administering a compound of any one of Formulae (i), (ii), (iii), (iv), (v) or (vi), or a pharmaceutically acceptable salt or prodrug thereof, optionally with a pharmaceutically acceptable carrier, excipient or diluent;
g) a method for the treatment of a pestivirus, flavivirus or HCV infection in a host comprising administering a compound of any one of Formulae (i), (ii), (iii), (iv), (v) or (vi), or a pharmaceutically acceptable salt or prodrug thereof, with one or more other effective antiviral agents, optionally with a pharmaceutically acceptable carrier, excipient or diluent;
h) use of a compound comprising any one of Formulae (i), (ii), (iii), (iv), (v) or or (vi), or a pharmaceutically acceptable salt or prodrug thereof, optionally with a pharmaceutically acceptable carrier or diluent, for the treatment of a pestivirus, flavivirus or HCV infection in a host, or its use in the manufacture of a medicament for treatment of the infection;
i) use of a compound comprising any one of Formulae (i), (ii), (iii), (iv), (v) or (vi), or a pharmaceutically acceptable salt or prodrug thereof, with one or more other effective antiviral agents, optionally with a pharmaceutically acceptable carrier or diluent, for the treatment of a pestivirus, flavivirus and/or HCV infection in a host, or its use in the manufacture of a medicament for treatment of the infection;
j) a process for the preparation of a compound comprising any one of Formulae (i), (ii), (iii), (iv), (v), or (vi), or a pharmaceutically acceptable salt or prodrug thereof, as provided in more detail below; and
k) a process for the preparation of a compound comprising any one of Formulae (i), (ii), (iii), (iv), (v), or (vi), or a pharmaceutically acceptable salt or prodrug thereof, substantially in the absence of enantiomers of the described compound or substantially isolated from other chemical entities.

### DETAILED DESCRIPTION OF THE INVENTION

The present invention provides a compound, method and composition for the treatment of a pestivirus, flavivirus and/or hepatitis C in humans or other host animals that includes a compound any one of Formulae (i), (ii), (iii), (iv), (v) or (vi), given below. In one typical embodiment of these compounds, methods an compositions, the R substituent is H; C₁₋₁₀ cyclic or acyclic, branched or unbranched alkyl, alkenyl, alkynyl; acyl; aryl; aralkyl; 3-7 membered carbocycle or heterocycle; a functional group that dissociates to provide the compound of Formulae (i), (ii), (iii), (iv), (v) or (vi) where R is H; a structure of formulae (I)-(VIII) also given below, or a pharmaceutically acceptable salt or prodrug thereof, optionally in a pharmaceutically acceptable carrier. A compound of this invention either possesses antiviral activity, or is metabolized to a compound that exhibits such activity. Methods are provided that include administering an effective anti-pestivirus, anti-flavivirus or anti-HCV treatment amount of a compound of the present invention to a host.

### DEFINITIONS

The term “alkyl” as used herein, unless otherwise specified, includes, but is not limited to, a saturated straight, branched, or cyclic, primary, secondary, or tertiary hydrocarbon of typically C₁ to C₁₀, and specifically includes methyl, trifluoromethyl, ethyl, propyl, isopropyl, cyclopropyl, butyl, isobutyl, t-butyl, pentyl, cyclopentyl, isopentyl, neopentyl, hexyl, isohexyl, cyclohexyl, cyclohexylmethyl, 3-methylpentyl, 2,2-dimethybutyl, and 2,3-dimethylbutyl. The term includes both substituted and unsubstituted alkyl groups. Moieties with which the alkyl group can be substituted with one or more substituents include but are not limited to halo, including Cl, F, Br and I so as to form, for eg., CF₃, 2-Br-ethyl, CH₂F, CH₂Cl, CH₂CF₃, or CF₂CF₃; hydroxyl, for eg. CH₂OH; amino, for eg., CH₂NH₂, CH₂NHCH₃, or CH₂N(CH₃)₂; carboxylate; carboxamido; alkylamino; arylamino; alkoxy; aryloxy; nitro; azido, for eg., CH₂N₃; cyano, for eg., CH₂CN; thio; sulfonic acid; sulfate; phosphonic acid; phosphate; and phosphonate, either unprotected or protected as necessary, known to those skilled in the art, for eg., as taught in Greene et al., Protective Groups in Organic Synthesis, John Wiley and Sons, Second Edition (1991), incorporated herein by reference.

In the specification, when a range is specified, the term independently includes every member of the range. For example, when the specification refers to “C₁₋₁₀ alkyl”, this includes independently C₁, C₂, C₃, C₄, C₅, C₆, C₇, C₈, C₉ and C₁₀ alkyl.

The term “lower alkyl” as used herein, and unless otherwise specified, includes a C₁ to C₆ saturated straight, branched, or if appropriate, cyclic as in cyclopropyl, for eg., alkyl group, including both substituted and unsubstituted forms. Unless otherwise specifically stated in this application, when alkyl is a suitable moiety, lower alkyl is typical. Similarly, when alkyl or lower alkyl is a suitable moiety, unsubstituted alkyl or lower alkyl is typical.

The terms “alkylamino” and “arylamino” refer to an amino group that has one or two alkyl or aryl substituents, respectively.

The term “protected” as used herein and, unless otherwise defined, includes a group that is added to an oxygen, nitrogen or phosphorus atom to prevent its further reaction or for other purposes. Numerous oxygen and nitrogen protecting groups are known to those skilled in the art of organic synthesis.

The term “aryl” as used herein and, unless otherwise specified, includes phenyl, biphenyl or naphthyl, and typically is phenyl. The term includes both substituted and unsubstituted moieties. The aryl group can be substituted with one or more moieties including but not limited to alkyl, hydroxyl, amino, alkylamino, arylamino, alkoxy, aryloxy, nitro, cyano, thio, alkylthio, carboxamido, carboxylate, sulfonic acid, sulfate, phosphonic acid, phosphate, or phosphonate, either unprotected or protected as necessary, as known to those skilled in the art, for eg., as taught in Greene et al., Protective Groups in Organic Synthesis, John Wiley and Sons, Second Edition (1991), incorporated herein by reference.

The term “3 to 7-membered carbocyclic or heterocyclic ring” as used herein is meant to include monocyclic and polycyclic ring structures that are carbocycles, heterocycles or one or more carbocycles in combination with one or more heterocycles, any of which optionally may be substituted.

The terms “alkaryl” and “akylaryl” refer to an alkyl group with an aryl sustituent. The terms “aralkyl” and “arylalkyl” refer to an aryl group with an alkyl substituent.

The term “halo” as used herein includes bromo, chloro, iodo and fluoro.

The term “acyl” includes a carboxylic acid ester in which the non-carbonyl moiety of the ester group is selected from straight, branched, or cyclic alkyl or lower alkyl; alkoxyalkyl including methoxymethyl; aralkyl including benzyl; aryloxyalkyl such as phenoxymethyl; aryl including phenyl optionally substituted with halogen, C₁-C₆ alkyl or C₁-C₆ alkoxy; sulfonate esters such as alkyl or aralkyl sulphonyl including methanesulfonyl; the mono-, di- or triphosphate ester; trityl or monomethoxytrityl; substituted benzyl; trialkylsilyl as, for eg., dimethyl-t-butylsilyl or diphenylmethylsilyl. Aryl groups in the esters optimally comprise a phenyl group. The term “lower acyl” includes an acyl group in which the non-carbonyl moiety is lower alkyl.

The term “nucleoside analog” as used herein includes a compound having an optionally substituted, naturally occurring purine base such as adenine or guanine, or an optionally substituted, non-naturally occurring base such as, for example, a 5-aza-7-deazapurine base, bonded at position 9 to an acyclic, carbocyclic or heterocyclic moiety that is not a furanosyl, ribofuranosyl or arabinofuransyl ring.

The phrase “functional group that dissociates to provide the base where R is H” or “functional group that dissociates to provide the compound where R is H” as used herein refers to any “R” substituent whose bond to the 5-aza-7-deazapurine base or base derivative can be broken to release the free base or base derivative. Non-limiting examples of such “R” substituent groups include acyl, carboxylic acid, carboxylic acid ester, carboxamido, and thio-ester groups that can undergo hydrolysis to provide the free base or base derivative.

The following abbreviations for certain reagents used in the working examples and their definitions are: DCM is dichloromethane; DCE is dichloroethane; DMF is dimethylformamide; TFA is trifluoroacetyl; TMSCl is trimethylsilyl chloride; TsCl is tosyl chloride; and TFA is trifluoroacetyl.

As used herein, the terms “substantially free of” and “substantially in the absence of” with respect to a nucleoside composition refer to a nucleoside composition that includes at least 85 or 90% by weight, typically at least 95% or 98% by weight, and even more typically at least 99% or 100% by weight, of the designated enantiomer of that nucleoside. In a certain embodiment, the compounds listed in the methods and compounds of this invention are substantially free of enantiomers other than for the one designated.

Similarly, the term “isolated” with respect to a compound composition such as a nucleoside composition, refers to a composition that includes at least 85% or 90% by weight, typically 95% or 98% by weight, and even more typically 99% or 100% by weight, of the compound, such as a nucleoside.

The term “host”, as used herein, refers to a unicellular or multicellular organism in which the virus can replicate, including cell lines and animals, and typically a human. Alternatively, the host can be carrying a part of the flavivirus or pestivirus genome, whose replication or function can be altered by the compounds of the present invention. The term host specifically refers to infected cells, cells transfected with all or part of the flavivirus or pestivirus genome and animals, in particular, primates (including chimpanzees) and humans. In most animal applications of the present invention, the host is a human patient. Veterinary applications, in certain indications, however, are clearly anticipated by the present invention such as in chimpanzees.

The term “pharmaceutically acceptable salt or prodrug” is used throughout the specification to describe any pharmaceutically acceptable form (ester, phosphate ester, salt of an ester or a related group) of a nucleoside compound, which, upon administration to a patient, provides the nucleoside compound. Pharmaceutically acceptable salts include those derived from pharmaceutically acceptable inorganic or organic bases and acids. Suitable salts include those derived from alkali metals such as potassium and sodium, alkaline earth metals such as calcium and magnesium, among numerous other acids well known in the pharmaceutical art. Pharmaceutically acceptable prodrugs refer to a compound that is metabolized, for example, hydrolyzed or oxidized, in the host to form the compound of the present invention. Typical examples of prodrugs include compounds that have biologically labile protecting groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, dephosphorylated to produce the active compound. The compounds of this invention possess antiviral activity against flavivirus, pestivirus or HCV, or are metabolized to a compound that exhibits such activity.

Active Compounds, Physiologically Acceptable Salts and Prodrugs Thereof

Methods and compositions for the treatment of pestivirus, flavivirus and hepatitis C virus infection are described that include administering an effective amount of a compound comprising any one of Formulae (i), (ii), (iii), (iv), (v) or (vi): 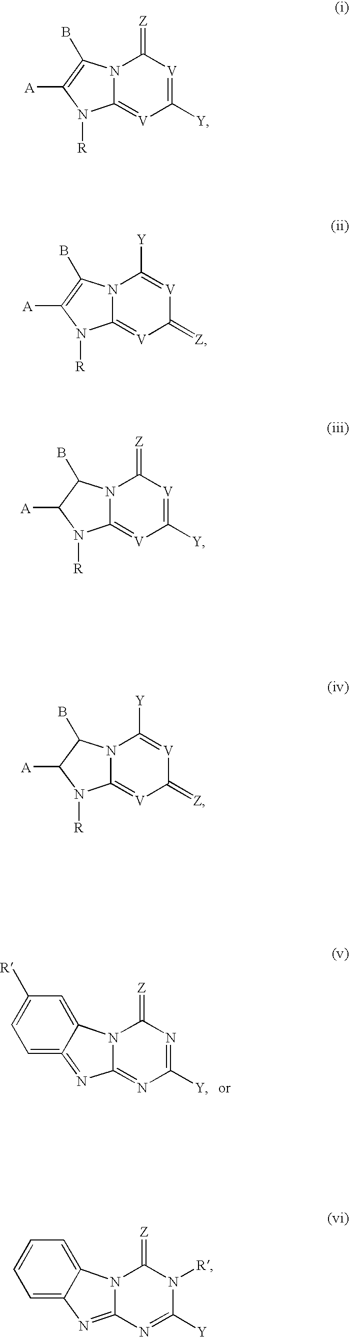
or a pharmaceutically acceptable salt or prodrug thereof, to a host in need thereof, wherein

A, B and Y, each independently, is H; halogen; OR′, S(O)ₙ; S(O)ₙR′; S(O)ₙR′R″; NR′R″; NR; CN; CF₃; CR′R″; C(═W)OR′; C(═W)SR′; C(═W)NR′R′; C₁₋₄ alkylamino; di(C₁₋₄ alkyl)amino; C₃₋₆ cycloalkylamino; NO₂; N₃; C₁₋₁₀ cyclic or acyclic, branched or unbranched alkyl, alkenyl, alkynyl; aryl; aralkyl; heterocycle; or A and B taken together with the carbon atoms to which they are attached may form a 4-7 membered carbocyclic or heterocyclic ring;

Z is O, S, NR′, or CR′R″;

each V is independently N or CR′;

each R′ and R″ independently is H; C₁₋₁₀ cyclic or acyclic, branched or unbranched alkyl, alkenyl, alkynyl; halo; O-alkyl; NH₂; NHMe; N(Me)₂; CN; acyl; aryl; heteroaryl; heterocycle; carbocycle; amino acid residue; or together with the atoms to which they are attached may form a 3-7 membered carbocyclic or heterocyclic ring;

each W is independently O, S, or NR′;

each R is independently H; C₁₋₁₀ cyclic or acyclic, branched or unbranched alkyl, alkenyl, alkynyl, acyl, aryl, or aralkyl, any of which optionally may have one or more heteroatoms and any of which may be taken alone or in combination with one another; 3-7 membered carbocycle or heterocycle; a functional group that dissociates to provide the compound where R is H; or a structure depicted by any of the formulae (I)-(VIII) provided below;

n is 0, 1 or 2;

wherein any branched or unbranched, cyclic or acyclic alkyl, alkenyl, or alkynyl may optionally comprise at least one heteroatom selected from the group consisting of O, S, N and P;

wherein any branched or unbranched, cyclic or acyclic alkyl, alkenyl, alkynyl; aryl; acyl; or aralkyl may optionally be substituted with one or more of OR′, SR′, NR′R″, halogen, NO₂, CN, N₃, CF₃, C(═W)OR′, C(═W)NR′R″, C(═W)SR′, alkyl, alkenyl, alkynyl, aryl, aralkyl, acyl, heterocycle or heteroatom selected from the group consisting of O, S, N and P; and

all tautomeric, enantiomeric and stereoisomeric forms thereof,

with the caveat that in Formula (i), when A and B are both H, each V is N, Z is O, and Y is —NH₂, then Formula (i) is not β-D-2′-deoxy-5-aza-7-deazaguanosine, β-D-5-aza-7-deazaguanosine, β-D-5′-methyl-5-aza-7-deazaguanosine, or 2-amino-8-(methyl-pivaloyl)imidazo[1,2-a]-s-triazin-4-one.

In a first principal embodiment, a compound of the Formula (i), (iii), or (v), or a pharmaceutically acceptable salt or prodrug thereof, is provided: 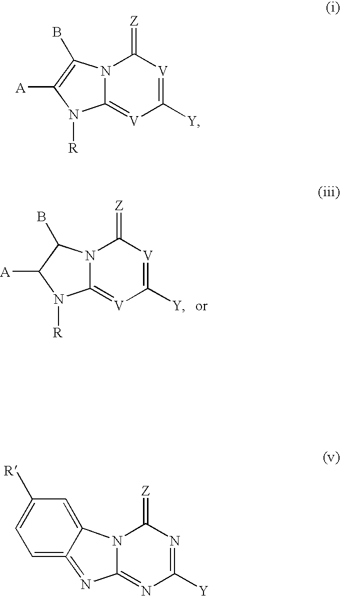
wherein:

A, B and Y, each independently, is H; halogen; OR′, S(O)ₙ; S(O)ₙR′; S(O)ₙR′R″; NR′R″; NR; CN; CF₃; CR′R″; C(═W)OR′; C(═W)SR′; C(═W)NR′R′; C₁₋₄ alkylamino; di(C₁₋₄ alkyl)amino; C₃₋₆ cycloalkylamino; NO₂; N₃; C₁₋₁₀ cyclic or acyclic, branched or unbranched alkyl, alkenyl, alkynyl; aryl; aralkyl; heterocycle; or A and B taken together with the carbon atoms to which they are attached may form a 4-7 membered carbocyclic or heterocyclic ring; but wherein at least one of A or B is not H;

Z is O, S, NR′, or CR′R″;

each V is independently N or CR′;

each R′ and R″ independently is H; C₁₋₁₀ cyclic or acyclic, branched or unbranched alkyl, alkenyl, alkynyl; halo; O-alkyl; NH₂; NHMe; N(Me)₂; CN; acyl; aryl; heteroaryl; heterocycle; carbocycle; amino acid residue; or together with the atoms to which they are attached may form a 3-7 membered carbocyclic or heterocyclic ring;

each W is independently O, S, or NR′;

each n is independently 0, 1 or 2;

wherein any branched or unbranched, cyclic or acyclic alkyl, alkenyl, or alkynyl may optionally comprise at least one heteroatom selected from the group consisting of O, S, N and P;

wherein any branched or unbranched, cyclic or acyclic alkyl, alkenyl, alkynyl; aryl; acyl; or aralkyl may optionally be substituted with one or more of OR′, SR′, NR′R″, halogen, NO₂, CN, N₃, CF₃, C(═W)OR′, C(═W)NR′R″, C(═W)SR′, alkyl, alkenyl, alkynyl, aryl, aralkyl, acyl, heterocycle or heteroatom selected from the group consisting of O, S, N and P; and

R is

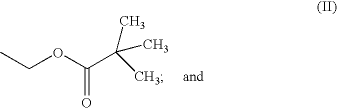

all tautomeric, enantiomeric and stereoisomeric forms thereof

In a second principal embodiment, a compound of the Formula (i), (iii), or (vi), or a pharmaceutically acceptable salt or prodrug thereof, is provided:

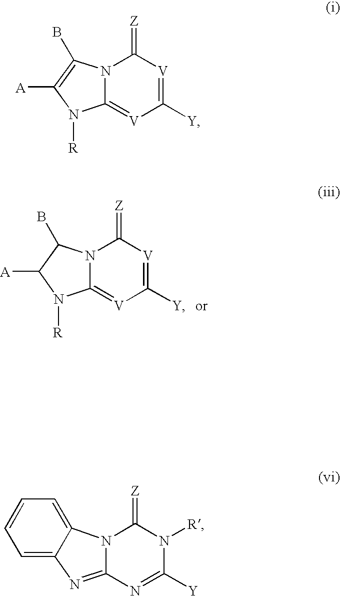

wherein

A, B and Y, each independently, is H; halogen; OR′, S(O)ₙ; S(O)ₙR′; S(O)ₙR′R″; NR′R″; NR; CN; CF₃; CR′R″; C(═W)OR′; C(═W)SR′; C(═W)NR′R′; C₁₋₄ alkylamino; di(C₁₋₄ alkyl)amino; C₃₋₆ cycloalkylamino; NO₂; N₃; C₁₋₁₀ cyclic or acyclic, branched or unbranched alkyl, alkenyl, alkynyl; aryl; aralkyl; heterocycle; or A and B taken together with the carbon atoms to which they are attached may form a 4-7 membered carbocyclic or heterocyclic ring; with the caveat that A and B may not both be H;

Z is O, S, NR′, or CR′R″;

each V is independently N or CR′;

each R′ and R″ independently is H; C₁₋₁₀ cyclic or acyclic, branched or unbranched alkyl, alkenyl, alkynyl; halo; O-alkyl; NH₂; NHMe; N(Me)₂; CN; acyl; aryl; heteroaryl; heterocycle; carbocycle; amino acid residue; or together with the atoms to which they are attached may form a 3-7 membered carbocyclic or heterocyclic ring;

W is O, S, or NR′;

each n is independently 0, 1 or 2;

wherein any branched or unbranched, cyclic or acyclic alkyl, alkenyl, or alkynyl may optionally comprise at least one heteroatom selected from the group consisting of O, S, N and P;

wherein any branched or unbranched, cyclic or acyclic alkyl, alkenyl, alkynyl; aryl; acyl; or aralkyl may optionally be substituted with one or more of OR′, SR′, NR′R″, halogen, NO₂, CN, N₃, CF₃, C(═W)OR′, C(═W)NR′R″, C(═W)SR′, alkyl, alkenyl, alkynyl, aryl, aralkyl, acyl, heterocycle or heteroatom selected from the group consisting of O, S, N and P; and

R is: 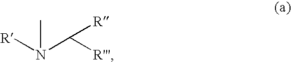
wherein

each R′ and R″ independently is H; C₁₋₁₀ cyclic or acyclic, branched or unbranched alkyl, alkenyl, alkynyl; halo; O-alkyl; NH₂; NHMe; N(Me)₂; CN; acyl; aryl; heteroaryl; heterocycle; carbocycle; amino acid residue; or together with the atoms to which they are attached may form a 3-7 membered carbocyclic or heterocyclic ring;

R′″ is H, OH, SH, halo, optionally substituted C₁₋₄ alkyl, optionally substituted C₂₋₄ alkenyl or C₂₋₄ alkynyl, N₃, CN, CH₂CN, CH₂N₃, CH₂NH₂, CH₂NHCH₃, CH₂N(CH₃)₂, CH₂OH, halogenated alkyl, alkoxy, CF₃, C(A′)₃, 2-Br-ethyl, CH₂F, CH₂Cl, CH₂CF₃, CF₂CF₃, CH₂(A′), C(A)₂(A′)₃, haloalkenyl, Br-vinyl, haloalkynyl; —(CH₂)ₘC(O)OR⁴, —O(acyl), —O(lower acyl), —O(alkyl), —O(lower alkyl), —O(alkenyl), CF₃, halogen, —NO₂, —NH₂, —(CH₂)ₘNHR⁴, —(CH₂)ₘN(R⁴)₂, —(CH₂)ₘC(O)NHR⁴, —(CH₂)ₘC(O)N(R⁴)₂, or C₃₋₇ cycloalkylamino, and where the optional substitutions on alkyl, alkenyl and/or alkynyl may be one or more halogen, hydroxy, amino, alkoxy, or alkylthio groups or atoms taken in any combination; and

all tautomeric, enantiomeric and stereoisomeric forms thereof.

In a third principal embodiment, a compound of the Formula (i), (iii), or (v), or a pharmaceutically acceptable salt or prodrug thereof, is provided: 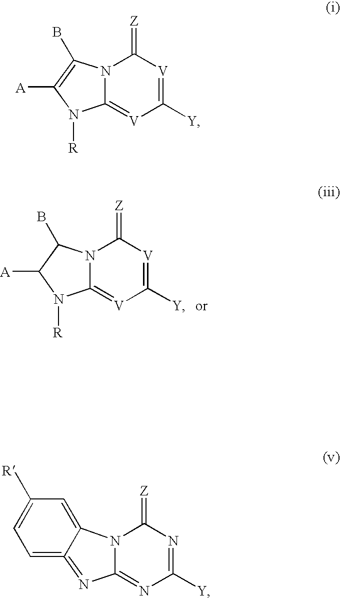
wherein:

A, B and Y, each independently, is H; halogen; OR′, S(O)ₙ; S(O)ₙR′; S(O)ₙR′R″; NR′R″; NR; CN; CF₃; CR′R″; C(═W)OR′; C(═W)SR′; C(═W)NR′R′; C₁₋₄ alkylamino; di(C₁₋₄ alkyl)amino; C₃₋₆ cycloalkylamino; NO₂; N₃; C₁₋₁₀ cyclic or acyclic, branched or unbranched alkyl, alkenyl, alkynyl; aryl; aralkyl; heterocycle; or A and B taken together with the carbon atoms to which they are attached may form a 4-7 membered carbocyclic or heterocyclic ring; with the caveat that A and B may not both be H;

Z is O, S, NR′, or CR′R″;

each V is independently N or CR′;

each R′ and R″ independently is H; C₁₋₁₀ cyclic or acyclic, branched or unbranched alkyl, alkenyl, alkynyl; halo; O-alkyl; NH₂; NHMe; N(Me)₂; CN; acyl; aryl; heteroaryl; heterocycle; carbocycle; amino acid residue; or together with the atoms to which they are attached may form a 3-7 membered carbocyclic or heterocyclic ring;

each W is independently O, S, or NR′;

each n is independently 0, 1 or 2;

wherein any branched or unbranched, cyclic or acyclic alkyl, alkenyl, or alkynyl may optionally comprise at least one heteroatom selected from the group consisting of O, S, N and P;

wherein any branched or unbranched, cyclic or acyclic alkyl, alkenyl, alkynyl; aryl; acyl; or aralkyl may optionally be substituted with one or more of OR′, SR′, NR′R″, halogen, NO₂, CN, N₃, CF₃, C(═W)OR′, C(═W)NR′R″, C(═W)SR′, alkyl, alkenyl, alkynyl, aryl, aralkyl, acyl, heterocycle or heteroatom selected from the group consisting of O, S, N and P; and

R is selected formula (I) or (III):

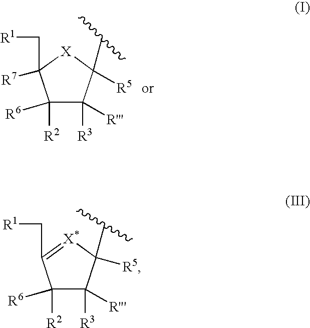

wherein:

R′ is OH, phosphate or phosphonate (including mono-, di-, or triphosphate or a stabilized phosphate prodrug); acyl (including lower acyl); alkyl (including lower alkyl); sulfonate ester including alkyl or arylalkyl sulfonyl including methanesulfonyl and benzyl, wherein the phenyl group is optionally substituted with one or more substituents as described in the definition of an aryl given herein; optionally substituted arylsulfonyl; a lipid, including a phospholipid; an amino acid; a carbohydrate; a peptide; or cholesterol, of which any of the foregoing may be O-linked at the 5′-position on the ring structure; or other pharmaceutically acceptable leaving group that, in vivo, provides a compound wherein R′ is independently OH or O-phosphate;

each R² and R³ independently is H, OH, halo, NO₂, NH₂, N₃, CH₂N₃, CH₂NH₂, CN, CH₂CN, CH₂N₃, CH₂NHCH₃, CH₂N(CH₃)₂, CH₂OH, halogenated alkyl, alkoxy, CF₃, C(A′)₃, 2-Br-ethyl, CH₂F, CH₂Cl, CH₂CF₃, CF₂CF₃, CH₂(A′), C(A′)₂(A′)₃, SCN OCN, NCO, haloalkenyl, Br-vinyl, haloalkynyl; —(CH₂)ₘC(O)OR⁴, —(CH₂)ₘC(O)SR⁴; —O(alkenyl), CF₃, halogen, —(CH₂)ₘNHR⁴, —(CH₂)ₘN(R⁴)₂, —(CH₂)ₘC(O)NHR⁴, —(CH₂)ₘC(O)N(R⁴)₂, —C(O)OR⁴, —O(R⁴), an optionally substituted carbocycle (typically a 3-7 membered carbocyclic ring such as, for example, a C₃₋₇ cycloalkylamino), an optionally substituted heterocycle (typically a 3-7 membered heterocyclic ring having one or more O, S and/or N), an optionally substituted heteroaryl (typically a heteroaromatic ring having one or more O, S and/or N atoms), a C₃₋₇ cycloalkylamino, and where CF₃, mercapto, optionally substituted C₁₋₄ alkyl, C₁₋₁₂ alkoxy, C₂₋₄alkenyl, or C₂₋₄ alkynyl, C₂₋₆ alkenyloxy, C₁₋₄ alkylthio, C₁₋₈ alkylcarbonyloxy, aryloxycarbonyl, C₁₋₄ alkylamino, di(C₁₋₄ alkyl)amino, Br-vinyl, —C(O)O(alkyl), O-phosphate or O-phosphonate (including mono-, di-, or triphosphate or a stabilized phosphate prodrug); O-acyl (including lower acyl); O-alkyl (including lower alkyl); O-sulfonate ester including O-alkyl or O-arylalkyl sulfonyl including O-methanesulfonyl and O-benzyl, wherein the phenyl group is optionally substituted with one or more substituents as described in the definition of an aryl given herein; —OC(O)O-aryl; —OC(O)O-aralkyl; —S(acyl); —S(alkyl); —S(alkenyl); optionally substituted O-arylsulfonyl; an O-linked lipid, including an O-phospholipid; an O-linked amino acid; an O-linked carbohydrate; an O-linked peptide; O-linked cholesterol; or other O-linked pharmaceutically acceptable leaving group that in vivo provides a compound wherein R′ is independently H or phosphate;

each R⁴ is independently H, alkyl, alkenyl, alkynyl, acyl, aryl or aralkyl;

each R⁵ and R⁶, independently, is H, —OH, —SH, —NH₂, —CF₃, Cl, F, Br, I, optionally substituted alkyl, optionally substituted alkenyl or alkynyl, —CH₂OH, alkoxy, CH₂F, CH₂N₃, CH₂CN, —(CH₂)ₘC(O)OR⁴, —(CH₂)ₘC(O)NHR⁴, (CH₂)ₘC(O)N(R⁴)₂, —NH(alkyl), —N(alkyl)₂, —NH(acyl), —N(acyl)₂, or C₃₋₇ cycloalkylamino;

R⁷ is H, —OR¹, —OH, —NO₂, —CF₃, —NH₂, Cl, F, Br, I, N₃, CN, optionally substituted alkyl, optionally substituted alkenyl or alkynyl, Br-vinyl, —CH₂OH, —O(R⁴), alkoxy, —(CH₂)ₘC(O)O(R⁴), —OC(O)O-aryl, —OC(O)O-aralkyl, —SR⁴, —(CH₂)ₘNHR⁴, —(CH₂)ₘN(R⁴)₂, or C₃₋₇ cycloalkylamino;

X is O, S, SO₂, CH₂, CHOH, CH-halogen, C-(halogen)₂;

X* is CH, C—OH, or C-halogen;

each m is independently 0, 1 or 2;

R′″ is H, OH, SH, halo, optionally substituted C₁₋₄ alkyl, optionally substituted C₂₋₄ alkenyl or C₂₋₄ alkynyl, N₃, CN, CH₂CN, CH₂N₃, CH₂NH₂, CH₂NHCH₃CH₂N(CH₃)₂, CH₂OH, halogenated alkyl, alkoxy, CF₃, C(A′)₃, 2-Br-ethyl, CH₂F, CH₂Cl, CH₂CF₃, CF₂CF₃, CH₂(A′), C(A′)₂(A′)₃, haloalkenyl, Br-vinyl, haloalkynyl; —(CH₂)ₘC(O)OR⁴, —O(acyl), —O(lower acyl), —O(alkyl), —O(lower alkyl), —O(alkenyl), CF₃, halogen, —NO₂, —NH₂, —(CH₂)ₘNHR⁴, —(CH₂)ₘN(R⁴)₂, —(CH₂)ₘC(O)NHR⁴, —(CH₂)ₘC(O)N(⁴)₂, or C₃₋₇ cycloalkylamino, and where the optional substitutions on alkyl, alkenyl and/or alkynyl may be one or more halogen, hydroxy, amino, alkoxy, or alkylthio groups or atoms taken in any combination; or

R′″ and R³, together with the carbon atom to which they are attached, form an optionally substituted 3- to 7-membered saturated or unsaturated ring that optionally may have one or more heteroatoms selected from the group consisting of O, S, N or P;

each A′ is independently H, OH, C₁₋₄ alkyl, halo, azido, cyano, C₂₋₆ alkenyl, C₂₋₆ alkynyl, Br-vinyl, 2-Br-ethyl, —C(O)O(alkyl), —C(O)O(lower alkyl), —O(acyl), —O(lower acyl), —O(alkyl), —O(lower alkyl), —O(alkenyl), CF₃, NO₂, NH₂, —NH(lower alkyl), —NH(acyl), —N(lower alkyl)₂, or —N(acyl)₂; and

all tautomeric, enantiomeric and stereoisomeric forms thereof.

In a fourth principal embodiment, a compound of Formula (i), (iii), or (vi), or a pharmaceutically acceptable salt or prodrug thereof, is provided: 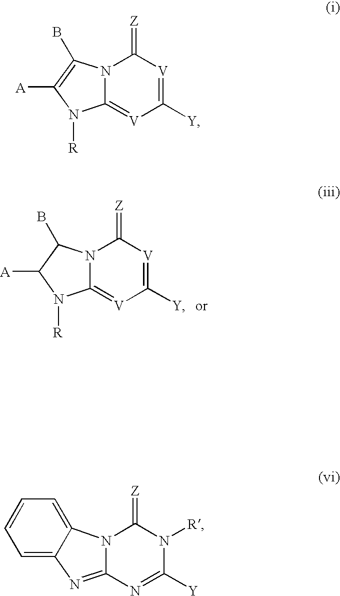
wherein:

A, B and Y, each independently, is H; halogen; OR′, S(O)ₙ; S(O)ₙR′; S(O)ₙR′R″; NR′R″; NR; CN; CF₃; CR′R″; C(═W)OR′; C(═W)SR′; C(═W)NR′R′; C₁₋₄ alkylamino; di(C₁₋₄ alkyl)amino; C₃₋₆ cycloalkylamino; NO₂; N₃; C₁₋₁₀ cyclic or acyclic, branched or unbranched alkyl, alkenyl, alkynyl; aryl; aralkyl; heterocycle; or A and B taken together with the carbon atoms to which they are attached may form a 4-7 membered carbocyclic or heterocyclic ring; with the caveat that A and B may not both be H;

Z is O, S, NR′, or CR′R″;

each V is independently N or CR′;

each R′ and R″ independently is H; C₁₋₁₀ cyclic or acyclic, branched or unbranched alkyl, alkenyl, alkynyl; halo; O-alkyl; NH₂; NHMe; N(Me)₂; CN; acyl; aryl; heteroaryl; heterocycle; carbocycle; amino acid residue; or together with the atoms to which they are attached may form a 3-7 membered carbocyclic or heterocyclic ring;

each W is independently O, S, or NR′;

each n is independently 0, 1 or 2;

wherein any branched or unbranched, cyclic or acyclic alkyl, alkenyl, or alkynyl may optionally comprise at least one heteroatom selected from the group consisting of O, S, N and P;

wherein any branched or unbranched, cyclic or acyclic alkyl, alkenyl, alkynyl; aryl; acyl; or aralkyl may optionally be substituted with one or more of OR′, SR′, NR′R″, halogen, NO₂, CN, N₃, CF₃, C(═W)OR′, C(═W)NR′R″, C(═W)SR′, alkyl, alkenyl, alkynyl, aryl, aralkyl, acyl, heterocycle or heteroatom selected from the group consisting of O, S, N and P; and

R is formula (IV) or (V): 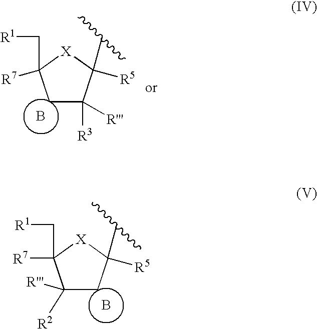
wherein:

R¹, R², R³, R⁵, R⁷, R′″, and [symbol] are all as defined above;

X is O, S, SO₂, CH₂, CHOH, CH-halogen, C-(halogen)₂;

m is 0, 1 or 2; and

all tautomeric, enantiomeric and stereoisomeric forms thereof,

except that R⁵ is OH, NH₂, or SH only when X is C.

In a fifth principal embodiment, a compound selected from the group consisting of Formula (ii), (iv), or (vi), or a pharmaceutically acceptable salt or prodrug thereof, is provided: 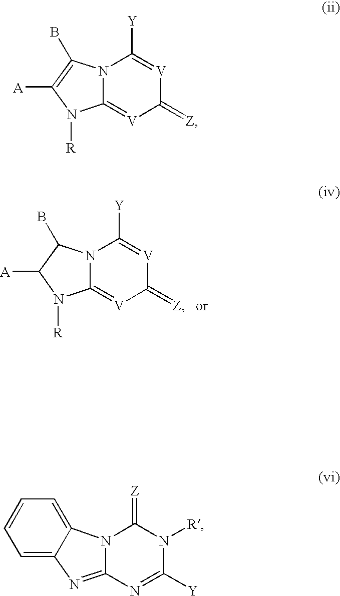
wherein:

A, B and Y, each independently, is H; halogen; OR′, S(O)ₙ; S(O)ₙR′; S(O)ₙR′R″; NR′R″; NR; CN; CF₃; CR′R″; C(═W)OR′; C(═W)SR′; C(═W)NR′R′; C₁₋₄ alkylamino; di(C₁₋₄ alkyl)amino; C₃₋₆ cycloalkylamino; NO₂; N₃; C₁₋₁₀ cyclic or acyclic, branched or unbranched alkyl, alkenyl, alkynyl; aryl; aralkyl; heterocycle; or A and B taken together with the carbon atoms to which they are attached may form a 4-7 membered carbocyclic or heterocyclic ring; with the caveat that A and B may not both be H simultaneously;

Z is O, S, NR′, or CR′R″;

each V is independently N or CR′;

each R′ and R″ independently is H; C₁₋₁₀ cyclic or acyclic, branched or unbranched alkyl, alkenyl, alkynyl; halo; O-alkyl; NH₂; NHMe; N(Me)₂; CN; acyl; aryl; heteroaryl; heterocycle; carbocycle; amino acid residue; or together with the atoms to which they are attached may form a 3-7 membered carbocyclic or heterocyclic ring;

each W is independently O, S, or NR′;

each n is independently 0, 1 or 2;

wherein any branched or unbranched, cyclic or acyclic alkyl, alkenyl, or alkynyl may optionally comprise at least one heteroatom selected from the group consisting of O, S, N and P;

wherein any branched or unbranched, cyclic or acyclic alkyl, alkenyl, alkynyl; aryl; acyl; or aralkyl may optionally be substituted with one or more of OR′, SR′, NR′R″, halogen, NO₂, CN, N₃, CF₃, C(═W)OR′, C(═W)NR′R″, C(═W)SR′, alkyl, alkenyl, alkynyl, aryl, aralkyl, acyl, heterocycle or heteroatom selected from the group consisting of O, S, N and P; and

R is either formula (VI), (VII) or (III): 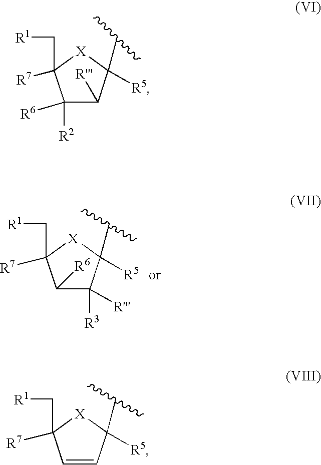
wherein:

R¹, R^{2,} R³, R⁵, R⁶, R⁷, and R′″ are all as defined above;

X is O, S, SO₂, CH₂, CHOH, CH-halogen, C-(halogen)₂;

m is 0, 1 or2; and

all tautomeric, enantiomeric and stereoisomeric forms thereof, except that R⁵ is OH, NH₂, or SH only when X is C.

In a sixth principal embodiment, a compound selected from the Formula (ii), (iv), or (v), or a pharmaceutically acceptable salt or prodrug thereof, is provided: 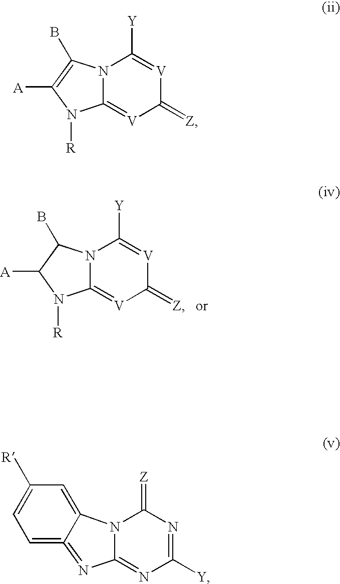
wherein:

A, B and Y, each independently, is H; halogen; OR′, S(O)ₙ; S(O)ₙR′; S(O)ₙR′R″; NR′R″; NR; CN; CF₃; CR′R″; C(═W)OR′; C(═W)SR′; C(═W)NR′R′; C₁₋₄ alkylamino; di(C₁₋₄ alkyl)amino; C₃₋₆ cycloalkylamino; NO₂; N₃; C₁₋₁₀ cyclic or acyclic, branched or unbranched alkyl, alkenyl, alkynyl; aryl; aralkyl; heterocycle; or A and B taken together with the carbon atoms to which they are attached may form a 4-7 membered carbocyclic or heterocyclic ring; with the caveat that A and B may not both be H;

Z is O, S, NR′, or CR′R″;

each V is independently N or CR′;

each R′ and R″ independently is H; C₁₋₁₀ cyclic or acyclic, branched or unbranched alkyl, alkenyl, alkynyl; halo; O-alkyl; NH₂; NHMe; N(Me)₂; CN; acyl; aryl; heteroaryl; heterocycle; carbocycle; amino acid residue; or together with the atoms to which they are attached may form a 3-7 membered carbocyclic or heterocyclic ring;

each W is independently O, S, or NR′;

each n is independently 0, 1 or 2;

wherein any branched or unbranched, cyclic or acyclic alkyl, alkenyl, or alkynyl may optionally comprise at least one heteroatom selected from the group consisting of O, S, N and P;

wherein any branched or unbranched, cyclic or acyclic alkyl, alkenyl, alkynyl; aryl; acyl; or aralkyl may optionally be substituted with one or more of OR′, SR′, NR′R″, halogen, NO₂, CN, N₃, CF₃, C(═W)OR′, C(═W)NR′R″, C(═W)SR′, alkyl, alkenyl, alkynyl, aryl, aralkyl, acyl, heterocycle or heteroatom selected from the group consisting of O, S, N and P; and

R is 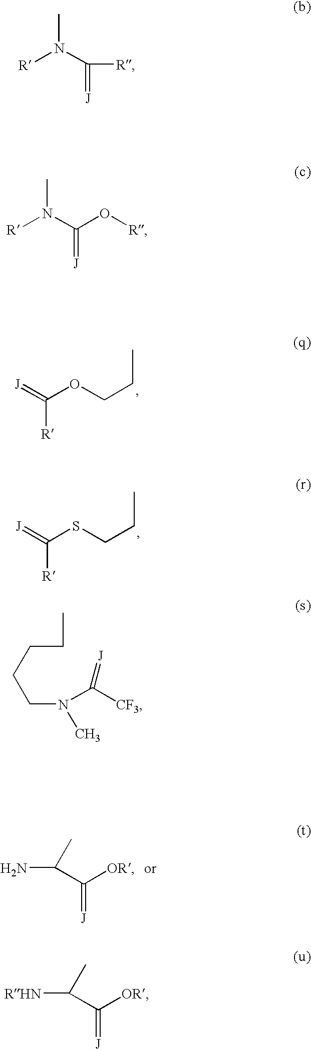
wherein:

each R′ and R″ independently is H; C₁₋₁₀ cyclic or acyclic, branched or unbranched alkyl, alkenyl, alkynyl; halo; O-alkyl; NH₂; NHMe; N(Me)₂; CN; acyl; aryl; heteroaryl; heterocycle; carbocycle; amino acid residue; or together with the atoms to which they are attached may form a 3-7 membered carbocyclic or heterocyclic ring;

J is O, S, or NR′″;

R′″ is H, OH, SH, halo, optionally substituted C₁₋₄ alkyl, optionally substituted C₂₋₄ alkenyl or C₂₋₄ alkynyl, N₃, CN, CH₂CN, CH₂N₃, CH₂NH₂, CH₂NHCH₃, CH₂N(CH₃)₂, CH₂OH, halogenated alkyl, alkoxy, CF₃, C(A′)₃, 2-Br-ethyl, CH₂F, CH₂Cl, CH₂CF₃, CF₂CF₃, CH₂(A′), C(A′)₂(A′)₃, haloalkenyl, Br-vinyl, haloalkynyl; —(CH₂)ₘC(O)OR⁴, —O(acyl), —O(lower acyl), —O(alkyl), —O(lower alkyl), —O(alkenyl), CF₃, halogen, —NO₂, —NH₂, —(CH₂)ₘNHR⁴, —(CH₂)ₘN(R⁴)₂, —(CH₂)ₘC(O)NHR⁴, —(CH₂)ₘC(O)N(R⁴)₂, or C₃₋₇ cycloalkylamino, and where the optional substitutions on alkyl, alkenyl and/or alkynyl may be one or more halogen, hydroxy, amino, alkoxy, or alkylthio groups or atoms taken in any combination; and

all tautomeric, enantiomeric and stereoisomeric forms thereof.

In a seventh principal embodiment, a compound selected from the Formula (ii), (iv), or (vi), or a pharmaceutically acceptable salt or prodrug thereof, is provided: 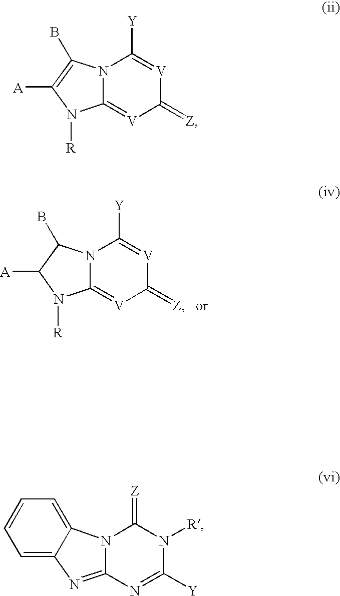
wherein:

A, B and Y, each independently, is H; halogen; OR′, S(O)ₙ; S(O)ₙR′; S(O)ₙR′R″; NR′R″; NR; CN; CF₃; CR′R″; C(═W)OR′; C(═W)SR′; C(═W)NR′R′; C₁₋₄ alkylamino; di(C₁₋₄ alkyl)amino; C₃₋₆ cycloalkylamino; NO₂; N₃; C₁₋₁₀ cyclic or acyclic, branched or unbranched alkyl, alkenyl, alkynyl; aryl; aralkyl; heterocycle; or A and B taken together with the carbon atoms to which they are attached may form a 4-7 membered carbocyclic or heterocyclic ring; with the caveat that A and B may not both be H;

Z is O, S, NR′, or CR′R″;

each V is independently N or CR′;

each R′ and R″ independently is H; C₁₋₁₀ cyclic or acyclic, branched or unbranched alkyl, alkenyl, alkynyl; halo; O-alkyl; NH₂; NHMe; N(Me)₂; CN; acyl; aryl; heteroaryl; heterocycle; carbocycle; amino acid residue; or together with the atoms to which they are attached may form a 3-7 membered carbocyclic or heterocyclic ring;

each W is independently O, S, or NR′;

each n is independently 0, 1 or 2;

wherein any branched or unbranched, cyclic or acyclic alkyl, alkenyl, or alkynyl may optionally comprise at least one heteroatom selected from the group consisting of O, S, N and P;

wherein any branched or unbranched, cyclic or acyclic alkyl, alkenyl, alkynyl; aryl; acyl; or aralkyl may optionally be substituted with one or more of OR′, SR′, NR′R″, halogen, NO₂, CN, N₃, CF₃, C(═W)OR′, C(═W)NR′R″, C(═W)SR′, alkyl, alkenyl, alkynyl, aryl, aralkyl, acyl, heterocycle or heteroatom selected from the group consisting of O, S, N and P; and

R is selected from the group consisting of: 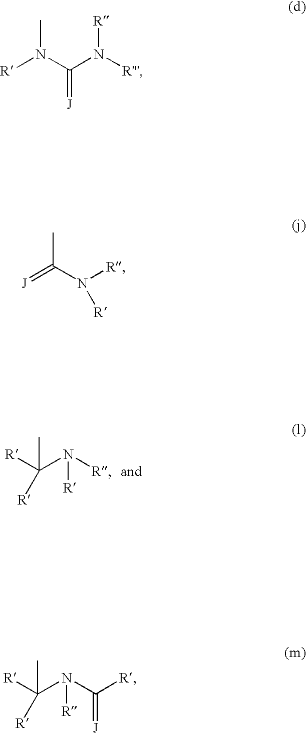
wherein:

each R′ and R″ independently is H; C₁₋₁₀ cyclic or acyclic, branched or unbranched alkyl, alkenyl, alkynyl; halo; O-alkyl; NH₂; NHMe; N(Me)₂; CN; acyl; aryl; heteroaryl; heterocycle; carbocycle; amino acid residue; or together with the atoms to which they are attached may form a 3-7 membered carbocyclic or heterocyclic ring;

J is O, S, or NR′″;

R′″ is H, OH, SH, halo, optionally substituted C₁₋₄ alkyl, optionally substituted C₂₋₄ alkenyl or C₂₋₄ alkynyl, N₃, CN, CH₂CN, CH₂N₃, CH₂NH₂, CH₂NHCH₃, CH₂N(CH₃)₂, CH₂OH, halogenated alkyl, alkoxy, CF₃, C(A′)₃, 2-Br-ethyl, CH₂F, CH₂Cl, CH₂CF₃, CF₂CF₃, CH₂(A′), C(A′)₂(A′)₃, haloalkenyl, Br-vinyl, haloalkynyl; —(CH₂)ₘC(O)OR⁴, —O(acyl), —O(lower acyl), —O(alkyl), —O(lower alkyl), —O(alkenyl), CF₃, halogen, —NO₂, —NH₂, —(CH₂)ₘNHR⁴, —(CH₂)ₘN(R⁴)₂, —(CH₂)ₘC(O)NHR⁴, —(CH₂)ₘC(O)N(R⁴)₂, or C₃₋₇ cycloalkylamino, and where the optional substitutions on alkyl, alkenyl and/or alkynyl may be one or more halogen, hydroxy, amino, alkoxy, or alkylthio groups or atoms taken in any combination; and

all tautomeric, enantiomeric and stereoisomeric forms thereof

In an eighth principal embodiment, a compound selected from the Formula (ii), (iv), or (vi) or a pharmaceutically acceptable salt or prodrug thereof, is provided: 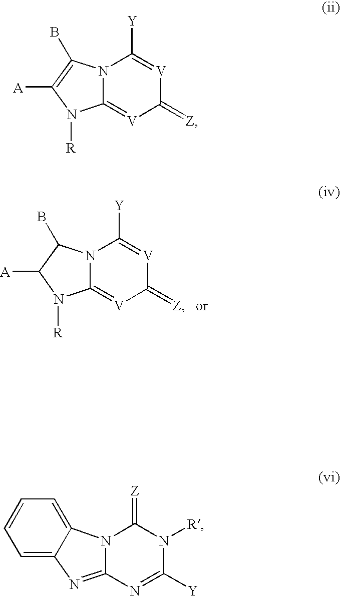
wherein:

A, B and Y, each independently, is H; halogen; OR′, S(O)ₙ; S(O)ₙR′; S(O)ₙR′R″; NR′R″; NR; CN; CF₃; CR′R″; C(═W)OR′; C(═W)SR′; C(═W)NR′R′; C₁₋₄ alkylamino; di(C₁₋₄ alkyl)amino; C₃₋₆ cycloalkylamino; NO₂; N₃; C₁₋₁₀ cyclic or acyclic, branched or unbranched alkyl, alkenyl, alkynyl; aryl; aralkyl; heterocycle; or A and B taken together ay not both be H;

Z is O, S, NR′, or CR′R″;with the carbon atoms to which they are attached may form a 4-7 membered carbocyclic or heterocyclic ring; with the caveat that A and B m

each V is independently N or CR′;

each R′ and R″ independently is H; C₁₋₁₀ cyclic or acyclic, branched or unbranched alkyl, alkenyl, alkynyl; halo; O-alkyl; NH₂; NHMe; N(Me)₂; CN; acyl; aryl; heteroaryl; heterocycle; carbocycle; amino acid residue; or together with the atoms to which they are attached may form a 3-7 membered carbocyclic or heterocyclic ring;

each W is independently O, S, or NR′;

each n is independently 0, 1 or 2;

wherein any branched or unbranched, cyclic or acyclic alkyl, alkenyl, or alkynyl may optionally comprise at least one heteroatom selected from the group consisting of O, S, N and P;

wherein any branched or unbranched, cyclic or acyclic alkyl, alkenyl, alkynyl; aryl; acyl; or aralkyl may optionally be substituted with one or more of OR′, SR′, NR′R″, halogen, NO₂, CN, N₃, CF₃, C(═W)OR′, C(═W)NR′R″, C(═W)SR′, alkyl, alkenyl, alkynyl, aryl, aralkyl, acyl, heterocycle or heteroatom selected from the group consisting of O, S, N and P; and

R is selected from the group consisting of: 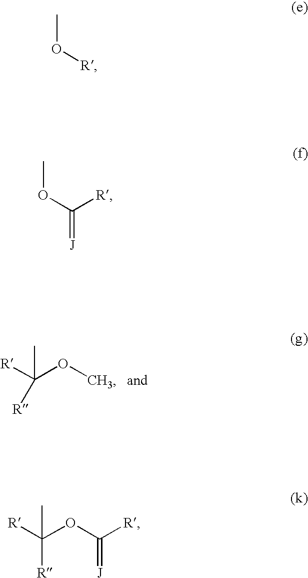
wherein:

each R′ and R″ independently is H; C₁₋₁₀ cyclic or acyclic, branched or unbranched alkyl, alkenyl, alkynyl; halo; O-alkyl; NH₂; NHMe; N(Me)₂; CN; acyl; aryl; heteroaryl; heterocycle; carbocycle; amino acid residue; or together with the atoms to which they are attached may form a 3-7 membered carbocyclic or heterocyclic ring;

J is O, S, or NR′″;

R′″ is H, OH, SH, halo, optionally substituted C₁₋₄ alkyl, optionally substituted C₂₋₄ alkenyl or C₂₋₄ alkynyl, N₃, CN, CH₂CN, CH₂N₃, CH₂NH₂, CH₂NHCH₃, CH₂N(CH₃)₂, CH₂OH, halogenated alkyl, alkoxy, CF₃, C(A′)₃, 2-Br-ethyl, CH₂F, CH₂Cl, CH₂CF₃, CF₂CF₃, CH₂(A′), C(A′)₂(A′)₃, haloalkenyl, Br-vinyl, haloalkynyl; —(CH₂)ₘC(O)OR⁴, —O(acyl), —O(lower acyl), —O(alkyl), —O(lower alkyl), —O(alkenyl), CF₃, halogen, —NO₂, —NH₂, —(CH₂)ₘNHR⁴, —(CH₂)ₘN(R⁴)₂, —(CH₂)ₘC(O)NHR⁴, —(CH₂)ₘC(O)N(R⁴)₂, or C₃₋₇ cycloalkylamino, and where the optional substitutions on alkyl, alkenyl and/or alkynyl may be one or more halogen, hydroxy, amino, alkoxy, or alkylthio groups or atoms taken in any combination; and

all tautomeric, enantiomeric and stereoisomeric forms thereof.

In a ninth principal embodiment, a compound selected from the Formulae (i), (ii), or (v) or a pharmaceutically acceptable salt or prodrug thereof, is provided: 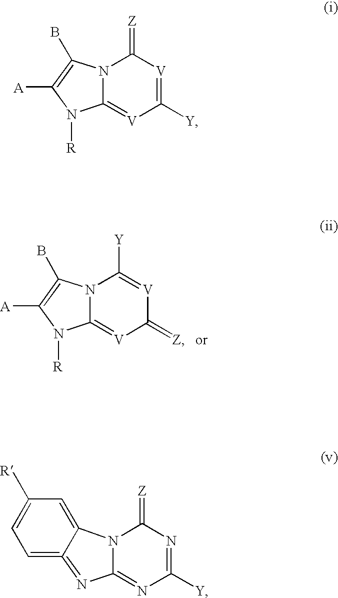
wherein:

A, B and Y, each independently, is H; halogen; OR′, S(O)ₙ; S(O)ₙR′; S(O)ₙR′R″; NR′R″; CN; CF₃; CR′R″; C(═W)OR′; C(═W)SR′; C(═W)NR′R′; C₁₋₄ alkylamino; di(C₁₋₄ alkyl)amino; C₃₋₆ cycloalkylamino; NO₂; N₃; C₁₋₁₀ cyclic or acyclic, branched or unbranched alkyl, alkenyl, alkynyl; aryl; aralkyl; heterocycle; or A and B taken together with the carbon atoms to which they are attached may form a 4-7 membered carbocyclic or heterocyclic ring; with the caveat that A and B may not both be H;

Z is O, S, NR′, or CR′R″;

each V is independently N or CR′;

each R′ and R″ independently is H; C₁₋₁₀ cyclic or acyclic, branched or unbranched alkyl, alkenyl, alkynyl; halo; O-alkyl; NH₂; NHMe; N(Me)₂; CN; acyl; aryl; heteroaryl; heterocycle; carbocycle; amino acid residue; or together with the atoms to which they are attached may form a 3-7 membered carbocyclic or heterocyclic ring;

each W is independently O, S, or NR′;

each n is independently 0, 1 or 2;

wherein any branched or unbranched, cyclic or acyclic alkyl, alkenyl, or alkynyl may optionally comprise at least one heteroatom selected from the group consisting of O, S, N and P;

wherein any branched or unbranched, cyclic or acyclic alkyl, alkenyl, alkynyl; aryl; acyl; or aralkyl may optionally be substituted with one or more of OR′, SR′, NR′R″, halogen, NO₂, CN, N₃, CF₃, C(═W)OR′, C(═W)NR′R″, C(═W)SR′, alkyl, alkenyl, alkynyl, aryl, aralkyl, acyl, heterocycle or heteroatom selected from the group consisting of O, S, N and P; and

R is selected from the group consisting of: 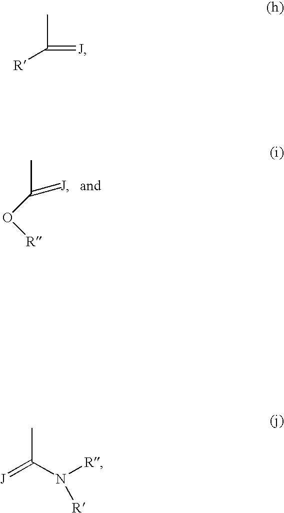
wherein:

each R′ and R″ independently is H; C₁₋₁₀ cyclic or acyclic, branched or unbranched alkyl, alkenyl, alkynyl; halo; O-alkyl; NH₂; NHMe; N(Me)₂; CN; acyl; aryl; heteroaryl; heterocycle; carbocycle; amino acid residue; or together with the atoms to which they are attached may form a 3-7 membered carbocyclic or heterocyclic ring;

J is O, S, or NR′″;

R′″ is H, OH, SH, halo, optionally substituted C₁₋₄ alkyl, optionally substituted C₂₋₄ alkenyl or C₂₋₄ alkynyl, N₃, CN, CH₂CN, CH₂N₃, CH₂NH₂, CH₂NHCH₃, CH₂N(CH₃)₂, CH₂OH, halogenated alkyl, alkoxy, CF₃, C(A′)₃, 2-Br-ethyl, CH₂F, CH₂Cl, CH₂CF₃, CF₂CF₃, CH₂(A′), C(A′)₂(A′)₃, haloalkenyl, Br-vinyl, haloalkynyl; —(CH₂)ₘC(O)OR⁴, —O(acyl), —O(lower acyl), —O(alkyl), —O(lower alkyl), —O(alkenyl), CF₃, halogen, —NO₂, —NH₂, —(CH₂)ₘNHR⁴, —(CH₂)ₘN(R⁴)₂, —(CH₂)ₘC(O)NHR⁴, —(CH₂)ₘC(O)N(R⁴)₂, or C₃₋₇ cycloalkylamino, and where the optional substitutions on alkyl, alkenyl and/or alkynyl may be one or more halogen, hydroxy, amino, alkoxy, or alkylthio groups or atoms taken in any combination; and

all tautomeric, enantiomeric and stereoisomeric forms thereof.

In a tenth principal embodiment, a compound selected from the group consisting of Formulae (i), (ii), (iii), (iv), (v) or (vi), or a pharmaceutically acceptable salt or prodrug thereof, is provided: 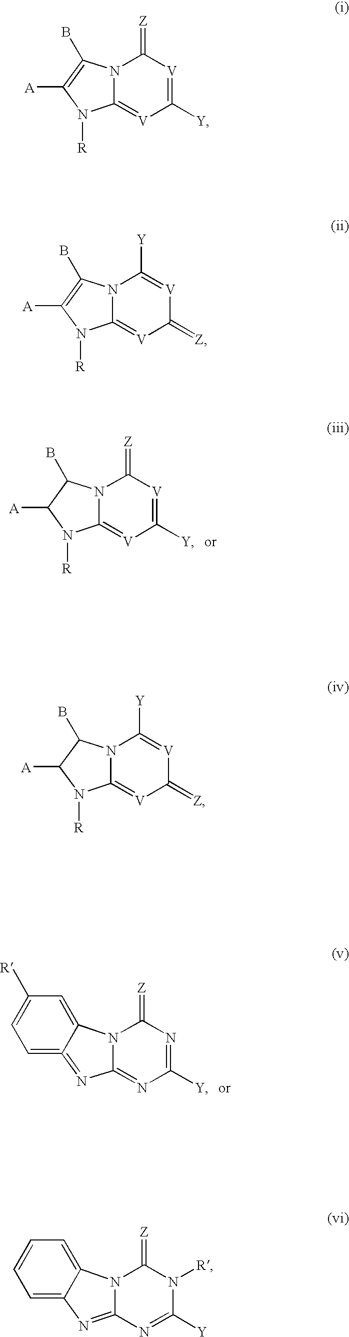
wherein:

A, B and Y, each independently, is H; halogen; OR′, S(O)ₙ; S(O)ₙR′; S(O)ₙR′R″; NR′R″; NR; CN; CF₃; CR′R″; C(═W)OR′; C(═W)SR′; C(═W)NR′R′; C₁₋₄ alkylamino; di(C₁₋₄ alkyl)amino; C₃₋₆ cycloalkylamino; NO₂; N₃; C₁₋₁₀ cyclic or acyclic, branched or unbranched alkyl, alkenyl, alkynyl; aryl; aralkyl; heterocycle; or A and B taken together with the carbon atoms to which they are attached may form a 4-7 membered carbocyclic or heterocyclic ring; with the caveat that A and B may not both be H;

Z is O, S, NR′, or CR′R″;

each V is independently N or CR′;

each R′ and R″ independently is H; C₁₋₁₀ cyclic or acyclic, branched or unbranched alkyl, alkenyl, alkynyl; halo; O-alkyl; NH₂; NHMe; N(Me)₂; CN; acyl; aryl; heteroaryl; heterocycle; carbocycle; amino acid residue; or together with the atoms to which they are attached may form a 3-7 membered carbocyclic or heterocyclic ring;

each W is independently O, S, or NR′;

each n is independently 0, 1 or 2;

wherein any branched or unbranched, cyclic or acyclic alkyl, alkenyl, or alkynyl may optionally comprise at least one heteroatom selected from the group consisting of O, S, N and P;

wherein any branched or unbranched, cyclic or acyclic alkyl, alkenyl, alkynyl; aryl; acyl; or aralkyl may optionally be substituted with one or more of OR′, SR′, NR′R″, halogen, NO₂, CN, N₃, CF₃, C(═W)OR′, C(═W)NR′R″, C(═W)SR′, alkyl, alkenyl, alkynyl, aryl, aralkyl, acyl, heterocycle or heteroatom selected from the group consisting of O, S, N and P; and

R is: 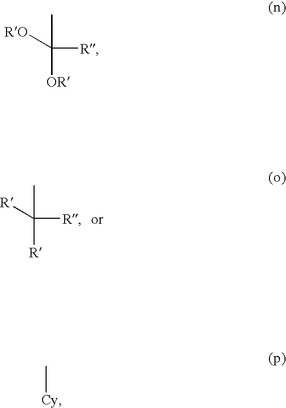
wherein:

each R′ and R″ independently is H; C₁₋₁₀ cyclic or acyclic, branched or unbranched alkyl, alkenyl, alkynyl; halo; O-alkyl; NH₂; NHMe; N(Me)₂; CN; acyl; aryl; heteroaryl; heterocycle; carbocycle; amino acid residue; or together with the atoms to which they are attached may form a 3-7 membered carbocyclic or heterocyclic ring;

Cy is any cyclic structure, including a carbocycle, heterocycle or heteroaryl;

J is O, S, or NR′″;

R′″ is H, OH, SH, halo, optionally substituted C₁₋₄ alkyl, optionally substituted C₂₋₄ alkenyl or C₂₋₄ alkynyl, N₃, CN, CH₂CN, CH₂N₃, CH₂NH₂, CH₂NHCH₃, CH₂N(CH₃)₂, CH₂OH, halogenated alkyl, alkoxy, CF₃, C(A′)₃, 2-Br-ethyl, CH₂F, CH₂Cl, —(CH₂)ₘC(O)OR⁴, —O(acyl), —O(lower acyl), —O(alkyl), —O(lower alkyl), —O(alkenyl), CF₃, halogen, —NO₂, —NH₂, —(CH₂)ₘNHR⁴, —(CH₂)ₘN(R⁴)₂, —(CH₂)ₘC(O)NHR⁴, —(CH₂)ₘC(O)N(R⁴)₂, or C₃₋₇ cycloalkylamino, and where the optional substitutions on alkyl, alkenyl and/or alkynyl may be one or more halogen, hydroxy, amino, alkoxy, or alkylthio groups or atoms taken in any combination; and

all tautomeric, enantiomeric and stereoisomeric forms thereof.

In all embodiments, any optional substituents may be selected that do not adversely affect the properties of the molecule, and for example, may be selected from the group consisting of one or more halogen, amino, hydroxy, carboxy and alkoxy groups or atoms, among others. It is to be understood that all stereoisomeric and tautomeric forms of the compounds shown are included herein.

### Nucleotide Prodrug Formulations

Any of the nucleoside analogs or nucleosides described herein can be administered as a prodrug to increase the activity, bioavailability, stability or otherwise alter the properties of the biologically active compound. Any nucleoside analog group that easily dissociates from the base formulae (i)-(vi) may be considered to be a prodrug form of the active compound. For example, a diethylacetyl group bonded at position 9 of an optionally substituted 5-aza-7-deazapurine base could undergo hydrolysis to produce the active free base. In general, alkylation, acylation or other lipophilic modification of the mono-, di- or triphosphate of a nucleoside reduces polarity and allows passage into cells. Examples of substituent groups that can replace one or more hydrogens on the phosphate moiety are alkyl, aryl, steroids, carbohydrates, including sugars, 1,2-diacylglycerol and alcohols. Many are described in R. Jones and N. Bischoferger, Antiviral Research, 1995, 27:1-17. Any of these can be used in combination with Formulae (I)-(VIII) as disclosed herein to achieve a desired effect.

In cases where compounds are sufficiently basic or acidic to form stable nontoxic acid or base salts, administration of the compound as a pharmaceutically acceptable salt may be appropriate. Examples of pharmaceutically acceptable salts are organic acid addition salts formed with acids, which form a physiological acceptable anion, for example, tosylate, methanesulfonate, acetate, citrate, malonate, tartarate, succinate, benzoate, ascorate, α-ketoglutarate, and α-glycerophosphate. Suitable inorganic salts may also be formed, including, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

If a nucleoside of formulae (I)-(VIII) is the active compound of choice, it can also be provided as a 5′-phosphoether lipid or a 5′-ether lipid, as disclosed in the following references, which are incorporated by reference herein: Kucera, L. S., N. Iyer, E. Leake, A. Raen, Modest E. K., D. L. W., and C. Piantadosi. 1990. “Novel membrane-interactive ether lipid analogs that inhibit infectious HIV-1 production and induce defective virus formation.” AIDS Res. Hum. Retro Viruses. 6:491-501; Piantadosi, C., J. Marasco C. J., S. L. Morris-Natschke, K. L. Meyer, F. Gumus, J. R. Surles, K. S. Ishaq, L. S. Kucera, N. Iyer, C. A. Wallen, S. Piantadosi, and E. J. Modest. 1991. “Synthesis and evaluation of novel ether lipid nucleoside conjugates for anti-HIV activity.” J. Med. Chem. 34:1408.1414; Hosteller, K. Y., D. D. Richman, D. A. Carson, L. M. Stuhmiller, G. M. T. van Wijk, and H. van den Bosch., 1992. “Greatly enhanced inhiition of human immunodeficiency virus type 1 replication in CEM and HT4-6C cells by 3′-deoxythymidine diphosphate dimyristoylglycerol, a lipid prodrug of 3-deoxythymidine.” Antimicro. Agents Chemother. 36:2025.2029; Hosetler, K. Y., L. M. Stuhmiller, H. Lenting, H. van den Bosch, and D. D. Richman, 1990. “Synthesis and antiretroviral activity of phospholipid analogs of azidothymidine and other antiviral nucleosides.” J. Biol. Chem. 265:61127.

Nonlimiting examples of U.S. patents that disclose suitable lipophilic substituents that can be covalently incorporated into a nucleoside, typically at the 5′-OH position of the nucleoside or lipophilic preparations, include U.S. Pat. No. 5,149,794 (Sep. 22, 1992, Yatvin et al.); U.S. Pat. No. 5,194,654 (Mar. 16, 1993, Hostetler et al., U.S. Pat. No. 5,223,263 (June 29, 1993, Hostetler et al.); U.S. Pat. No. 5,256,641 (Oct. 26, 1993, Yatvin et al.); U.S. Pat. No. 5,411,947 (May 2, 1995, Hostetler et al.); U.S. Pat. No. 5,463,092 (Oct. 31, 1995, Hostetler et al.); U.S. Pat. No. 5,543,389 (Aug. 6, 1996, Yatvin et al.); U.S. Pat. No. 5,543,390 (Aug. 6, 1996, Yatvin et al.); U.S. Pat. No. 5,543,391 (Aug. 6, 1996, Yatvin et al.); and U.S. Pat. No. 5,554,728 (Sep. 10, 1996; Basava et al.), all of which are incorporated herein by reference. Foreign patent applications that disclose lipophilic substituents that can be attached to the nucleosides of the present invention, or lipophilic preparations, include WO 89/02733, WO 90/00555, WO 91/16920, WO 91/18914, WO 93/00910, WO 94/26273, WO 96/15132, EP 0 350 287, EP 93917054.4, and WO 91/19721. In another embodiment, the nucleoside is a 5′ phosphonate.

### Methods of Treatment

In one embodiment of the invention, a method of treatment or prophylaxis of a host infected with, or at risk for infection with, a flavivirus is provided that includes administering an antivirally or treatment effective amount of a compound of the invention to the host, optionally in combination or alternation or sequentially with another antiviral agent. In a particular embodiment, a method of treatment of a host infected with a hepatitis C virus is provided. In another embodiment, the use of a compound of the invention for the treatment of a host infected with a flavivirus, and particularly hepatitis C is provided. In yet another embodiment, the use of a compound of the invention in the manufacture of a medicament for the treatment of a host infected with a flavivirus, and particularly hepatitis C is provided.

The active compounds of the present invention can be administered in combination, alternation or sequential steps with another anti-HCV agent. In combination therapy, effective dosages of two or more agents are administered together, whereas in alternation or sequential-step therapy, an effective dosage of each agent is administered serially or sequentially. The dosages given will depend on absorption, inactivation and excretion rates of the drug as well as other factors known to those of skill in the art. It is to be noted that dosage values will also vary with the severity of the condition to be alleviated. It is to be further understood that for any particular subject, specific dosage regimens and schedules should be adjusted over time according to the individual need and the professional judgment of the person administering or supervising the administration of the compositions. In some embodiments, an anti-HCV (anti-pestivirus or anti-flavivirus) compound that exhibits an EC₅₀ of 10-15 μM, or typically less than 1-5 μM, is desirable.

The active compound can be administered as any salt or prodrug that upon administration to the recipient is capable of providing directly or indirectly the parent compound, or that exhibits activity itself. Nonlimiting examples are the pharmaceutically acceptable salts, which are alternatively referred to as “physiologically acceptable salts”, and a compound that has been alkylated or acylated at the 5′-position or on the purine or pyrimidine base, thereby forming a type of “pharmaceutically acceptable prodrug”. Further, the modifications can affect the biological activity of the compound, in some cases increasing the activity over the parent compound. This can easily be assessed by preparing the salt or prodrug and testing its antiviral activity according to the methods described herein, or other methods known to those skilled in the art.

Flaviviruses included within the scope of this invention are discussed generally in Fields Virology, Editors: Fields, .N., Knipe, D. M. and Howley, P. M.; Lippincott-Raven Pulishers, Philadelphia, Pa.; Chapter 31 (1996). Specific flaviviruses include, without limitation: Absettarov; Alfuy; Apoi; Aroa; Bagaza; Banzi; Bououi; Bussuquara; Cacipacore; Carey Island; Dakar bat; Dengue viruses 1, 2, 3 and 4; Edge Hill; Entebbe bat; Gadgets Gully; Hanzalova; Hypr; Ilheus; Israel turkey meningoencephalitis; Japanese encephalitis; Jugra; Jutiapa; Kadam; Karshi; Kedougou; Kokoera; Koutango; Kumlinge; Kunjin; Kyasanur Forest disease; Langat; Louping ill; Meaban; Modoc; Montana myotis leukoencephalitis; Murray valley encephalitis; Naranjal; Negishi; Ntaya; Omsk hemorrhagic fever; Phnom-Penh bat; Powassan; Rio Bravo; Rocio; Royal Farm; Russian spring-summer encephalitis; Saboya; St. Louis encephalitis; Sal Vieja; San Perlita; Saumarez Reef; Sepik; Sokuluk; Spondweni; Stratford; Temusu; Tyuleniy; Uganda S, Usutu, Wesselsbron; West Nile; Yaounde; Yellow fever; and Zika.

Pestiviruses included within the scope of this invention are also discussed generally in Fields Virology (Wd.). Specific pestiviruses include, without limitation: bovine viral diarrhea virus (“VDV”); classical swine fever virus (“CSFV”) also known as hog cholera virus); and border disease virus (“DV”).

### Combination and Alternation Therapy

Drug-resistant variants of flaviviruses, pestiviruses or HCV are known to emerge after prolonged treatment with an antiviral agent. Drug resistance most typically occurs by mutation of a gene that encodes for an enzyme used in viral replication. The efficacy of a drug against the viral infection can be prolonged, augmented, or restored by administering the compound in combination or alternation with a second, and perhaps third, antiviral compound that induces a different mutation from that caused by the principle drug. Alternatively, the pharmacokinetics, biodistriution or other parameter of the drug can be altered by such combination or alternation therapy. In general, combination therapy is typical rather than alternation therapy because it induces multiple simultaneous stresses on the virus.

Any of the viral treatments described in the Background of the Invention can be used in combination or alternation with the compounds described in this specification. Nonlimiting examples include:
(1) an interferon and/or ribavirin (see, for example, Battaglia, A. M. et al., Ann. Pharmacother. 34:487-494, 2000); Berenguer, M. et al. Antivir. Ther. 3(Suppl. 3):125-136, 1998);
(2) Substrate-based NS3 protease inhibitors (see, for example, Attwood et al., Antiviral peptide derivatives, PCT WO 98/22496, 1998; Attwood et al., Antiviral Chemistry and Chemotherapy 1999, 10, 259-273; Attwood et al., Preparation and use of amino acid derivatives as anti-viral agents, German Patent Pub. DE 19914474; Tung et al. Inhibitors of serine proteases, particularly hepatitis C virus NS3 protease, PCT WO 98/17679), including alphaketoamides and hydrazinoureas, and inhibitors that terminate in an electrophile such as a boronic acid or phosphonate (see, for example, Llinas-Brunet et al, Hepatitis C inhibitor peptide analogues, PCT WO 99/07734).
(3) Non-substrate-based inhibitors such as 2,4,6-trihydroxy-3-nitro-benzamide derivatives (see, for example, Sudo K. et al., Biochemical and Biophysical Research Communications, 1997, 238, 643-647; Sudo K. et al. Antiviral Chemistry and Chemotherapy, 1998, 9, 186), including RD3-4082 and RD3-4078, the former substituted on the amide with a 14 carbon chain and the latter processing a para-phenoxyphenyl group;
(4) Thiazolidine derivatives, for example, that show relevant inhibition in a reverse-phase HPLC assay with an NS3/4A fusion protein and NS5A/5B substrate (see, for example, Sudo K. et al., Antiviral Research, 1996, 32, 9-18), especially compound RD-1-6250, possessing a fused cinnamoyl moiety substituted with a long alkyl chain, RD4 6205 and RD4 6193;
(5) Thiazolidines and benzanilides, for example, as identified in Kakiuchi N. et al. J. EBS Letters 421, 217-220; Takeshita N. et al. Analytical Biochemistry, 1997, 247, 242-246;
(6) A phenanthrenequinone possessing activity against protease in a SDS-PAGE and autoradiography assay, for example, isolated from the fermentation culture broth of Streptomyces sp., Sch 68631 (see, for example, Chu M. et al., Tetrahedron Letters, 1996, 37, 7229-7232), and Sch 351633, isolated from the fungus Penicillium griseofulvum, which demonstrates activity in a scintillation proximity assay (see, for example, Chu M. et al., Bioorganic and Medicinal Chemistry Letters 9, 1949-1952);
(7) Selective NS3 inhibitors, for example, based on the macromolecule elgin c, isolated from leech (see, for example, Qasim M. A. et al., Biochemistry, 1997, 36, 1598-1607);
(8) Helicase inhibitors (see, for example, Diana G. D. et al., Compounds, compositions and methods for treatment of hepatitis C, U.S. Pat. No. 5,633,358; Diana G. D. et al., Piperidine derivatives, pharmaceutical compositions thereof and their use in the treatment of hepatitis C, PCT WO 97/36554);
(9) Polymerase inhibitors such as
   i) nucleotide analogues, for example, gliotoxin (see, for example, Ferrari R. et al. Journal of Virology, 1999, 73, 1649-1654);
   ii) the natural product cerulenin (see, for example, Lohmann V. et al., Virology, 1998, 249, 108-118); and
   iii) non-nucleoside polymerase inhibitors, including, for example, compound R803 (see, for example, WO 04/018463 A2 and WO 03/040112 A1, both to Rigel Pharmaceuticals, Inc.); substituted diamine pyrimidines (see, for example, WO 03/063794 A2 to Rigel Pharmaceuticals, Inc.);

benzimidazole derivatives (see, for example, Bioorg. Med. Chem. Lett., 2004, 14:119-124 and Bioorg. Med. Chem. Lett., 2004, 14:967-971, both to Boehringer Ingelheim Corporation); N,N-disubstituted phenylalanines (see, for example, J. Biol. Chem., 2003, 278:9495-98 and J. Med. Chem., 2003, 13:1283-85, both to Shire Biochem, Inc.); substituted thiophene-2-carboxylic acids (see, for example, Bioorg. Med. Chem. Lett., 2004, 14:793-796 and Bioorg. Med. Chem. Lett., 2004, 14:797-800, both to Shire Biochem, Inc.); α,γ-diketoacids (see, for example, J. Med. Chem., 2004, 14-17 and WO 00/006529 A1, both to Merck & Co., Inc.); and meconic acid derivatives (see, for example, Bioorg. Med. Chem. Lett., 2004, 3257-3261, WO 02/006246 A1 and W003/062211 A1, all to IRBM Merck & Co., Inc.);
(10) Antisense phosphorothioate oligodeoxynucleotides (S-ODN) complementary, for example, to sequence stretches in the 5′ non-coding region (NCR) of the virus (see, for example, Alt M. et al., Hepatology, 1995, 22, 707-717), or to nucleotides 326-348 comprising the 3′ end of the NCR and nucleotides 371-388 located in the core coding region of the HCV RNA (see, for example, Alt M. et al., Archives of Virology, 1997, 142, 589-599; Galderisi U. et al., Journal of Cellular Physiology, 1999, 181, 251-257).
(11) Inhibitors of IRES-dependent translation (see, for example, Ikeda N et al., Agent for the prevention and treatment of hepatitis C, Japanese Patent Pub. JP-08268890; Kai Y. et al. Prevention and treatment of viral diseases, Japanese Patent Pub. JP-10101591).
(12) Nuclease-resistant ribozymes (see, for example, Maccjak, D. J. et al., Hepatology 1999, 30, abstract 995).
(13) Nucleoside analogs have also been developed for the treatment of Flaviviridae infections. Examples include the following.

Idenix Pharmaceuticals, Ltd. discloses branched nucleosides, and their use in the treatment of HCV and flaviviruses and pestiviruses in U.S. Pat. No. 6,914,054, which will issue on Jul. 5, 2005, and U.S. Pat. No. 6,812,219, issued Nov. 2, 2004, which correspond to International Publication Nos. WO 01/90121 and WO 01/92282. A method for the treatment of hepatitis C infection (and flaviviruses and pestiviruses) in humans and other host animals is disclosed in the Idenix publications that includes administering an effective amount of a biologically active 1′, 2′, 3′ or 4′-branched β-D or β-L nucleosides or a pharmaceutically acceptable salt or prodrug thereof, administered either alone or in combination, optionally in a pharmaceutically acceptable carrier. See also U.S. patent Publication Nos. 2004/0006002 and 2004/0006007 as well as WO 03/026589 and WO 03/026675. Idenix Pharmaceuticals, Ltd. also discloses in U.S. patent Publication No. 2004/0077587 pharmaceutically acceptable branched nucleoside prodrugs, and their use in the treatment of HCV and flaviviruses and pestiviruses in prodrugs. See also PCT Publication Nos. WO 04/002422, WO 04/002999, WO 04/003000; WO 04/024095 and WO 05/009418.

Biota Inc. discloses various phosphate derivatives of nucleosides, including 1′, 2′, 3′ or 4′-branched β-D or β-L nucleosides, for the treatment of hepatitis C infection in International Patent Publication WO 03/072757.

Emory University and the University of Georgia Research Foundation, Inc. (UGARF) discloses the use of 2′-fluoronucleosides for the treatment of HCV in U.S. Pat. No. 6,348,587. See also U.S. patent Publication No. 2002/0198171 and International Patent Publication WO 99/43691.

BioChem Pharma Inc. (now Shire Biochem, Inc.) discloses the use of various 1,3-dioxolane nucleosides for the treatment of a Flaviviridae infection in U.S. Pat. No. 6,566,365. See also U.S. Pat. Nos. 6,340,690 and 6,605,614; U.S. patent Publication Nos. 2002/0099072 and 2003/0225037, as well as International Publication No. WO 01/32153 and WO 00/50424.

BioChem Pharma Inc. (now Shire Biochem, Inc.) also discloses various other 2′-halo, 2′-hydroxy and 2′-alkoxy nucleosides for the treatment of a Flaviviridae infection in U.S. patent Publication No. 2002/0019363 as well as International Publication No. WO 01/60315 (PCT/CA01/00197; filed Feb. 19, 2001).

ICN Pharmaceuticals, Inc. discloses various nucleoside analogs that are useful in modulating immune response in U.S. Pat. Nos. 6,495,677 and 6,573,248. See also WO 98/16184, WO 01/68663, and WO 02/03997.

U.S. Pat. No. 6,660,721; U.S. patent Publication Nos. 2003/083307 A1, 2003/008841 A1, and 2004/0110718; as well as International Patent Publication Nos. WO 02/18404; WO 02/100415, WO 02/094289, and WO 04/043159; filed by F. Hoffmann-La Roche A G, discloses various nucleoside analogs for the treatment of HCV RNA replication.

Pharmasset Limited discloses various nucleosides and antimetabolites for the treatment of a variety of viruses, including Flaviviridae, and in particular HCV, in U.S. patent Publication Nos. 2003/0087873, 2004/0067877, 2004/0082574, 2004/0067877, 2004/002479, 2003/0225029, and 2002/00555483, as well as International Patent Publication Nos. WO 02/32920, WO 01/79246, WO 02/48165, WO 03/068162, WO 03/068164 and WO 2004/013298.

Merck & Co., Inc. and Isis Pharmaceuticals disclose in U.S. patent Publication No. 2002/0147160, 2004/0072788, 2004/0067901, and 2004/0110717; as well as the corresponding International Patent Publication Nos. WO 02/057425 (PCT/US02/01531; filed Jan. 18, 2002) and WO 02/057287 (PCT/US02/03086; filed Jan. 18, 2002) various nucleosides, and in particular several pyrrolopyrimidine nucleosides, for the treatment of viruses whose replication is dependent upon RNA-dependent RNA polymerase, including Flaviviridae, and in particular HCV. See also WO 2004/000858, WO 2004/003138, WO 2004/007512, and WO 2004/009020.

U.S. patent Publication No. 2003/028013 A1 as well as International Patent Publication Nos. WO 03/051899, WO 03/061576, WO 03/062255 WO 03/062256, WO 03/062257, and WO 03/061385, filed by Ribapharm, also are directed to the use of certain nucleoside analogs to treat hepatitis C virus.

Genelabs Technologies disclose in U.S. patent Publication No. 2004/0063658 as well as International Patent Publication Nos. WO 03/093290 and WO 04/028481 various base modified derivatives of nucleosides, including 1′, 2′, 3′ or 4′-branched β-D or β-L nucleosides, for the treatment of hepatitis C infection.
(14) Other miscellaneous compounds including 1-amino-alkylcyclohexanes (for example, U.S. Pat. No. 6,034,134 to Gold et al.), alkyl lipids (for example, U.S. Pat. No. 5,922,757 to Chojkier et al.), vitamin E and other antioxidants (for example, U.S. Pat. No. 5,922,757 to Chojkier et al.), squalene, amantadine, bile acids (for example, U.S. Pat. No. 5,846,964 to Ozeki et al.), N-(phosphonoacetyl)-L-aspartic acid (for example, U.S. Pat. No. 5,830,905 to Diana et al.), benzenedicarboxamides (for example, U.S. Pat. No. 5,633,388 to Diana et al.), polyadenylic acid derivatives (for example, U.S. Pat. No. 5,496,546 to Wang et al.), 2′,3′-dideoxyinosine (for example, U.S. Pat. No. 5,026,687 to Yarchoan et al.), and benzimidazoles (for example, U.S. Pat. No. 5,891,874 to Colacino et al.).
(15) Other compounds currently in clinical development for treatment of hepatitis c virus include, for example: Interleukin-10 by Schering-Plough, IP-501 by Interneuron, Merimebodib VX-497 by Vertex, AMANTADINE (Symmetrel) by Endo Labs Solvay, HEPTAZYME by RPI, IDN-6556 by Idun Pharma., XTL-002 by XTL., HCV/MF59 by Chiron, CIVACIR by NABI, LEVOVIRIN by ICN, VIRAMIDINE by ICN, ZADAXIN (thymosin alfa-1) by Sci Clone, CEPLENE (histamine dihydrochloride) by Maxim, VX 950/LY 570310 by Vertex/Eli Lilly, ISIS 14803 by Isis Pharmaceutical/Elan, IDN-6556 by Idun Pharmaceuticals, Inc. and JTK 003 by AKROS Pharma.

### Pharmaceutical Compositions

Hosts, including humans, infected with pestivirus, flavivirus, HCV or another organism replicating through a RNA-dependent RNA viral polymerase, can be treated by administering to the patient an effective amount of the active compound or a pharmaceutically acceptable prodrug or salt thereof in the presence of a pharmaceutically acceptable carrier or diluent. The active materials can be administered by any appropriate route, for example, orally, parenterally, intravenously, intradermally, subcutaneously, or topically, in liquid or solid form.

Any dose which achieves the desired results or shows efficacy is appropriate. An exemplary dose of the compound for pestivirus, flavivirus or HCV will be in the range from about 1 to 50 mg/kg, typically 1 to 20 mg/kg, of body weight per day, more generally 0.1 to about 100 mg per kilogram body weight of the recipient per day. The effective dosage range of the pharmaceutically acceptable salts and prodrugs can be calculated based on the weight of the parent compound to be delivered. If the salt or prodrug exhibits activity in itself, the effective dosage can be estimated as above using the weight of the salt or prodrug, or by other means known to those skilled in the art.

The compound is conveniently administered in unit any suitable dosage form, including but not limited to one containing 7 to 3000 mg, typically 70 to 1400 mg of active ingredient per unit dosage form. An oral dosage of 50-1000 mg is usually convenient.

Ideally the active ingredient should be administered to achieve peak plasma concentrations of the active compound of from about 0.2 to 70 μM, typically about 1.0 to 10 μM. This may be achieved, for example, by the intravenous injection of a 0.1 to 5% solution of the active ingredient, optionally in saline, or administered as a bolus of the active ingredient.

The concentration of active compound in the drug composition will depend on absorption, inactivation and excretion rates of the drug as well as other factors known to those of skill in the art. Dosage values will also vary with the severity of the condition to be alleviated. Further, for any particular subject, specific dosage regimens should be adjusted over time according to the individual need and the professional judgment of the person administering or supervising the administration of the compositions, and concentration ranges set forth herein are exemplary only and are not intended to limit the scope or practice of the claimed composition. The active ingredient may be administered at once, or may be divided into a number of smaller doses to be administered at varying intervals of time.

A typical mode of administration of the active compound is oral. Oral compositions will generally include an inert diluent or an edible carrier. They may be enclosed in gelatin capsules or compressed into tablets. For the purpose of oral therapeutic administration, the active compound can be incorporated with excipients and used in the form of tablets, troches, or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or other enteric agents.

The compound can be administered as a component of an elixir, suspension, syrup, wafer, chewing gum or the like. A syrup may contain, in addition to the active compounds, sucrose as a sweetening agent and certain preservatives, dyes and colorings and flavors.

The compound or a pharmaceutically acceptable prodrug or a salt thereof can also be mixed with other active materials that do not impair the desired action, or with materials that supplement the desired action, such as antibiotics, antifungals, anti-inflammatories, or other antivirals, including other nucleoside compounds. Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation can be enclosed in ampoules, disposable syringes or multiple dose vials made of glass or plastic.

If administered intravenously, commonly used carriers are physiological saline or phosphate buffered saline (PBS).

In one embodiment, the active compounds are prepared with carriers that will protect the compound against rapid elimination from the body, such as a controlled release formulation, including implants and microencapsulated delivery systems. biodegradable, biocompatible polymers can be used, such as ethylene vinyl acetate, polyanhydrides, polyglycolic acid, collagen, polyorthoesters and polylactic acid. Methods for preparation of such formulations will be apparent to those skilled in the art. The materials can also be obtained commercially from Alza Corporation.

Liposomal suspensions (including liposomes targeted to infected cells with monoclonal antibodies to viral antigens) are also commonly used as pharmaceutically acceptable carriers. These may be prepared according to methods known to those skilled in the art, for example, as described in U.S. Pat. No. 4,522,811 (which is incorporated herein by reference in its entirety). For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound or its monophosphate, diphosphate, and/or triphosphate derivatives is then introduced into the container. The container is then swirled by hand to free lipid material from the sides of the container and to disperse lipid aggregates, thereby forming the liposomal suspension.

### Processes for the Preparation of Active Compounds

The compounds of the present invention can be synthesized by any means known in the art.

Nucleoside Analogues

The synthesis of nucleoside analogs generally can be achieved by condensation reactions that utilize sodium hydride and either an alkyl halide or epoxide containing the “R” group of interest. “IV” groups of interest include but are not limited to: 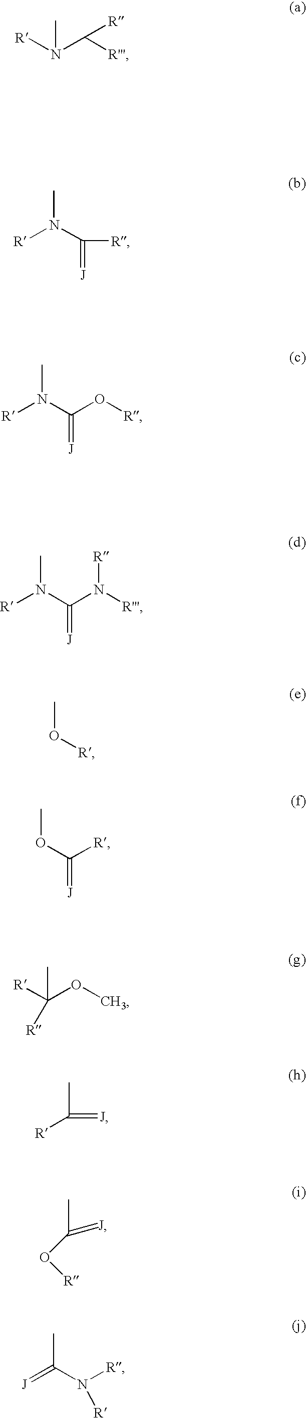 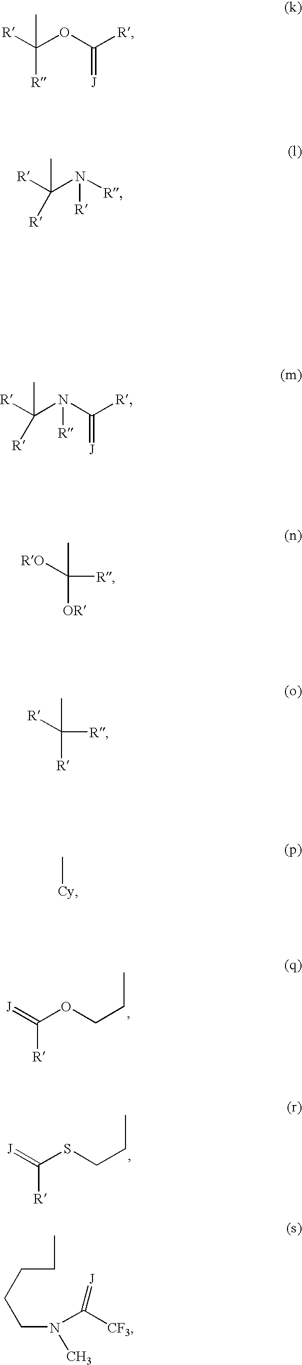 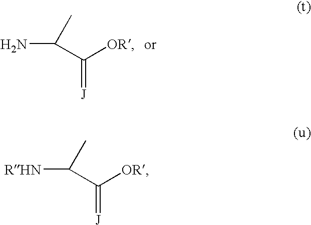
wherein:

each R′ and R″ independently is H; C₁₋₁₀ cyclic or acyclic, branched or unbranched alkyl, alkenyl, alkynyl; halo; O-alkyl; NH₂; NHMe; N(Me)₂; CN; acyl; aryl; heteroaryl; heterocycle; carbocycle; amino acid residue; or together with the atoms to which they are attached may form a 3-7 membered carbocyclic or heterocyclic ring;

J is O, S, or NR′″;

Cy is any optionally substituted carbocycle, heterocycle or heteroaryl;

R′″ is H, OH, SH, halo, optionally substituted C₁₋₄ alkyl, optionally substituted C₂₋₄ alkenyl or C₂₋₄ alkynyl, N₃, CN, CH₂CN, CH₂N₃, CH₂NH₂, CH₂NHCH₃, CH₂N(CH₃)₂, CH₂OH, halogenated alkyl, alkoxy, CF₃, C(A′)₃, 2-Br-ethyl, CH₂F, CH₂Cl, CH₂CF₃, CF₂CF₃, CH₂(A′), C(A′)₂(A′)₃, haloalkenyl, Br-vinyl, haloalkynyl; —(CH₂)ₘC(O)OR⁴, —O(acyl), —O(lower acyl), —O(alkyl), —O(lower alkyl), —O(alkenyl), CF₃, halogen, —NO₂, —NH₂, —(CH₂)ₘNHR⁴, —(CH₂)ₘN(R⁴)₂, —(CH₂)ₘC(O)NHR⁴, —(CH₂)ₘC(O)N(R⁴)₂, or C₃₋₇ cycloalkylamino, and where the optional substitutions on alkyl, alkenyl and/or alkynyl may be one or more halogen, hydroxy, amino, alkoxy, or alkylthio groups or atoms taken in any combination.

Alternatively, a purine base analogue having a reactive substituent at position 8 of a 2-amino-imidazo[1,2-a]-s-triazin-4-one, such as, for example, an ester substituent, may be reacted with ammonia and sodium hydroxide with appropriate pH adjustments to provide carboxylic acid and carboxamide substituents, or with ammonia and methanol to provide an alcohol substituent. The following non-limiting examples illustrate some general and specific methodologies to obtain the embodiments of the present invention.

A. General Condensation Reactions

### a. Synthesis of 2-Aminoimidazo[1,2-a]-s-triazin-4-one Derivative Compounds from 2-aminoimidazo[1,2-a]-s-triazin-4-one

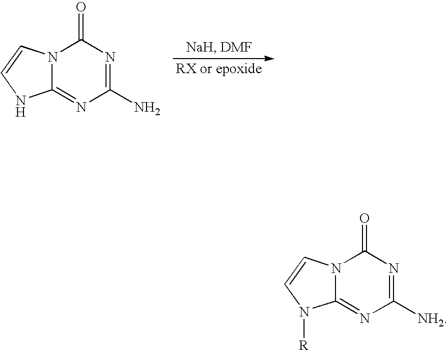

### b. Synthesis of 2-Amino-8-β-D-erythrofuranosylimidazo[1,2-a]-s-triazin-4-one from D-erythrose

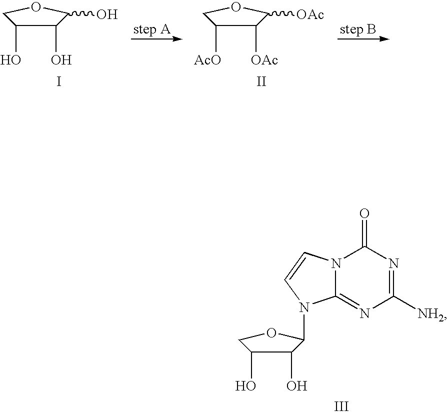 wherein pyridine and acetic anhydride are employed in step A, and hexamethyldisilazane with ammonium sulfate catalyst and acetonitrile are used in step B to produce the final product.

B. General Position-8 Reactive Group Syntheses

### a. Synthesis of 2-Aminoimidazo[1,2-a]-s-triazin-4-one Derivatives Compounds from 2-Amino-8-(ethyl-3-propionate)imidazo[1,2-a]-s-triazin-4-one (I)

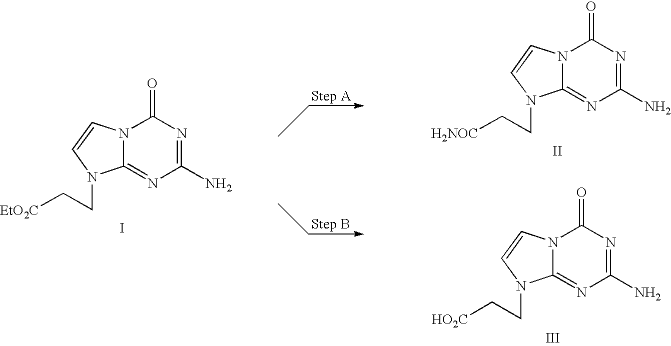

### b. Synthesis of 7-Chloro-1-(methylpivalate)imidazo[1,2-α]pyrimidin-5-one from imidazo[1,2-a]pyrimidin-5-one

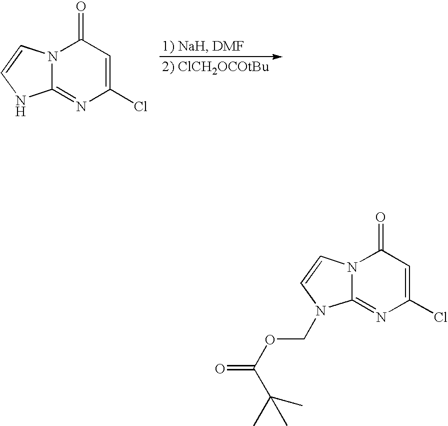 Nucleoside-Like Analogues

In particular, the synthesis of the nucleosides can be achieved by either alkylating the appropriately modified sugar, followed by glycosylation or glycosylation followed by alkylation of the nucleoside, though typically alkylating the appropriately modified sugar, followed by glycosylation. The following non-limiting embodiments illustrate some general and specific methodologies to obtain the nucleosides of the present invention.

A. General Synthesis of 1′-C-Branched Nucleosides

A 1′-C branched ribonucleoside of the following structure wherein R⁵ is the 1′-C branch substituent and an “R” substituent on any of Formulae (i), (ii), (iii), (iv), (v) or (vi) given above is depicted as: 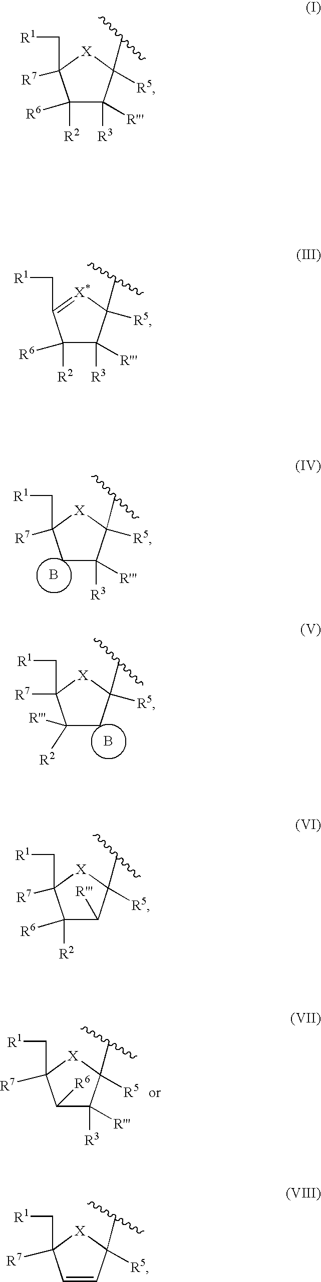
wherein:

each R¹, R², R³, R⁴, R⁵, R⁶, R⁷, X, X*, [symbol] A′, m, and R′″ is as defined above, or

R′″ and R³, together with the carbon atom to which they are attached, form an optionally substituted 3- to 7-membered saturated or unsaturated ring that optionally may have one or more heteroatoms selected from the group consisting of O, S, N or P;

except that R⁵ is OH, NH₂, or SH only when X or X* is C in Formulae I, III-VIII; and

all tautomeric, enantiomeric and stereoisomeric forms thereof, can be prepared by one of the following general methods.

Modification from the Lactone

The key starting material for this process is an appropriately substituted lactone. The lactone may be purchased or can be prepared by any known means including standard epimerization, substitution and cyclization techniques. The lactone optionally can be protected with a suitable protecting group, typically with an acyl or silyl group, by methods well known to those skilled in the art, as taught by Greene et al., Protective Groups in Organic Synthesis, John Wiley and Sons, Second Edition, 1991. The protected lactone can then be coupled with a suitable coupling agent, such as an organometallic carbon nucleophile like a Grignard reagent, an organolithium, lithium dialkylcopper or R⁶-SiMe₃ in TAF with the appropriate non-protic solvent at a suitable temperature, to give the 1′-alkylated sugar.

The optionally activated sugar can then be coupled to the base by methods well known to those skilled in the art, as taught by Townsend, Chemistry of Nuceleotides, Plenum Press, 1994. For example, an acylated sugar can be coupled to a silylated base with a Lewis acid such as tin tetrachloride, titanium tetrachloride, or trimethylsilyltriflate in the appropriate solvent at a suitable temperature.

Subsequently, the nucleoside can be deprotected by methods well known to those skilled in the art, as taught by Greene et al., Protective Groups in Organic Synthesis, John Wiley and Sons, Second Edition, 1991.

In a particular embodiment, the 1′-C-branched ribonucleoside is desired. Alternatively, dexoyribonucleoside is desired. To obtain these nucleosides, the formed ribonucleoside an optionally be protected by methods well known to those skilled in the art, as taught by Greene et al., Protective Groups in Organic Synthesis, John Wiley and Sons, Second Edition, 1991, and then the 2′-OH can be reduced with a suitable reducing agent. Optionally, the 2′-OH can be activated to facilitate reduction as, for example, via the Barton reduction.

Altenative syntheses for preparing 1′-C-branched nucleosides may be found in PCT Publication Serial No. WO 01/92282 and WO 01/90121, both by Idenix Pharmaceuticals.

B. General Synthesis of 2′-C-Branched Nucleosides

A 2′-C-branched ribonucleoside of the following structure wherein R′″ is the 2′-C branch substituent and an “R” substituent on any of Formulae (i), (ii), (iii), (iv), (v) or (vi) given above is depicted as one of the following structures, for example: 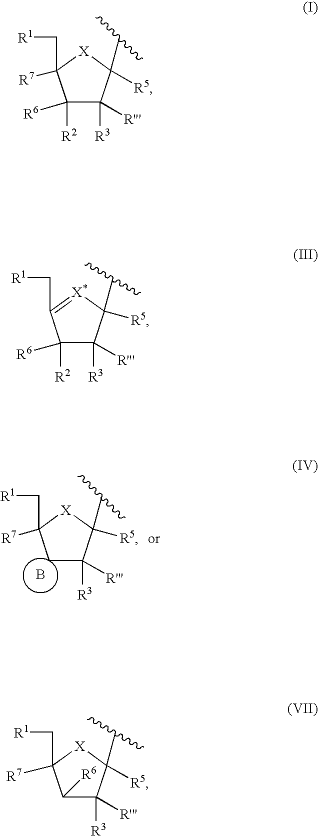
wherein:

each R¹, R², R³, R⁵, R⁶, R⁷, X, X*, [symbol] m, and R′″ is as defined above, or

R′″ and R³, together with the carbon atom to which they are attached, form an optionally substituted 3- to 7-membered saturated or unsaturated ring that optionally may have one or more heteroatoms selected from the group consisting of O, S, N or P;

except that R⁵ is OH, NH₂, or SH only when X or X* is C in Formulae I, III-VIII; and

all tautomeric, enantiomeric and stereoisomeric forms thereof, can be prepared by one of the following general methods.

Glycosylation of the Nucleoase with an Appropriately Modified Sugar

The key starting material for this process is an appropriately substituted sugar with a 2′-OH and 2′-H, with an appropriate leaving group (LG), such as an acyl or halogen group, for example. The sugar can be purchased or can be prepared by any known means including standard epimerization, substitution, oxidation and/or reduction techniques. The substituted sugar can then be oxidized with an appropriate oxidizing agent in a compatible solvent at a suitable temperature to yield the 2′-modified sugar. Possible oxidizing agents are Jones' reagent (a mixture of chromic and sulfuric acids), Collins' reagent (dipyridine Cr(VI)oxide), Corey's reagent (pyridinium chlorochromate), pyridinium dichromate, acid dichromate, potassium permanganate, MnO₂, ruthenium tetroxide, phase transfer catalysts such as chromic acid or permanganate supported on a polymer, Cl₂-pyridine, H₂O₂-ammonium molydate, NarO₂-CAN, NaOCl in HOAc, copper chromate, copper oxide, Raney nickel, palladium acetate, Meerwin-Pondorf-Verley reagent (aluminum t-utoxide with another ketone) and N-bromosuccinimide.

Then coupling of an organometallic carbon nucleophile such as a Grignard reagent, an organolithium, lithium dialkylcopper or R⁶-SiMe₃ in TAF with the ketone and an appropriate non-protic solvent at a suitable temperature, yields the 2′-alkylated sugar. The alkylated sugar optionally can be protected with a suitable protecting group, typically with an acyl or silyl group, by methods well known to those skilled in the art, as taught by Greene et al., Protective Groups in Organic Synthesis, John Wiley and Sons, Second Edition, 1991.

The optionally protected sugar can then be coupled to the base by methods well known to those skilled in the art, as taught by Townsend, Chemistry of Nucleosides and Nucleotides, Plenum Press, 1994. For example, an acylated sugar can be coupled to a silylated base with a Lewis acid, such as tin tetrachloride, titanium tetrachloride, or trimethylsilyltriflate in an appropriate solvent at a suitable temperature. Alternatively, a halo-sugar can e coupled to a silylated base in the presence of trimethylsilyltriflate.

Subsequently, the nucleoside can be deprotected by methods well known to those skilled in the art, as by Greene et al., Protective Groups in Organic Synthesis, John Wiley and Sons, Second Edition, 1991.

Other syntheses for preparing 2′-C-branched nucleosides may be found in PCT Publication Serial No. WO 01/92282 and WO 01/90121, both by Idenix Pharmaceuticals.

C. General Synthesis of 3′-C-Branched Nucleosides

A 3′-C-branched ribonucleoside of the following structure wherein R⁶ in Formulae (I), (III), and (VI), and R′″ in Formula (V) is the 3′-C branch substituent, and an “R” substituent on any of Formulae (i), (ii), (iii), (iv), (v) or (vi) given above is depicted as one of the following structures, for example: 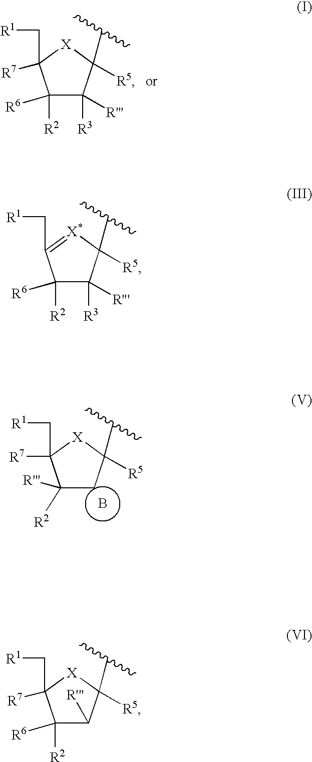
wherein:

each R¹, R², R³, R⁵, R⁶, R⁷, [symbol] and R“′ is as defined above;

X and X* are defined as above;

except that R⁵ is OH, NH₂, or SH only when X or X* is C in Formulae I, III, V, and VI; and

all tautomeric, enantiomeric and stereoisomeric forms thereof, can be prepared by one of the following general methods.

Glycosylation of the Nucleoase with an Appropriately Modified Sugar.

The key starting material for this process is an appropriately substituted sugar with a 3′-OH and a 3′-H, with an appropriate leaving group (LG) such as, for example, an acyl group or a halogen. The sugar can be purchased or can be prepared by any known means including standard epimerization, substitution, oxidation and/or reduction techniques. The substituted sugar then can be oxidized by an appropriate oxidizing agent in a compatible solvent at a suitable temperature to yield the 3′-modified sugar.

Possible oxidizing agents include Jones' reagent (a mixture of chromic and sulfuric acids), Collins' reagent (dipyridine Cr(VI)oxide), Corey's reagent (pyridinium chlorochromate), pyridinium dichromate, acid dichromate, potassium permanganate, MnO₂, ruthenium tetroxide, phase transfer catalysts such as chromic acid or permanganate supported on a polymer, Cl₂-pyridine, H₂O₂-ammonium molydate, NarO₂—CAN, NaOCl in HOAc, copper chromate, copper oxide, Raney nickel, palladium acetate, Meerwin-Pondorf-Verley reagent (aluminum t-utoxide with another ketone) and N-bromosuccinimide.

Then coupling of an organometallic carbon nucleophile such as a Grignard reagent, an organolithium, lithium dialkylcopper or R⁶-SiMe₃ in TAF with the ketone and an appropriate non-protic solvent at a suitable temperature, yields the 3′-C-branched sugar. The 3′-C-branched sugar optionally can e protected with a suitable protecting group, typically with an acyl or silyl group, by methods well known to those skilled in the art, as taught by Greene et al., Protective Groups in Organic Synthesis, John Wiley and Sons, Second Edition, 1991.

The optionally protected sugar can then be coupled to the base by methods well known to those skilled in the art, as taught y Townsend, Chemistry of Nucleosides and Nucleotides, Plenum Press, 1994. For example, an acylated sugar can e coupled to a silylated base with a Lewis acid, such as tin tetrachloride, titanium tetrachloride, or trimethylsilyltriflate in an appropriate solvent at a suitable temperature. Alternatively, a halo-sugar can be coupled to a silylated base in the presence of trimethylsilyltriflate.

Subsequently, the nucleoside can be deprotected by methods well known to those skilled in the art, as by Greene et al., Protective Groups in Organic Synthesis John Wiley and Sons, Second Edition, 1991.

In a particular embodiment, the 3′-C-branched ribonucleoside is desired. Alternatively, a deoxyribonucleoside is desired. To obtain these nucleosides, the formed ribonucleoside can optionally be protected by methods well known to those skilled in the art, as by Greene et al., Protective Groups in Organic Synthesis, John Wiley and Sons, Second Edition, 1991, and then the 2′-OH can be reduced with a suitable reducing agent. Optionally, the 2′-OH can be activated to facilitate reduction, such as, for example, by the Barton reduction.

Alternative syntheses for preparing 3′-C-branched nucleosides may be found in PCT Publication Serial No. WO 01/92282 and WO 01/90121, both by Idenix Pharmaceuticals.

D. General Synthesis of 4′-C-Branched Nucleosides

A 4′-C branched ribonucleoside of the following structure wherein R⁷ is the 4′-C branch substituent, and an “R” substituent on any of Formulae (i), (ii), (iii), (iv), (v) or (vi) is given above is depicted as: 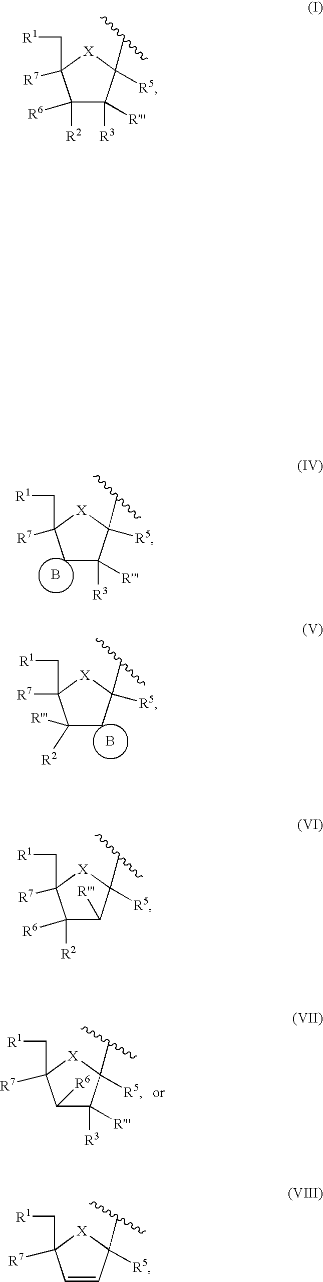
wherein:

each R¹, R², R³, R⁵, R⁶, R⁷, X, X*, [symbol] and R′″ is defined as above;

except that R⁵ is OH, NH₂, or SH only when X or X* is C in Formulae I, IV, V, VI, VII and VIII; and

all tautomeric, enantiomeric and stereoisomeric forms thereof, can be prepared by one of the following general methods.

Modification from the Pentodialdo-Furanose.

The key starting material for this process is an appropriately substituted pentodialdo-furanose. The pentodialdo-furanose can be purchased or can be prepared by any known means including standard epimerization, sustitution and cyclization techniques.

In one embodiment, the pentodialdo-furanose is prepared from the appropriately substituted hexose. The hexose can be purchased or can be prepared by any known means including standard epimerization (for example, via alkaline treatment), sustitution, and coupling techniques. The hexose can be in either the furanose form or cyclized by any means known in the art, such as methodology taught by Townsend in Chemistry of Nucleosides and Nucleotides, Plenum Press, 1994, typically by selectively protecting the hexose, to give the appropriate hexafuranose.

The 4′-hydroxymethylene of the hexafuranose then can be oxidized with an appropriate oxidizing agent in a compatible solvent at a suitable temperature to yield the 4′-aldo-modified sugar. Possible oxidizing agents are Swern reagents, Jones' reagent (a mixture of chromic and sulfuric acids), Collins' reagent (dipyridine Cr(VI)oxide), Corey's reagent (pyridinium chlorochromate), pyridinium dichromate, acid dichromate, potassium permanganate, MnO₂, ruthenium tetroxide, phase transfer catalysts such as chromic acid or permanganate supported on a polymer, Cl₂-pyridine, H₂O₂-ammonium molydate, NarO₂-CAN, NaOCl in HOAc, copper chromate, copper oxide, Raney nickel, palladium acetate, Meerwin-Pondorf-Verley reagent (aluminum t-utoxide with another ketone) and N-bromosuccinimide, although using H₃PO₄, DMSO and DCC in a mixture of benzene/pyridine at room temperature is common.

Then the pentodialdo-furanose optionally can be protected with a suitable protecting group, typically with an acyl or silyl group, by methods well known to those skilled in the art, as taught by Greene et al., Protective Groups in Organic Synthesis, John Wiley and Sons, Second Edition, 1991. In the presence of a base, such as sodium hydroxide, the protected pentodialdo-furanose then can be coupled with a suitable electrophilic alkyl, halogeno-alkyl (such as CF₃), alkenyl or alkynyl (i.e., allyl), to obtain the 4′-alkylated sugar. Alternatively, the protected pentodialdo-furanose can be coupled with a corresponding carbonyl, such as formaldehyde, in the presence of a base like sodium hydroxide and with an appropriate polar solvent like dioxane, at a suitable temperature, and then reduced with an appropriate reducing agent to provide the 4′-alkylated sugar. In one embodiment, the reduction is carried out using PhOC(S)Cl and DMAP in acetonitrile at room temperature, followed by reflux treatment with ACCN and TMSS in toluene.

The optionally activated sugar can be coupled to the base by methods well known to those skilled in the art, as taught by Townsend in Chemistry of Nucleosides and Nucleotides, Plenum Press, 1994. For example, an acylated sugar can be coupled to a silylated base with a Lewis acid, such as tin tetrachloride, titanium tetrachloride, or trimethylsilyltriflate in an appropriate solvent at room temperature.

Subsequently, the nucleoside can be deprotected by methods well known to those skilled in the art, as by Greene et al., Protective Groups in Organic Synthesis, John Wiley and Sons, Second Edition, 1991.

In a particular embodiment, the 4′-C-branched ribonucleoside is desired; in another embodiment, a deoxyribonucleoside is desired. To obtain these nucleosides, the formed ribonucleoside can optionally be protected by methods well known to those skilled in the art, as by Greene et al., Protective Groups in Organic Synthesis, John Wiley and Sons, Second Edition, 1991, and then the 2′-OH can be reduced with a suitable reducing agent. Optionally, the 2′-OH can be activated to facilitate reduction, such as, for example, by the Barton reduction.

In yet another embodiment of the invention, the L-enantiomers are desired. These L-enantiomers corresponding to the compounds of the invention may be prepared following the same general methods given above, but beginning with the corresponding L-sugar or nucleoside L-enantiomer as the starting material.

Alternative syntheses for preparing 4′-C-branched nucleosides may be found in PCT Publication Serial No. WO 01/92282 and WO 01/90121, both by Idenix Pharmaceuticals.

E. Alternative Methods for Ribofuranosyl-5-aza-7-Deazapurine Synthesis

### Preparation of 1′-C-methyl-ribofuranosyl-5-aza-7-deazapurine via 6-amino-9-(1-deoxy-beta-D-psicofuranosyl)purine

As an alternative method of preparation, the title compound can be prepared according to the pulished procedure of Farkas and Sorm (J. Farkas and F. Sorm, “Nucleic acid components and their analogues. XCIV. Synthesis of 6-amino-9-(1-deoxy-beta-D-psicofuranosyl)purine,” Collect. Czech. Chem. Commun., 1967, 32:2663-7; and J. Farkas, Collect. Czech. Chem. Commun., 1966, 31:1535 (Scheme 7).

In a similar manner, but using the appropriate sugar and 5-aza-7-deazapurine base corresponding to the desired product compound, a variety of Formula (I) compounds can be prepared.

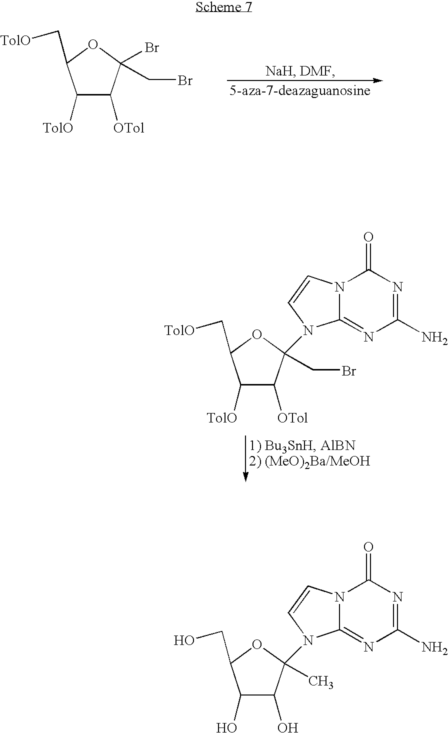

### Preparation of ribofuranosyl-5-aza-7-deazapurines via use of Protective Groups

As an alternative method of preparation, the compounds of the present invention can also be prepared by synthetic methods well known to those skilled in the art of nucleoside and nucleotide chemistry, such as taught by Townsend in Chemistry of Nucleosides and Nucleotides, Plenum Press, 1994.

A representative general synthetic method is provided in Scheme 8. The starting material is a 3,5-is-O-protected beta-D-alkyl ribofuranoside, but it will be understood that any 2′, 3′, or 5′-position may carry a protecting group to shield it from reacting. The 2′-C—OH then is oxidized with a suitable oxidizing agent in a compatible solvent at a suitable temperature to yield the 2′-keto-modified sugar. Possible oxidizing agents are Swern reagents, Jones' reagent (a mixture of chromic and sulfuric acids), Collins' reagent (dipyridine Cr(VI)oxide), Corey's reagent (pyridinium chlorochromate), pyridinium dichromate, acid dichromate, potassium permanganate, MnO₂, ruthenium tetroxide, phase transfer catalysts such as chromic acid or permanganate supported on a polymer, Cl₂-pyridine, H₂O₂-ammonium molydate, NarO₂-CAN, NaOCl in HOAc, copper chromate, copper oxide, Raney nickel, palladium acetate, Meerwin-Pondorf-Verley reagent (aluminum t-utoxide with another ketone) and N-bromosuccinimide.

Next, addition of a Grignard reagent, such as, for example, an alkyl-, alkenyl- or alkynyl-magnesium halide like CH₃MgBr, CH₃CH₂MgBr, vinylMgBr, allylMgBr and ethynyhMgBr, or an alkyl-, alkenyl- or alkynyl-lithium, such as CH₃Li, in a suitable organic solvent, such as, for example, diethyl ether or THF, across the double bond of the 2′-carbonyl group provides a tertiary alcohol at this position. The addition of a hydrogen halide in a suitable solvent, such as, for example, HBr in HOAc, in the subsequent step provides a leaving group (LG) such as, for example, a chloro, bromo or iodo, at the C-1 anomeric carbon of the sugar ring that later generates a nucleosidic linkage. Other suitable LGs include C-1 sulfonates such as, for example, methanesulfonate, trifluoromethanesulfonate and/orp-toluenesulfonate.

The introduction in the next step of a metal salt (Li, Na or K) of an appropriately substituted 2-azapurine in a suitable organic solvent such as, for example, THF, acetonitrile of DMF, results in the formation of the desired nucleosidic linkage and addition of the desired 2-azapurine base. This displacement reaction may be catalyzed by a phase transfer catalyst like TDA-1 or triethylbenzylammonium chloride. The introduction of a “Z” sustituent on any of base formulae (i)-(vi) optionally may be performed subsequent to the initial addition of protecting groups. For example, the introduction of an amino group for “Z” is accomplished by the addition of an appropriate amine in an appropriate solvent to the 2′-C-halo intermediate just prior to the last step of removal of the protecting groups. Appropriate amines include alcoholic or liquid ammonia to generate a primary amine (—NH₂), an alkylamine to generate a secondary amine (—NHR), or a dialkylamine to generate a tertiary amine (—NRR′).

Finally, the nucleoside can be deprotected by methods well known to those skilled in the art, as by Greene et al., Protective Groups in Organic Synthesis, John Wiley and Sons, Second Edition, 1991.

The present invention is described by way of illustration in the following examples. It will be understood by one of ordinary skill in the art that these examples are in no way limiting and that variations of detail can be made without departing from the spirit and scope of the present invention.

### EXAMPLES

The following abbreviations for certain reagents used in the working examples and their definitions are: DCM is dichloromethane; DCE is dichloroethane; DMF is dimethylformamide; TFA is trifluoroacetyl; TMSCl is trimethylsilyl chloride; TsCl is tosyl chloride; and TFA is trifluoroacetyl.

### Example 1

This scheme illustrates the synthesis of 2-Aminoimidazo[1,2-a]-s-triazin-4-one derivative compounds from 2-Aminoimidazo[1,2-a]-s-triazin-4-one:

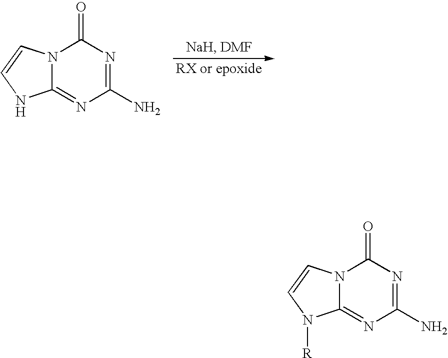

The typical procedure for the preparation of 2-Aminoimidazo[1,2-a]-s-triazin-4-one derivatives is:

To a suspension of sodium hydride (60% in oil, 1.2 eq.) in dry dimethylformamide (0.2M) was added 2-Aminoimidazo[1,2-a]-s-triazin-4-one [for preparation see Journal of Medicinal Chemistry, 1978, Vol 21, No 9, 8831 (1 eq.) at 20° C. and stirred for one hour. Alkyl halide or epoxide (1.1 eq.) was added and the solution was allowed to react for several hours (see the following table 1). After the end of the reaction, the mixture was evaporated to dryness. The residue was purified on silica gel or reverse-phase column to give the title compound. R is as defined above in the specification.

The following Table 1 is based upon the synthesis provided above.

TABLE 1

| reagents | experiments | products | yields |
|---|---|---|---|
| CH₃CH₂Br | 20° C., 20 h | 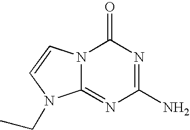 | 69% |
| 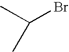 | reflux, 20 h | 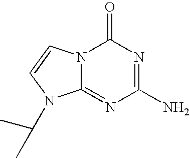 | 33% |
| BrCH₂CH₂Br | 40° C., 2 days | 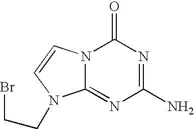 | 23% |
| 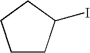 | 30° C., 17 h 80° C., 1.5 h | 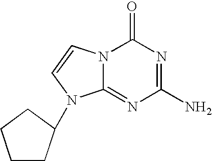 | 19% |
| 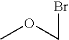 | 40° C., 24 h | 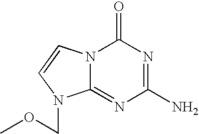 | 32% |
| 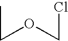 | 100° C., 24 h | 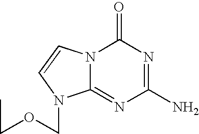 | 20% |
| HOCH₂CH₂I | 40° C., 2 days | 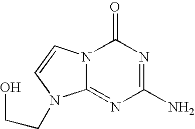 | 40% |
| 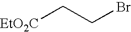 | 130° C., 3 days, +NaI | 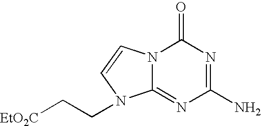 | 40% |
| 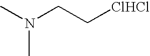 | 50° C., 24 h | 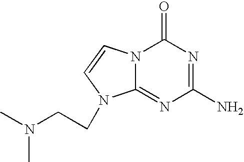 | 48% |
| 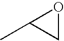 | 120° C., 2 days | 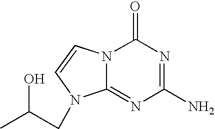 | 41% |
| 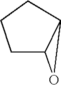 | 120° C., 2 days | 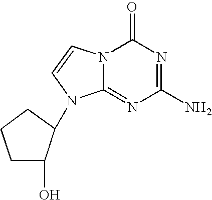 | 55% |

The following physical data relate in order to the products contained in Table 1:

### Example 1.1

### 2-Amino-8-ethylimidazo[1,2-a]-s-triazin-4-one

¹H NMR (DMSO-d₆) δ ppm: 1.30 (t, 2H, J=7.2 Hz, CH₃), 3.9 (q, 2H, J=7.2 Hz, CH₂), 6.85 (br, 2H, NH₂), 7.35 (m, 2H, CH)

Mass spectrum: m/z (FAB>0) 359 (2M+H)⁺, 180 (M+H)⁺

### Example 1.2

### 2-Amino-8-isopropylimidazo[1,2-a]-s-triazin-4-one

¹H NMR (DMSO-d₆) δ ppm: 1.40 (m, 6H, CH₃), 4.55 (m, 1H, CH), 6.85 (br, 2H, NH₂), 7.35 (d, 1H, J=2.6 Hz, CH), 7.42 (d, 1H, J=2.6 Hz, CH)

Mass spectrum: m/z (FAB>0) 387 (2M+H)⁺, 194 (M+H)⁺, (FAB<0) 192 (M-H)⁻

### Example 1.3

### 2-Amino-8-(bromoethyl)imidazo[1,2-a]-s-triazin-4-one

¹H NMR (DMSO-d₆) δ ppm: 3.88 (t, 2H, J=6.2 Hz, CH₂), 4.30 (t, 2H, J=6.2 Hz, CH₂), 6.92 (br, 2H, NH₂), 7.32 (d, 1H, J=2.6 Hz, CH), 7.35 (d, 1H, J=2.6 Hz, CH)

### Example 1.4

### 2-Amino-8-cyclopentylimidazo[1,2-a]-s-triazin-4-one

¹H NMR (DMSO-d₆) δ ppm: 1.6-2.13 (m, 8H), 4.65 (m, 1H, CH), 6.85 (br, 2H, NH₂), 7.35 (d, 1H, J=2.7 Hz, CH), 7.39 (d, 1H, J=2.7 Hz, CH)

Mass spectrum: m/z (FAB>0) 439 (2M+H)⁺, 220 (M+H)⁺

### Example 1.5

### 2-Amino-8-(methoxymethyl)imidazo[1,2-a]-s-triazin-4-one

¹H NMR (DMSO-d₆) δ ppm: 3.4 (s, 3H, CH₃), 5.34 (s, 2H, CH₂), 7.12 (br, 2H, NH₂), 7.32 (d, 1H, J=2.7 Hz, CH), 7.35 (d, 1H, J=2.7 Hz, CH)

Mass spectrum: m/z (FAB>0) 391 (2M+H)⁺, 196 (M+H)⁺

### Example 1.6

### 2-Amino-8-(ethoxymethyl)imidazo[1,2-a]-s-triazin-4-one

¹H NMR (DMSO-d₆) δ ppm: 1.08 (t, 2H, J=7.0 Hz, CH₃), 3.52 (q, 2H, J=7.0 Hz, CH₂), 5.30 (s, 2H, CH₂), 6.98 (br, 2H, NH₂), 7.35 (d, 1H, J=2.6 Hz, CH), 7.37 (d, 1H, J=2.6 Hz, CH)

Mass spectrum: m/z (FAB>0) 419 (2M+H)⁺, 210 (M+H)⁺

### Example 1.7

### 2-Amino-8-(2-hydroxyethyl)imidazo[1,2-a]-s-triazin-4-one

¹H NMR (DMSO-d₆) δ ppm: 3.6 (m, 2H, CH₂), 3.94 (m, 2H, CH₂), 4.96 (m, 1H, OH), 6.84 (br, 2H, NH₂), 7.24 (d, 1H, J=2.6 Hz, CH), 7.43 (d, 1H, J=2.6 Hz, CH)

Mass spectrum: m/z (FAB>0) 391 (2M+H)⁺, 196 (M+H)⁺

### Example 1.8

### 2-Amino-8-(ethyl-3-propionate)imidazo[1,2-a]-s-triazin-4-one

¹H NMR (DMSO-d₆) δ ppm: 1.10 (t, 3H, J=7.1 Hz, CH₃), 2.88 (t, 2H, J=6.8 Hz, CH₂), 4.07 (q, 2H, J=7.1 Hz, CH₂), 4.16 (t, 3H, J=6.8 Hz, CH₂), 7.20 (br, 1H, NH), 7.42 (d, 1H, J=2.6, CH), 7.54 (d, 1H, J=2.6, CH), 7.95 (br, 1H, NH)

Mass spectrum: m/z (FAB>0) 503 (2M+H)⁺, 252 (M+H)⁺

### Example 1.9

### 2-Amino-8-(2-dimethylaminoethyl)imidazo[1,2-a]-s-triazin-4-one

¹H NMR (DMSO-d₆) δ ppm: 2.18 (s, 6H, CH₃), 2.58 (t, 2H, J=6.2 Hz, CH₂), 3.95 (t, 2H, J=6.2 Hz, CH₂), 6.85 (br, 2H, NH₂), 7.3 (m, 2H, CH)

Mass spectrum: m/z (FAB>0) 445 (2M+H)⁺, 223 (M+H)⁺, (FAB<0) 221 (M-H)⁻

### Example 1.10

### 2-Amino-8-(2-hydroxypropyl)imidazo[1,2-a]-s-triazin-4-one

¹H NMR (DMSO-d₆) δ ppm: 1.07 (d, 3H, J=6.2 Hz, CH₃), 3.67-3.88 (m, 2H, CH₂), 3.98 (m, 1H), 4.97 (m, 1H, OH), 6.81 (br, 2H, NH₂), 7.21 (d, 1H, J=2.6 Hz, CH), 7.28 (s, 1H, J=2.6 Hz, CH)

Mass spectrum: m/z (FAB>0) 419 (2M+H)⁺, 210 (M+H)⁺

### Example 1.11

### 2-Amino-8-(trans-hydroxycyclopentan-2-ol)imidazo[1,2-a]-s-triazin-4-one

¹H NMR (DMSO-d₆) δ ppm: 1.47-2.18 (m, 6H), 4.2-4.42 (m, 2H), 5.18 (d, 1H, J=4.6 Hz, OH), 6.86 (br, 2H, NH₂), 7.37 (d, 1H, J=2.7 Hz, CH), 7.43 (d, 1H, J=2.7 Hz, CH)

Mass spectrum: m/z (FAB>0) 471(2M+H)⁺, 236 (M+H)⁺, (FAB<0) 234 (M-H)^{-.}

### Example 2

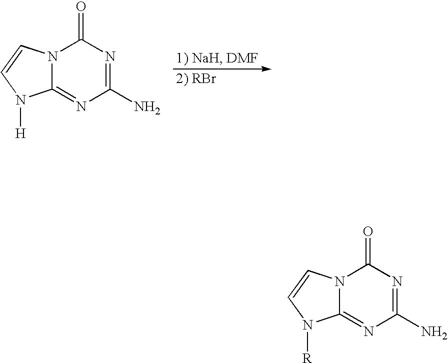

To a suspension of 2-aminoimidazo[1,2-a]-s-triazin-4-one (600 mg) in dry dimethylformamide (20 mL) was added sodium hydride (60% in oil, 168 mg, 4.2 mmoles) at 20° C. and stirred for one hour. Bromide derivative (1.3 eq) was added and stirred at 20° C. for 16 hours. The reaction mixture was evaporated to dryness. The residue was purified on silica gel using dichloromethane/methanol as eluant to give the title compound as a white powder.

The following compounds were prepared according to this process:

| Identifier | R | Nomenclature |
|---|---|---|
| A | C₆H₅CH₂ | 2-Amino-N8-benzyl- |
| | | imidazo[1,2-a]-s- |
| | | triazin-4-one |
| B | C₅H₁₁ | 2-Amino-8-n- |
| | | pentylimidazo[1,2-a]]-s- |
| | | triazin-4-one |
| C | C₆H₅CH═CHCH₂ | 1-Cinnamyl-(2- |
| | | Aminoimidazo[1,2-a]-s- |
| | | triazin-4-one) |
| D | C₆H₁₃ | 2-Amino-8-n-hexyl- |
| | | imidazo[1,2-a]-s- |
| | | triazin-4-one |
| E | C₂H₄O₂CH(CH₂) | 2-Amino-8-(1,3-dioxolane-2- |
| | | ethyl)imidazo[1,2-a]-s- |
| | | triazin-4-one |
| F | CH₂═CHCH₂ | 2-Amino-8-allyl- |
| | | imidazo[1,2-a]-s- |
| | | triazin-4-one |
| G | C₂H₅OCO(CH₂)₃ | 2-Amino-8-(ethyl-1- |
| | | butyrate)imidazo[1,2-a]- |
| | | s-triazin-4-one |
| H | CH₃CO₂(CH₂)₄ | 2-Amino-8-(acetate-1- |
| | | butyloxy)imidazo[1,2- |
| | | a]-s-triazin-4-one |
| I | C₅H₅NCH₂ | 2-Amino-8-(2- |
| | | methylpyridine)imidazo[1,2- |
| | | a]-s-triazin-4-one |
| J | CN(CH₂)₃ | 2-Amino-8-(4- |
| | | butyronitrile)imidazo[1,2- |
| | | a]-s-triazin-4-one |
| K | CH₃CH₃(CH₂)₃ | 2-Amino-8-(4- |
| | | isopentyl)imidazo[1,2- |
| | | a]-s-triazin-4-one |

### Example 2.1

### 2-Amino-N8-benzyl-imidazo[1,2-a]-s-triazin-4-one

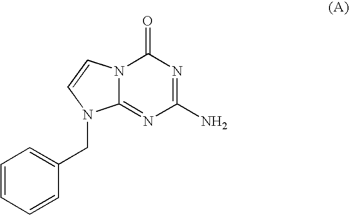

¹H NMR (DMSO-d₆) δ ppm: 5.12 (s, 2H, CH₂), 6.86 (s, 2H, NH₂), 7.35 (m, 7H, CH base and Ph)

Mass spectrum: m/z (FAB>0) 242 (M+H)⁺, 483 (2M+H)⁺

### Example 2.2

### 2-Amino-8-n-pentylimidazo[1,2-a]]-s-triazin-4-one

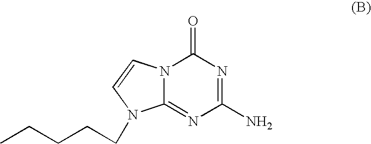

¹H NMR (DMSO-d₆) δ ppm: 0.85 (t, 3H, CH₃, J=6.8), 1.28 (m, 2H, CH₂), 1.71 (m, 2H, CH₂), 3.86 (t, 2H, CH₂, J=7.1 Hz), 6.86 (s, 2H, NH₂), 7.32 (syst AB, 2H, CH base)

Mass spectrum: m/z (FAB>0) 227 (M+H)⁺, 443 (2M+H)⁺

### Example 2.3

### 1-Cinnamyl-(2-Aminoimidazo[1,2-a]-s-triazin-4-one)

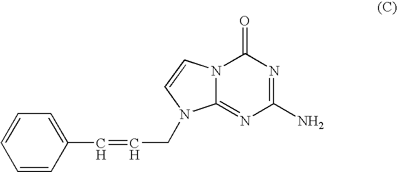

¹H NMR (DMSO-d₆) δ ppm: 4.70 (d, 2H, CH₂, J=4.8), 6.46 (m, 2H, CH₂), 6.93 (s, 2H, NH₂), 7.38 (3, 7H, CH base and Ph)

Mass spectrum: m/z (FAB>0) 268 (M+H)⁺

### Example 2.4

### 2-Amino-8-n-hexyl-imidazo[1,2-a]-s-triazin-4-one

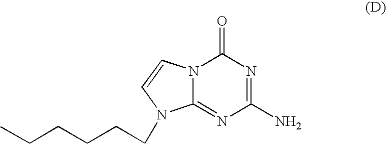

¹H NMR (DMSO-d₆) δ ppm: 0.70 (t, 3H, CH₃), 0.85 (m, 6H, CH₃), 1.71 (m, 2H, CH₂), 3.87 (t, 2H, CH₂, J=8.0 Hz), 6.88 (s, 2H, NH₂), 7.33 (syst AB, 2H, CH base)

Mass spectrum: m/z (FAB>0) 236 (M+H)⁺, 471 (2M+H)⁺

### Example 2.5

### 2-Amino-8-(1,3-dioxolane-2-ethyl)imidazo[1,2-a]-s-triazin-4-one

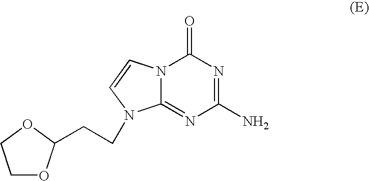

¹H NMR (DMSO-d₆) δ ppm: 2.08 (m, 2H, CH₂), 3.89 (m, 6H, CH₂), 4.87 (m, 1H, CH), 6.89 (s, 2H, NH₂), 7.32 (s, 2H, CH base)

Mass spectrum: m/z (FAB>0) 252 (M+H)⁺, 503 (2M+H)⁺

### Example 2.6

### 2-Amino-8-allyl-imidazo[1,2-a]-s-triazin-4-one

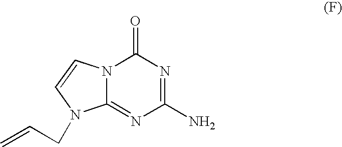

¹H NMR (DMSO-d₆) δ ppm: 4.62 (m, 2H, CH₂), 5.13 (dd, 1H, CH, J=26 Hz, J=1.3 Hz), 5.24 (dd, 1H, CH, J=26 Hz, J=1.3 Hz), 5.95 (m, 1H, CH), 6.89 (s, 2H, NH₂), 7.29 (syst AB, 2H, CH base)

Mass spectrum: m/z (FAB>0) 192 (M+H)⁺, 383 (2M+H)⁺

### Example 2.7

### 2-Amino-8-(ethyl-1-butyrate)imidazo[1,2-a]-s-triazin-4-one

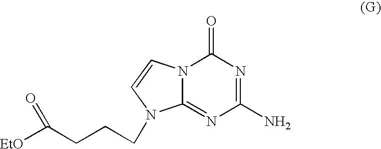

¹H NMR (DMSO-d₆) δ ppm: 1.06 (t, 3H, CH₃, J=7.0 Hz), 1.87 (m, 2H, CH₂), 2.02 (m, 2H, CH₂), 3.92 (t, 2H, CH₂, J=6.8 Hz), 4.07 (q, 1H, , CH₂, J=7.0 Hz, J=12.6 Hz), 6.85 (s, 2H, NH₂), 7.30 (syst AB, 2H, CH base)

Mass spectrum: m/z (FAB>0) 266 (M+H)⁺, 531 (2M+H)⁺

### Example 2.8

### 2-Amino-8-(acetate-1-butyloxy)imidazo[1,2-a]-s-triazin-4-one

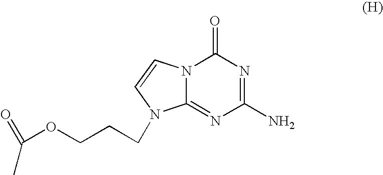

¹H NMR (DMSO-d₆) δ ppm: 1.60 (m, 2H, CH₂), 1.77 (m, 2H, CH₂), 2.02 (s, 3H, CH₃), 3.93 (t, 2H, CH₂, J=7.1 Hz), 4.04 (t, 2H, CH₂, J=6.4 Hz), 6.88 (s, 2H, NH₂), 7.35 (syst AB, 2H, CH base)

Mass spectrum: m/z (FAB>0) 266 (M+H)⁺, 531 (2M+H)⁺

### Example 2.9

### 2-Amino-8-(2-methylpyridine)imidazo[1,2-a]-s-triazin-4-one

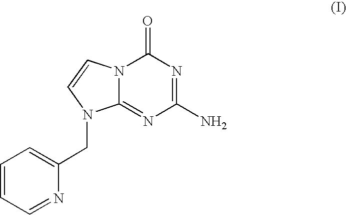

¹H NMR (DMSO-d₆) δ ppm: 5.25 (s, 2H, CH₂), 6.88 (s, 2H, NH₂), 7.37 (m, 4H, CH base CH pyr), 7.79 (dt, 1H, J=1.8 Hz, J=7.7 Hz), 8.53 (dd, 1H, J=0.8 Hz, J=1.8 Hz)

Mass spectrum: m/z (FAB>0) 244 (M+H)⁺, 485 (2M+H)⁺

### Example 2.10

### 2-Amino-8-(4-butyronitrile)imidazo[1,2-a]-s-triazin-4-one

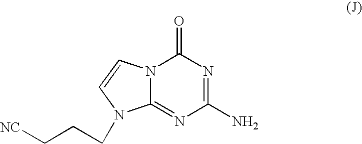

¹H NMR (DMSO-d₆) δ ppm: 2.06 (m, 2H, CH₂), 2.52 (m, 4H, NH₂), 3.96 (t, 2H, CH₂, J=6.9 Hz), 6.87 (s, 2H, NH₂), 7.33 (syst AB, CH base)

Mass spectrum: m/z (FAB>0) 219 (M+H)⁺, 437 (2M+H)⁺

### Example 2.11

### 2-Amino-8-(4-isopentyl)imidazo[1,2-a]-s-triazin-4-one

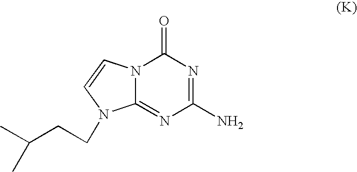

¹H NMR (DMSO-d₆) δ ppm: 0.85 (d, 6H, 2×CH₃, J=6.4 Hz), 1.47 (m, 3H, CH/CH₂) 3.90 (t, 2H, CH₂, J=7.0 Hz), 6.86 (s, 2H, NH₂), 7.33 (s, 2H, CH base)

Mass spectrum: m/z (FAB>0) 222 (M+H)⁺, 443 (2M+H)⁺

### Example 3

The following is a generalized, exemplary synthetic method for purine derivatives having a reactive group at the N-8 position of 2-Aminoimidazo[1,2-a]-s-triazin-4-one derivative compounds.

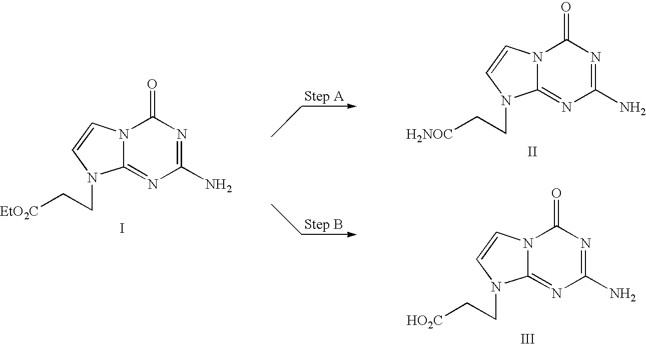

Step A: Compound I was suspended in an ammonia solution (28%). The mixture was stirred at 20° C. for 3 days. Then the pH of the solution was adjusted to pH 7-8 by the addition of 1N hydrochloric acid. A solid was deposited, collected, and washed with acetonitrile to give the title Compound II.

Step B: Compound I was suspended in 2M sodium hydroxide solution. The mixture was stirred at 20° C. for 3 hours. Next the pH of the solution was adjusted to pH 7-8 by addition of 1N hydrochloric acid. The reaction mixture was evaporated to dryness, and the residue was purified on a reverse-phase column using water as the eluant to provide the title compound, Compound III.

### Example 3.1

### 2-Amino-8-(1-butyloxy)imidazo[1,2-a]-s-triazin-4-one

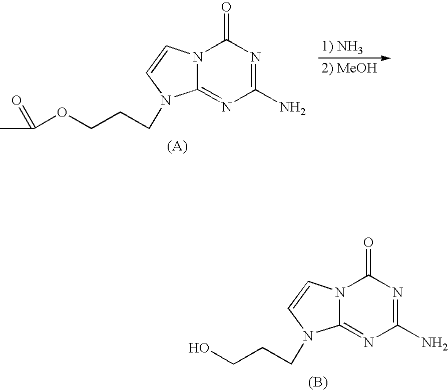

Compound A (460 mg) was suspended in a saturated amonical methanol solution (10 ml). The reaction mixture was stirred for 14 hours at room temperature. Solvents were removed under vacuo and the residue was purified on a silica gel column (DCM/EtOH 9/1) to afford the compound 2-Amino-8-(1-butyloxy)imidazo[1,2-a]-s-triazin-4-one, Compound B (189 mg).

¹H NMR (DMSO-d₆) δ ppm: 1.40 (m, 2H, CH₂), 1.78 (m, 2H, CH₂), 3.42 (q, 2H, CH₂, J=11.5 Hz, J=6.2 Hz), 3.92 (t, 2H, CH₂, J=7.1 Hz), 4.48 (t, 1H, OH, J=5.1 Hz), 6.87 (s, 2H, NH₂), 7.36 (syst AB, 2H, CH base)

Mass spectrum: m/z (FAB>0) 224 (M+H)⁺

### Example 3.2

### 2-Amino-8-(1-butylcarboxamide)imidazo[1,2-a]-s-triazin-4-one

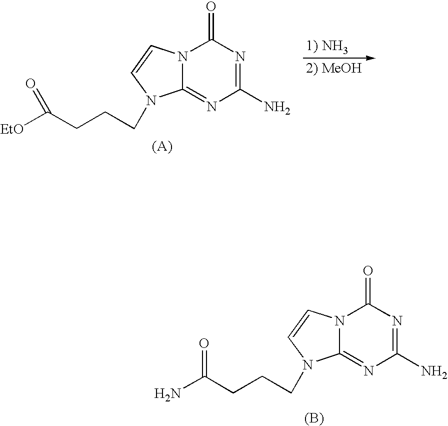

Compound (A) (296 mg) was suspended in a saturated amonical methanol solution (10 ml). The reaction mixture was stirred for 14 hours at room temperature. The precipitate was filtered off and washed with fresh methanol to afford the compound 2-Amino-8-(1-butylcarboxamide)imidazo[1,2-a]-s-triazin-4-one, Compound (B) (171 mg).

¹H NMR (DMSO-d₆) δ ppm: 1.97 (m, 2H, CH₂), 2.09 (q, 2H, CH₂), 3.92 (t, 2H, CH₂, J=6.9 Hz), 6.85 (s, 1H, NH₂), 6.89 (s, 3H, NH₂), 7.35 (syst AB, 2H, CH base)

Mass spectrum: m/z (FAB>0) 237 (M+H)⁺, 473 (2M+H)⁺; (FAB<0) 235 (M+H)⁻

### Example 3.3

This scheme illustrates the synthesis of 2-Aminoimidazo[1,2-a]-s-triazin-4-one derivatives compounds from 2-Amino-8-(ethyl-3-propionate)imidazo[1,2-a]-s-triazin-4-one (I):

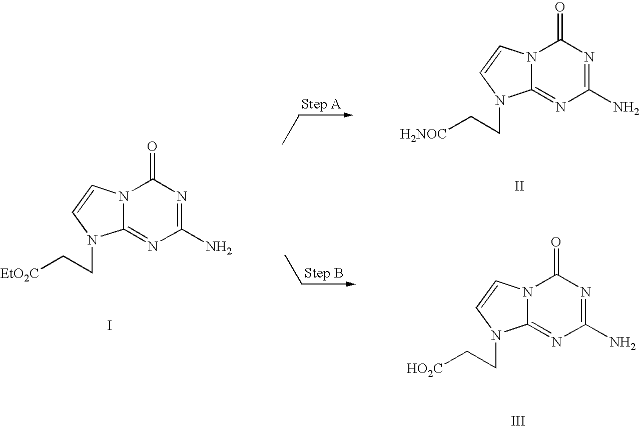

### Step A: 2-Amino-8-(3-propionamide)imidazo[1,2-a]-s-triazin-4-one

To a solution of 2-Amino-8-(ethyl-3-propionate)imidazo[1,2-a]-s-triazin-4-one (I) [for preparation see Table 1 given above in Example 1] (174 mg, 0.69 mmol) in acetonitrile (6 ml) was added a solution of ammonia (28%) (3.6 mL). The mixture was stirred at 20° C. for 3 days. Then the pH of the solution was adjusted to 7-8 using additional of hydrochloric acid 1N. The solid that deposited was collected, washed with acetonitrile to give the title compound, Compound II, (112 mg) as a beige powder.

¹H NMR (DMSO-d₆) δ ppm: 2.60 (t, 2H, J=6.9 Hz, CH₂), 4.05 (t, 2H, J=6.9 Hz, CH₂), 6.91 (br, 2H, NH₂), 6.98 (br, 1H, NH), 7.17 (d, 1H, J=2.6, CH), 7.30 (d, 1H, J=2.6, CH), 7.44 (br, 1H, NH)

Mass spectrum: m/z (FAB>0) 445 (2M+H)⁺, 223 (M+H)⁺, (FAB<0) 221 (M-H)⁻

### Step B: 2-Aminoimidazo[1,2-a]-s-triazin-4-one-8-propionic acid

To a solution of 2-Amino-8-(ethyl-3-propionate)imidazo[1,2-a]-s-triazin-4-one (I) [for preparation see Table 1 given above in Example 1] (200 mg, 0.79 mmol) in a mixture of water/acetonitrile (1/1)(8 ml) was added a solution of sodium hydroxide (2M) (880 μL). The mixture was stirred at 20° C. for 3 hours. Then the pH of the solution was adjusted to 7-8 using additional of hydrochloric acid IN. The reaction mixture was evaporated to dryness. The residue was purified on reverse-phase column using water as eluant to give the title compound, Compound III, (103 mg) as a white powder.

¹H NMR (DMSO-d₆) δ ppm: 2.20 (t, 2H, J=7.0 Hz, CH₂), 3.95 (t, 2H, J=7.0 Hz, CH₂), 6.90 (br, 2H, NH₂), 7.20 (d, 1H, J=2.6, CH), 7.25 (d, 1H, J=2.6, CH)

Mass spectrum: m/z (FAB>0) 224 (M+H)⁺, (FAB<0) 222 (M-H)⁻

### Example 3.4

### 2-Amino-8-β-D-ervthrofuranosvl-imidazo[1,2-a]-s-triazin-4-one

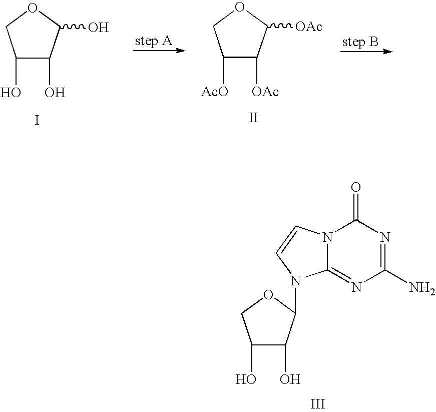

### Step A: 1,2,3-Tri-O-acetyl-D-erythrofuranose

D-erythrose (6.2 gr, 51.6 mmol) was exchanged four times with anhydrous pyridine by evaporation at 50° C. in vacuo. The residue was dissolved in pyridine (6 mL) and stirred at 0° C. Acetic anhydride was added and the mixture was stirred at 4° C. for 24 hours. The reaction mixture was evaporated to dryness. The residue was distilled under reduced pressure to give the title compound (1.93 gr) as a yellow syrup. The NMR analysis indicate a mixture of β-anomer (87%) and x-anomer (13%).

### Step B: 2-Amino-8-β-D-erythrofuranosylimidazo[1,2-a]-s-triazin-4-one

2-Aminoimidazo[1,2-a]-s-triazin-4-one [for preparation see Journal of Medicinal Chemistry, 1978, Vol 21, No 9, 883] (630 mg, 4.16 mmol) was treated under reflux with an excess of hexamethyldisilazane containing a catalytic amount of (NH₄)SO₄. The excess of hexamethyldisilazane was removed by distillation under reduced pressure. This residue was stirred in acetonitrile. A solution of 1,2,3-Tri-O-acetyl-D-erythrofuranose (1.02 gr, 4.16 mmol) in acetonitrile and trimethylsilyl trifluoromethanesulfonate (1.6 eq.) were added to the mixture and stirred at reflux for 12 hours. The reaction mixture was poured into an aqueous solution of sodium hydrogenocarbonate and then evaporated to dryness. The residue was purified on reverse-phase column using water as eluant to give the title compound (85 mg) as a beige powder.

¹H NMR (DMSO-d₆) δ ppm: 3.74 (dd, 1H, J=9.3 Hz, J=1.5 Hz), 4.17 (m, 1H), 4.28 (dd, 1H, J=9.3 Hz, J=3.7 Hz), 4.47 (m, 1H), 5.77 (d, 1H, J=6.8 Hz), 6.95 (br, 2H, NH₂), 7.39 (d, 1H, J=2.8 Hz), 7.51 (d, 1H, J=2.8 Hz)

### Example 4

This scheme illustrates the synthesis of 2-Amino-8-(2-C-methyl-β-D-ribofuranosyl)-imidazo[1,2-a]-s-triazin-4-one

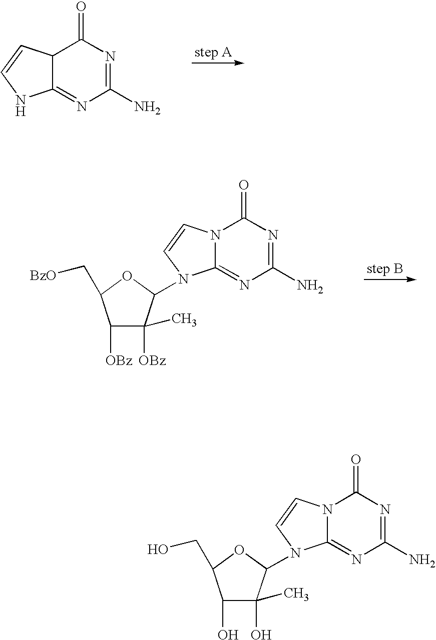

### Step A: 2-Amino-8-(2,3,5-Tri-O-benzoyl-2-C-methyl-μ-D-ribofuranosyl)-imidazo[1,2-a]-s-triazin-4-one

2-Aminoimidazo[1,2-a]-s-triazin-4-one [for preparation see Journal of Medicinal Chemistry, 1978, Vol 21, No 9. 883] (1.04 gr, 6.88 mmol) was suspended in 1,2-dichloroethane (25 mL). N,O-bis(trimethylsilyl)acetamide (3.5 mL, 14.21 mmol) were added in one portion and the mixture was heated to 60° C. for 15 hours. A solution of 1,2,3,5-Tetra-O-benzoyl-2-C-methyl-β-D-ribofuranose (3.67 gr, 6.32 mmol) and Tin(IV) chloride (0.95 μL, 8.07 mmol) were added at 20° C. Then the mixture was stirred at reflux for 4 hours and poured into an aqueous solution of sodium hydrogenocarbonate. The aqueous solution was extracted with ethyl acetate. The organic layer was evaporated to dryness. The crude product was purified on silica gel using dichloromethane/methanol (99/1) as eluant to give the title compound (1.36 g) as a beige powder.

Mass spectrum: m/z (FAB>0) 1219 (2M+H)⁺, 610 (M+H)⁺, (FAB<0) 1217 (2M-H)⁻, 608 (M-H)⁻

### Step B: 2-Amino-8-(2-C-methyl-β-D-ribofuranosyl)-imidazo[1,2-a]-s-triazin-4-one

Sodium methoxide (330 mg, 6.11 mmol) was added to a solution of compound from Step A (1.23 gr, 2.02 mmol) in methanol (20 mL) and stirred at 20° C. for 1.5 hours. Then the pH of the solution was adjusted to 7-8 using additional of acetic acid. The reaction mixture was evaporated to dryness to remove methanol. Water (40 mL) were added to the residue. The aqueous layer was washed with ethyl acetate (2×20 mL) and evaporated to dryness. The crude product was purified on silica gel reverse-phase (C18) using water as eluant to give the title compound (410 mg) as a white powder.

¹H NMR (DMSO-d₆) δ ppm: 0.87 (s, 3H, CH₃), 3.60-3.69 (m, 1H), 3.78-3.95 (m, 3H), 5.14-5.39 (m, 3H, OH), 5.78 (s, 1H), 7.0 (br, 2H, NH₂), 7.42 (d, 1H, J=2.7 Hz), 7.64 (d, 1H, J=2.7 Hz)

Mass spectrum: m/z (FAB>0) 595 (2M+H)⁺, 298 (M+H)⁺, (FAB<0) 296 (M-H)⁻.

### Example 5

### N2-(isobutyryl)-8-(isobutyryl)imidazo[1,2-a]-s-triazin-4-one

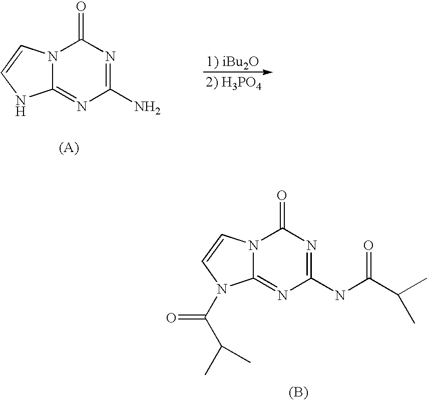

Compound (A) (500 mg) was suspended in isobutyric anhydride (13 ml). After addition of one drop of 85% phosphoric acid the reaction mixture was refluxed 2 hrs. After filtration, the excess of anhydride was evaporated under vacuo ant the residue vas treated with ice water. The precipitate was filtered off and dried (300 mg).

¹H NMR (DMSO-d₆) δ ppm: 1.09 (d, 6H, 2×CH₃), 1.23 (d, 6H, 2×CH₃), 2.94 (5-uplet, 1H, CH), 4.34 (5-uplet, 1H, CH), 7.65 (d, 1H, CH base, J=2.9 Hz), 7.78 (d, 1H, CH base, J=2.9 Hz)10.63 (s, 1H,NH)

Mass spectrum: m/z (FAB>0) 292 (M+H)⁺, 563 (2M+H)⁺;

### Example 6

### 7-Chloro-1-(methylpivalate)imidazo[1,2-a]pyrimidin-5-one

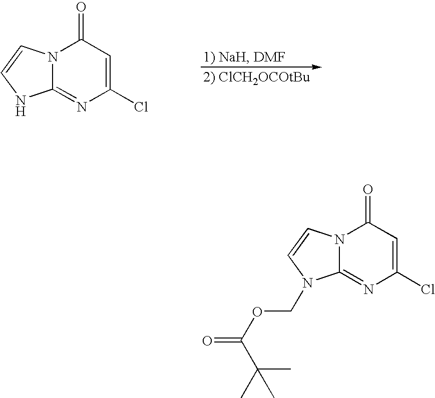

To a suspension of 7-chloroimidazo[1,2-a]pyrimidin-5-one [for preparation see Annals of the New York Academy of Sciences, 1975, 255, 166-176] (600 mg, 3.54 mmol) in dry dimethylformamide (20 mL) was added sodium hydride (60% in oil, 168 mg, 4.2 mmoles) at 20° C. and stirred for one hour. Chloromethyl pivalate (612 μL) was added and stirred at 20° C. for 16 hours. The reaction mixture was evaporated to dryness. The residue was purified on silica gel using dichloromethane/methanol (99/1) as eluant to give the title compound (710 mg) as a white powder.

¹H NMR (DMSO-d₆) δ ppm: 1.15 (s, 9H, CH₃), 6.04 (s, 2H, CH₂), 6.11 (s, 1H), 7.75 (syst AB, 2H, CH);

Mass spectrum: m/z (FAB>0) 284 (M+H)⁺, 567 (2M+H)⁺.

### 7-Chloro-1-(methylpivaloyl)imidazo[1,2-c]pyrimidin-5-one

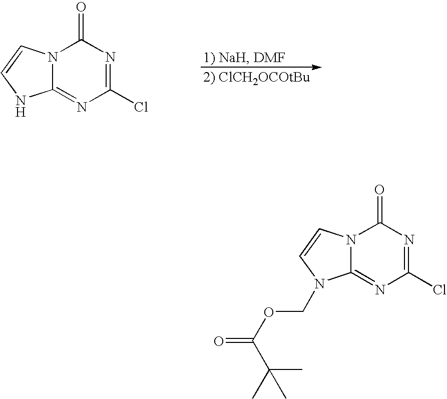

To a suspension of 7-Chloroimidazo[1,2-c]pyrimidin-5-one [for preparation see Journal of Organic Chemistry, 40(25), 1975, 3708-13] (1 g, 5.89 mmol) in dry dimethylformamide (60 mL) was added sodium hydride (60% in oil, 280 mg, 7.0 mmol) at 20° C. and stirred for one hour. Chloromethyl pivalate (1 mL) was added and stirred at 50° C. for 16 hours. The reaction mixture was evaporated to dryness. The residue was purified on silica gel using dichloromethane/methanol (99/1) as eluant to give the title compound (110 mg) as a white powder.

¹H NMR (DMSO-d₆) δ ppm: 1.13 (s, 9H, CH₃), 6.09 (s, 2H, CH₂), 7.02 (s, 1H, H8), 7.78 (syst AB, 2H, CH);

Mass spectrum: m/z (FAB>0) 284 (M+H)⁺, 567 (2M+H)⁺.

### Example 7

### Example 7.1

### 2-Amino-8-(3-deoxy-erythro-β-D-pentofuranosyl)imidazo[1,2-a]-s-triazin-4-one

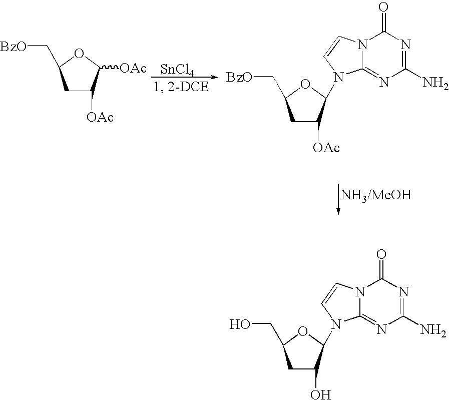

A mixture of 2-aminoimidazo[1,2-a]-s-triazin-4-one (500 mg, 1 eq), hexamethyldisilazane (10 ml), and a few crystals of ammonium sulfate was heated at reflux temperature for 12 hours. Solvent was removed under vacuo and the residual gum was used without further purification. To a solution of the above derivative in 1,2-dichloroethane (5 ml) was added 1,2-di-O-acetyl-5-O-benzoyl-3-deoxy-β-D-erythro-pentofuranose (1.2 eq) followed by SnCl₄ (1.2 eq). The reaction mixture was stirred at 80° C. for 3hrs. The solution was then poured into a saturated NaHCO₃ solution and the resulting emulsion was filtered through a Cellite pad that was washed with dichloromethane. The combined organic layer was washed with water and was dried over Na₂SO₄. The solvent was evaporated to a foam which was chromatographed on a silica gel column. The band containing the requisite product was collected and the solvent was evaporated to leave the protected nucleoside. To a solution of this nucleoside in anhydrous methanol was added sodium methylate. The reaction mixture was stirred for 12 hours and then neutralized with a HCl 1N solution. Solvent was removed under vacuo and the residue was dissolved in water. The aqueous solution was extracted with ethyl acetate (three times). The aqueous filtrate was concentrated and purified on a preparative C18 Column. The fractions containing the requisite product was collected and the solvent was evaporated to leave the nucleoside

¹H NMR (DMSO-d₆) δ ppm: 1.86 (m, 1H, H3′a), 2.10 (m, 1H, H3′b), 3.50 (m, 1H, H5′a), 3.61 (m, 1H, H5′b), 4.28 (m, 1H, H4′), 4.35 (m, 1H, H2′), 5.09 (t, 1H, OH), 5.66 (d, 1H, OH), 5.69 (d, 1H, H1′, J=2 Hz), 6.98 (s, 2H, NH₂), 7.34 (d, 1H, H base, J=2.7 Hz), 7.50 (d, 1H, H base, J=2.7 Hz)

Mass spectrum: m/z (FAB>0) 268 (M+H)⁺, 535 (2M+H)⁺

### Example 7.2

### 2-Amino-8-(4-C-hydroxymethyl-β-D-ribofuranosyl)imidazo[1,2-a]-s-triazin-4-one

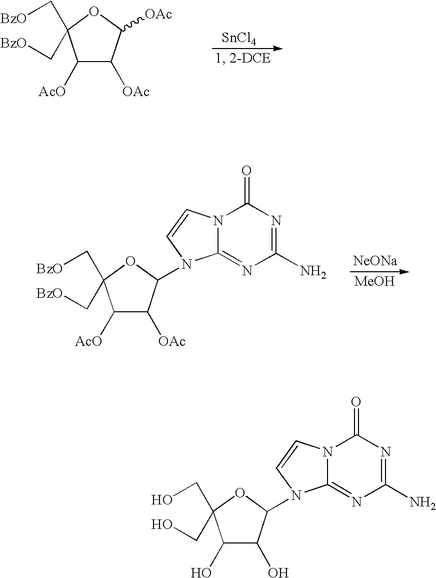

The same procedure described in Example 7.1 was used, which provided the following:

¹H NMR (DMSO-d₆) δ ppm: 3.48 (m, 4H, H5′a/b, H6′a/b), 4.07 (t, 1H, J=4.7 Hz), 4.42 (m, 1H), 4.52 (t, 1H, OH, J=5.7 Hz), 5.00 (t, 1H, OH, J=5.3 Hz), 5.07 (d, 1H, OH, J=4.3 Hz), 5.38 (d, 1H, OH, J=6.7 Hz), 5.77 (d, 1H, H1′, J=7.2 Hz), 6.91 (s, 2H, NH₂), 7.36 (d, 1H, H base, J=2.7 Hz), 7.42 (d, 1H, H base, J=2.7 Hz)

Mass spectrum: m/z (FAB>0) 314 (M+H)⁺, 627 (2M+H)⁺

### Example 7.3

### 2-Amino-8-(β-L-ribofuranosyl)imidazo[1,2-a]-s-triazin-4-one

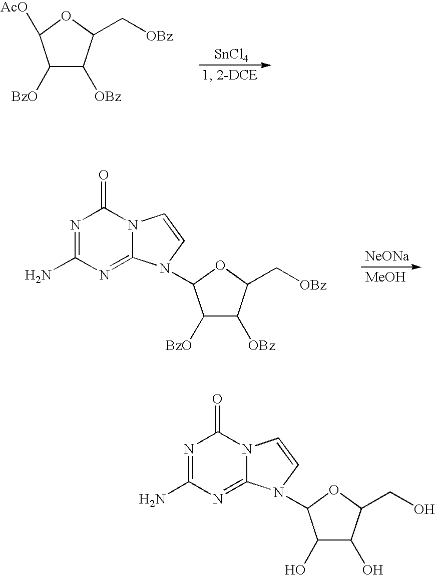

The same procedure as described in Example 7.1 was used, and the following data was obtained:

¹H NMR (DMSO-d₆) δ ppm: 3.52 (m, 2H, H5′a/b), 3.84 (m, 1H, H4′), 4.01 (m, 1H, H3′), 4.23 (m, 1H, H2′), 5.03 (t, 1H, OH), 5.13 (d, 1H, OH), 5.43 (d, 1H, OH), 5.72 (d, 1H, H1′, J=5.9 Hz), 6.92 (s, 2H, NH₂), 7.35 (d, 1H, H base, J=2.7 Hz), 7.44 (d, 1H, H base, J=2.7 Hz)

Mass spectrum: m/z (FAB>0) 284 (M+H)⁺

### Example 7.4

### 2-Amino-8-(β-D-xylofuranosyl)imidazo[1,2-a]-s-triazin-4-one

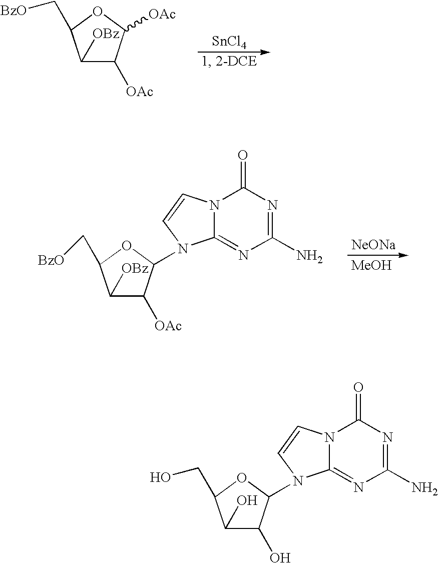

The final product was prepared by the same procedure as in Example 7.1, with the following results:

¹H NMR (DMSO-d₆) δ ppm: 3.69 (m, 2H, H5′a/b), 4.04 (m, 1H), 4.14 (m, 2H), 4.77 (t, 1H, OH, J=5.5 Hz), 5.62 (d, 1H, OH, J=3.9 Hz), 5.77 (d, 1H, H1′, J=2.0 Hz) 5.88 (d, 1H, OH, J=4.1 Hz), 6.99 (s, 2H, NH₂), 7.35 (d, 1H, H base, J=2.7 Hz), 7.44 (d, 1H, H base, J=2.7 Hz)

Mass spectrum: m/z (FAB>0) 284 (M+H)⁺

### Example 7.5

### 2-Amino-8-(4-C-hydroxymethyl-3-C-methyl-β-D-ribofuranosyl)imidazo[1,2-a]-s-triazin-4-one

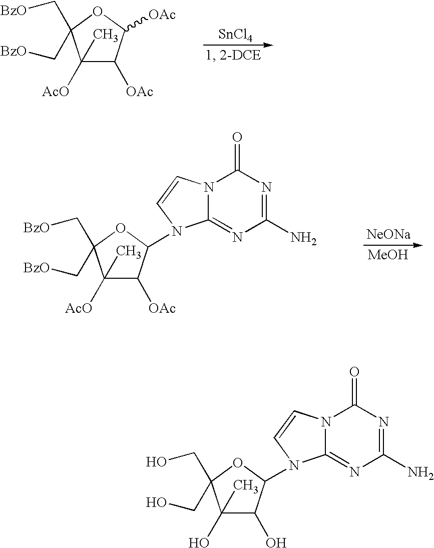

A procedure identical to that in Example 7.1 was followed.

¹H NMR (DMSO-d₆) δ ppm: 1.25 (s, 3H, CH3), 3.46 (dd, 1H, H5′a), 3.56 (m, 3H, H5′a, H6′a/b), 4.32 (m, 1H), 4.46 (t, 1H, OH), 4.6 (s, 1H, OH), 5.14 (t, 1H, OH), 5.39 (d, 1H, OH), 5.74 (d, 1H, H1′, J=7.9 Hz), 6.92 (s, 2H, NH₂), 7.38 (d, 1H, H base, J=2.7 Hz), 7.48 (d, 1H, H base, J=2.7 Hz)

Mass spectrum : m/z (FAB>0) 328 (M+H)⁺

### Example 7.6

### 2-Amino-8-(β-D-allofuranosyl)imidazo[1,2-a]-s-triazin-4-one

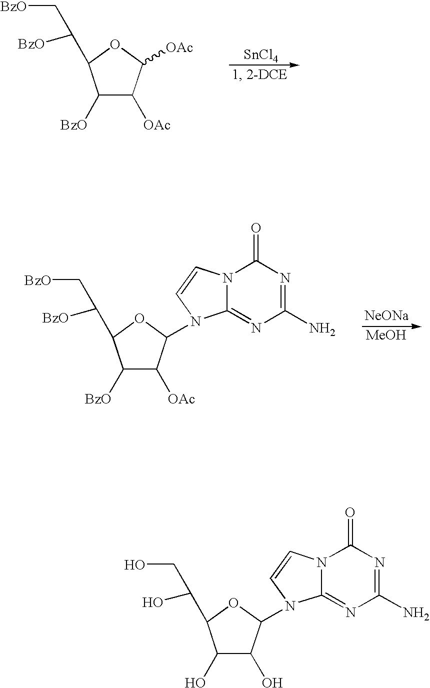

The title compound was prepared according to the procedure given in Example 7.1.

¹H NMR (DMSO-d₆) δ ppm: 3.40 (m, 4H), 3.61 (m, 1H), 3.91 (m, 1H), 4.13 (m, 1H), 4.29 (m, 1H), 4.64 (t, 1H, OH, J=5.5 Hz), 5.11 (d, 1H, OH, J=4.1 Hz), 5.16 (d, 1H, OH, J=5.1 Hz), 5.42 (d, 1H, OH, J=6.0 Hz), 5.76 (d, 1H, H1′, J=4.8 Hz), 6.97 (s, 2H, NH₂), 7.42 (d, 1H, H base, J=2.7 Hz), 7.45 (d, 1H, H base, J=2.7 Hz)

Mass spectrum: m/z (FAB>0) 152 (BH₂)⁺, 314 (M+H)⁺, 627 (2M+H)⁺

### Example 7.7

### 2-Amino-9-(5-deoxy-5-iodo-β-D-ribofuranosyl)imidazo[1,2-a]-s-triazin4-one

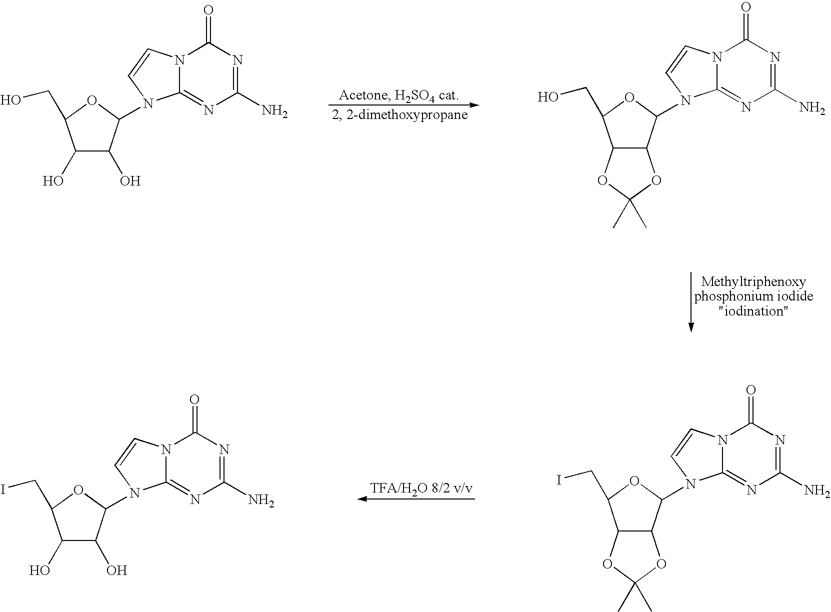

¹H NMR (DMSO-d₆) δ ppm: 3.6 (m, 2H, H5′a/b), 3.94 (m, 1H, H4′), 4.02 (m, 1H, H3′), 4.50 (m, 1H, H2′), 5.50 (s1, 1H, OH), 4.74 (t, 1H, OH, J=5.1 Hz), 5.65 (d, 1H, OH), 5.81 (d, 1H, H1′, J=6.3 Hz), 7.18 (s1, 1H, NH₂), 7.37 (s, 2H, NH₂), 7.52 (syst AB, 2H, CH base)

Mass spectrum: m/z (FAB>0) 394 (M+H)⁺, 787 (2M+H)⁺

### Example 7.8

### 2-Amino-9-(5-deoxy-5-azido-β-D-ribofuranosyl)imidazo[1,2-a]-s-triazin-4-one

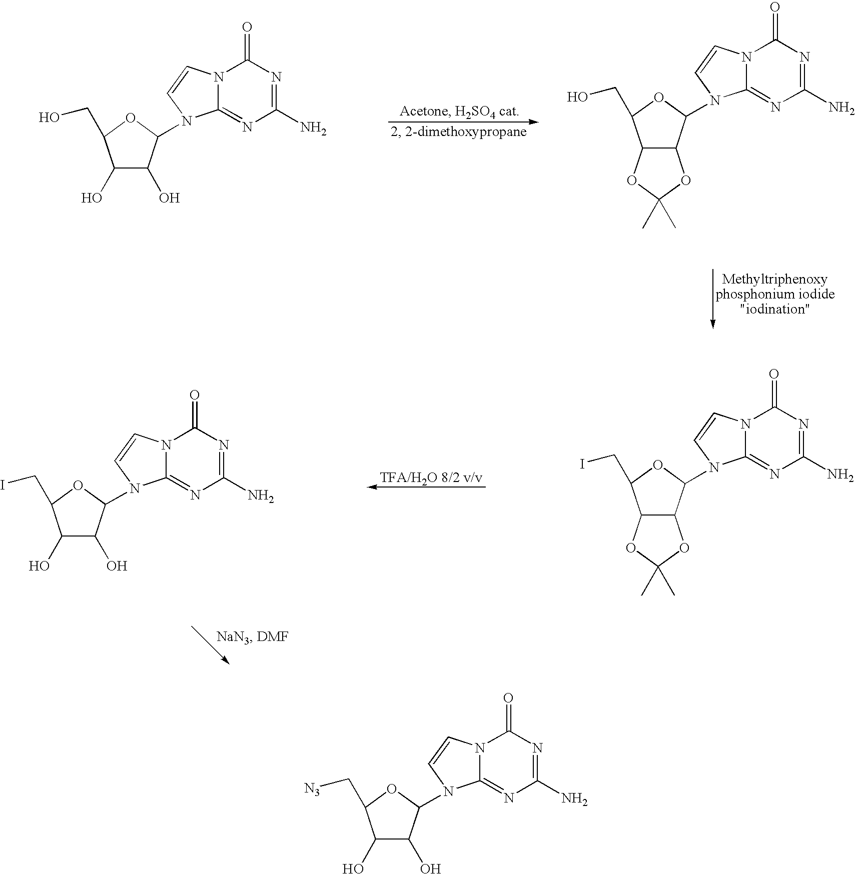

This example utilizes a process identical to that of Example 7.7 but adds a single step to exchange the iodo group for an azido group, thereby forming the title compound.

¹H NMR (DMSO-d₆) δ ppm: 3.66 (m, 2H, H5′a/b), 4.10 (m, 2H, H3′, H4′), 4.44 (m, 1H, H2′), 5.41 (d, 1H, OH), 5.65 (d, 1H, OH), 5.80 (d, 1H, H1′, J=5.8 Hz), 7.03 (s, 2H, NH₂), 7.43 (d, 1H, CH base), 7.49 (d, 1H, CH base)

Mass spectrum: m/z (FAB>0) 309 (M+H)⁺

### Example 7.9

### N2-Benzoyl-8-(5-acetylmercapto-5-deoxy-β-D-ribofuranosyl)imidazo-[1,2-a]-s-triazin-4-one

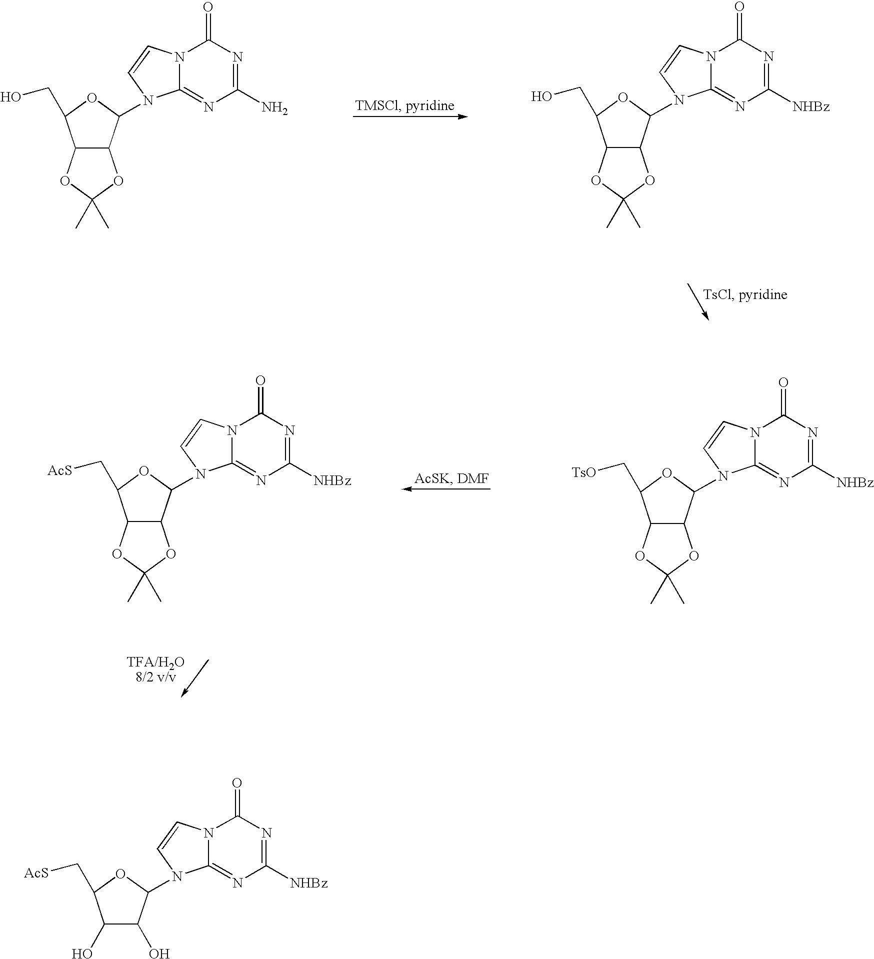

¹H NMR (DMSO-d₆) δ ppm: 2.36 (s, 3H, CH₃), 3.2 (m, 2H, H5′a/b), 3.94 (m, 1H, H4′), 4.10 (m, 1H, H3′), 4.68 (m, 1H, H2′), 5.47 (d, 1H, OH, J=3.3 Hz), 5.68 (d, 1H, OH, J=4.5 Hz), 5.88 (d, 1H, H1′, J=6.1 Hz), 7.4-7.6 (m, 3H, Bz), 7.73 (dd, 1H, CH base, J =3.0 Hz), 7.80 (dd, 1H, CH base, J=3.0 Hz), 10.96 (s, 1H, NH)

Mass spectrum: m/z (FAB>0) 446 (M+H)⁺

### Example 7.10

### 2-Amino-8-(5-mercapto-5-deoxy-β-D-ribofuranosyl)imidazo[1,2-a]-pyrimidine

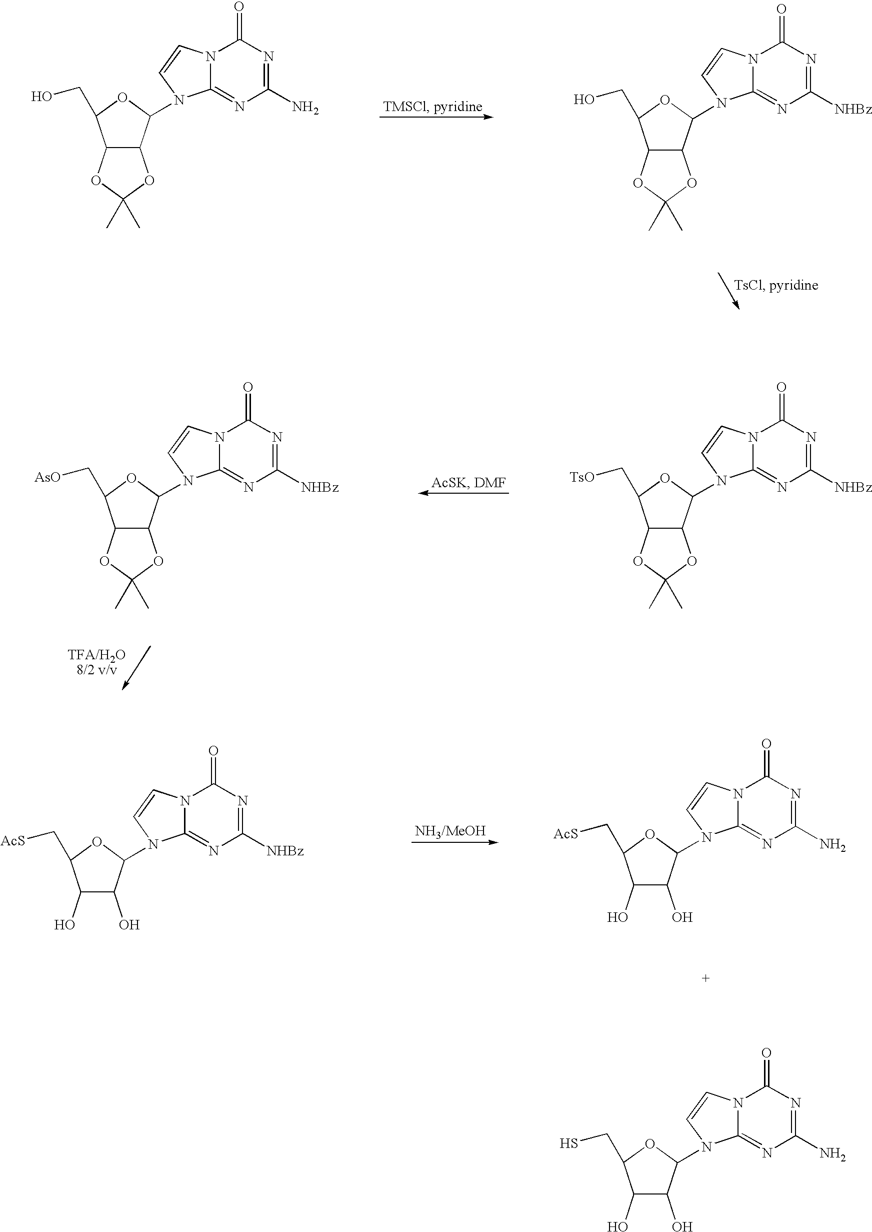

¹H NMR (DMSO-d₆) δ ppm: 2.36 (s, 3H, CH₃), 3.04 (m, 2H, H5′a/b), 4.07 (m, 2H, H3′, H4′), 4.45 (m, 1H, H2′), 5.43 (d, 1H, OH, J=3.7 Hz), 5.62 (d, 1H, OH, J=5.1 Hz), 5.79 (d, 1H, H1′, J=6.6 Hz), 7.01 (s, 2H, NH₂), 7.47 (syst AB, 2H, CH base)

Mass spectrum: m/z (FAB>0) 152 (BH₂)⁺, 342 (M+H)⁺

### Example 7.11

### 2-Amino-8-(5-acetylmercapto-5-deoxy-β-D-ribofuranosyl)imidazo-[1,2-a]-pyrimidine (Shown in the Schematic for Example 7.10 as one of Two Final Products)

¹H NMR (DMSO-d₆) δ ppm: 2.22 (m, 2H, H5′a/b), 3.13 (m, 1H, H4′), 3.23 (m, 1H, H3′), 3.54 (q, 1H, H2′), 4.25 (d, 1H, OH), 4.44 (d, 1H, OH), 4.63 (d, 1H, H1, J=6.6 Hz), 7.08 (s, 2H, NH₂), 7.42 (d, 1H, CH base), 7.47 (d, 1H, CH base)

Mass spectrum: m/z (FAB>0) 300 (M+H)⁺

### Example 7.12

### 2(R)-[1-(2-Aminoimidazo[1,2-a]-s-triazin-4-on-8-yl)-2-hydroxy-ethoxy]-1,3-propanediol

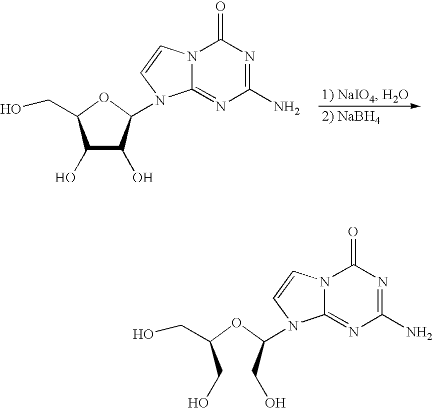

NaIO₄ (491 mg) was added to a stirred suspension of adenosine and guanosine (500 mg) in water at room temperature. After 3 h, NaBH₄ (177 mg) was added. After an additional 1.5 h, the pH was reduced from 9.5 to 7.0 with concentrated HCl. Acetone was added and the reaction mixture was stirred 24 hrs. Solvents were removed under vacuum and the residue was purified by a silica gel column (eluent DCM/MeOH 8/2) to afford the title compound.

¹H NMR (DMSO-d₆) δ ppm: 3.2-3.8 (m, 8H), 4.48 (t, 1H, OH, J=5.7 Hz), 4.74 (t, 1H, OH, J=5.1 Hz), 5.14 (t, 1H, OH, J=6.0 Hz), 5.73 (dd, 1H, J=5.1 Hz, J=6.7 Hz), 6.94 (s, 2H, NH₂), 7.36 (syst AB, 2H, CH base)

Mass spectrum: m/z (FAB>0) 286 (M+H)⁺, 570 (2M+H)⁺

### Example 7.13

### 2(R)-[1-(2-Aminoimidazo[1,2-a]-s-triazin-4-on-8-yl)-2-hydroxy-ethoxy]-1-propanol

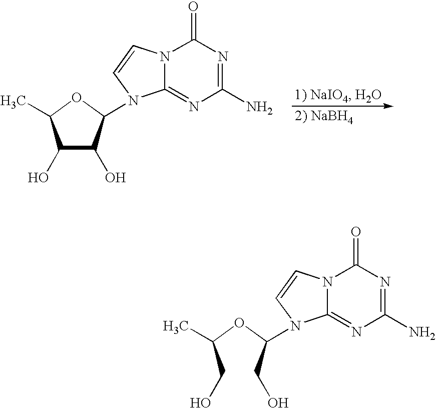

The same process as used in Example 7.12 was used in this example to provide the title compound.

¹H NMR (DMSO-d₆) δ ppm: 0.85 (t, 3H, CH₃, J=6.8), 1.28 (m, 2H, CH₂), 1.71 (m, 2H, CH₂), 3.86 (t, 2H, CH₂, J=7.1 Hz), 6.86 (s, 2H, NH₂), 7.32 (syst AB, 2H, CH base)

Mass spectrum: m/z (FAB>0) 270 (M+H)⁺, 539 (2M+H)⁺

### Example 8

### Assessment of Biological Activity

Compounds can exhibit anti-flavivirus or pestivirus activity by inhibiting flavivirus or pestivirus polymerase, by inhibiting other enzymes needed in the replication cycle, or by other pathways.

The test compounds are dissolved in DMSO at an initial concentration of 200 μM and then serially diluted in culture medium.

Unless otherwise stated, baby hamster kidney (BHK-21) (ATCC CCL-10) and Bos Taurus (BT) (ATCC CRL 1390) cells are grown at 37° C. in a humidified CO₂ (5%) atmosphere. BHK-21 cells are passaged in Eagle MEM additioned of 2 mM L-glutamine, 10% fetal bovine serum (FBS, Gibco) and Earle's BSS adjusted to contain 1.5 g/L sodium bicarbonate and 0.1 mM non-essential amino acids. BT cells are passaged in Dulbecco's modified Eagle's medium with 4 mM L-glutamine and 10% horse serum (HS, Gibco), adjusted to contain 1.5 g/L sodium bicarbonate, 4.5 g/L glucose and 1.0 mM sodium pyruvate. The vaccine strain 17D (YFV-17D) (Stamaril®, Pasteur Merieux) and Bovine Viral Diarrhea virus (BVDV) (ATCC VR-534) are used to infect BHK and BT cells, respectively, in 75 cm² bottles. After a 3 day incubation period at 37° C., extensive cytopathic effect can be observed. Cultures are freeze-thawed three times, cell debris are removed by centrifugation and the supernatant aliquoted and stored at −70° C. YFV-17D and BVDV are titrated in BHK-21 and BT cells, respectively, that were grown to confluency in 24-well plates.

### Example 8.1

### Phosphorylation Assay of Nucleoside to Active Triphosphate

To determine the cellular metabolism of the compounds, HepG2 cells are obtained from the American Type Culture Collection (Rockville, Md.), and are grown in 225 cm² tissue culture flasks in minimal essential medium supplemented with non-essential amino acids, 1% penicillin-streptomycin. The medium is renewed every three days, and the cells are subcultured once a week. After detachment of the adherent monolayer with a 10 minute exposure to 30 mL of trypsin-EDTA and three consecutive washes with medium, confluent HepG2 cells are seeded at a density of 2.5×10⁶ cells per well in a 6-well plate and exposed to 10 μM of [³H] labeled active compound (500 dpm/pmol) for the specified time periods. The cells are maintained at 37° C. under a 5% CO₂ atmosphere. At the selected time points, the cells are washed three times with ice-cold phosphate-buffered saline (PBS). Intracellular active compound and its respective metabolites are extracted by incubating the cell pellet overnight at −20° C. with 60% methanol followed by extraction with an additional 20 μL of cold methanol for one hour in an ice bath. The extracts are then combined, dried under gentle filtered air flow and stored at −20° C. until HPLC analysis.

### Example 8.2

### Bioavailability Assay in Cynomolgus Monkeys

Within 1 week prior to the study initiation, the cynomolgus monkey is surgically implanted with a chronic venous catheter and subcutaneous venous access port (VAP) to facilitate blood collection and underwent a physical examination including hematology and serum chemistry evaluations and the body weight was recorded. Each monkey (six total) receives approximately 250 μCi of ³H activity with each dose of active compound at a dose level of 10 mg/kg at a dose concentration of 5 mg/mL, either via an intravenous bolus (3 monkeys, IV), or via oral gavage (3 monkeys, PO). Each dosing syringe is weighed before dosing to gravimetrically determine the quantity of formulation administered. Urine samples are collected via pan catch at the designated intervals (approximately 18-0 hours pre-dose, 0-4, 4-8 and 8-12 hours post-dosage) and processed. Blood samples are collected as well (pre-dose, 0.25, 0.5, 1, 2, 3, 6, 8, 12 and 24 hours post-dosage) via the chronic venous catheter and VAP or from a peripheral vessel if the chronic venous catheter procedure should not be possible. The blood and urine samples are analyzed for the maximum concentration (Cₘₐₓ), time when the maximum concentration is achieved (Tₘₐₓ), area under the curve (AUC), half life of the dosage concentration (T_{1/2}), clearance (CL), steady state volume and distribution (Vₛₛ) and bioavailability (F).

### Example 8.3

### Bone Marrow Toxicity Assay

Human bone marrow cells are collected from normal healthy volunteers and the mononuclear population are separated by Ficoll-Hypaque gradient centrifugation as described previously by Sommadossi J-P, Carlisle R. “Toxicity of 3′-azido-3′-deoxythymidine and 9-(1,3-dihydroxy-2-propoxymethyl)guanine for normal human hematopoietic progenitor cells in vitro” Antimicrobial Agents and Chemotherapy 1987; 31:452-454; and Sommadossi J-P, Schinazi R F, Chu C K, Xie M-Y. “Comparison of cytotoxicity of the (−)- and (+)-enantiomer of 2′,3′-dideoxy-3′-thiacytidine in normal human bone marrow progenitor cells” Biochemical Pharmacology 1992; 44:1921-1925. The culture assays for CFU-GM and BFU-E are performed using a bilayer soft agar or methylcellulose method. Drugs are diluted in tissue culture medium and filtered. After 14 to 18 days at 37° C. in a humidified atmosphere of 5% CO₂ in air, colonies of greater than 50 cells are counted using an inverted microscope. The results are presented as the percent inhibition of colony formation in the presence of drug compared to solvent control cultures.

### Example 8.4

### Mitochondria Toxicity Assay

HepG2 cells are cultured in 12-well plates as described above and exposed to various concentrations of drugs as taught by Pan-Zhou X-R, Cui L, Zhou X-J, Sommadossi J-P, Darley-Usmer V M. “Differential effects of antiretroviral nucleoside analogs on mitochondrial function in HepG2 cells” Antimicrob Agents Chemother 2000; 44:496-503. Lactic acid levels in the culture medium after 4 day drug exposure are measured using a Boehringer lactic acid assay kit. Lactic acid levels are normalized by cell number as measured by hemocytometer count.

### Example 8.5

### Cytotoxicity Assay

Cells are seeded at a rate of between 5×10³ and 5×10⁴/well into 96-well plates in growth medium overnight at 37° C. in a humidified CO₂ (5%) atmosphere. New growth medium containing serial dilutions of the drugs is then added. After incubation for 4 days, cultures are fixed in 50% TCA and stained with sulforhodamineB. The optical density was read at 550 nm. The cytotoxic concentration was expressed as the concentration required to reduce the cell number by 50% (CC₅₀).

### Example 8.6

### Cell Protection Assay (CPA)

The assay is performed essentially as described by Baginski, S. G.; Pevear, D. C.; Seipel, M.; Sun, S. C. C.; Benetatos, C. A.; Chunduru, S. K.; Rice, C. M. and M. S. Collett “Mechanism of action of a pestivirus antiviral compound” PNAS USA 2000, 97(14), 7981-7986. MDBK cells (ATCC) are seeded onto 96-well culture plates (4,000 cells per well) 24 hours before use. After infection with BVDV (strain NADL, ATCC) at a multiplicity of infection (MOI) of 0.02 plaque forming units (PFU) per cell, serial dilutions of test compounds are added to both infected and uninfected cells in a final concentration of 0.5% DMSO in growth medium. Each dilution is tested in quadruplicate. Cell densities and virus inocula are adjusted to ensure continuous cell growth throughout the experiment and to achieve more than 90% virus-induced cell destruction in the untreated controls after four days post-infection. After four days, plates are fixed with 50% TCA and stained with sulforhodamine B. The optical density of the wells is read in a microplate reader at 550 nm. The 50% effective concentration (EC₅₀) values are defined as the compound concentration that achieved 50% reduction of cytopathic effect of the virus.

### Example 8.7

### Plaque Reduction Assay

For each compound the effective concentration is determined in duplicate 24-well plates by plaque reduction assays. Cell monolayers are infected with 100 PFU/well of virus. Then, serial dilutions of test compounds in MEM supplemented with 2% inactivated serum and 0.75% of methyl cellulose are added to the monolayers. Cultures are further incubated at 37° C. for 3 days, then fixed with 50% ethanol and 0.8% Crystal Violet, washed and air-dried. Then plaques are counted to determine the concentration to obtain 90% virus suppression.

### Example 8.8

### Yield Reduction Assay

For each compound the concentration to obtain a 6-log reduction in viral load is determined in duplicate 24-well plates by yield reduction assays. The assay is performed as described by Baginski, S. G.; Pevear, D. C.; Seipel, M.; Sun, S. C. C.; Benetatos, C. A.; Chunduru, S. K.; Rice, C. M. and M. S. Collett “Mechanism of action of a pestivirus antiviral compound” PNAS USA 2000, 97(14), 7981-7986, with minor modifications. Briefly, MDBK cells are seeded onto 24-well plates (2×105 cells per well) 24 hours before infection with BVDV (NADL strain) at a multiplicity of infection (MOI) of 0.1 PFU per cell. Serial dilutions of test compounds are added to cells in a final concentration of 0.5% DMSO in growth medium. Each dilution is tested in triplicate. After three days, cell cultures (cell monolayers and supernatants) are lysed by three freeze-thaw cycles, and virus yield is quantified by plaque assay. Briefly, MDBK cells are seeded onto 6-well plates (5×105 cells per well) 24 h before use. Cells are inoculated with 0.2 mL of test lysates for 1 hour, washed and overlaid with 0.5% agarose in growth medium. After 3 days, cell monolayers are fixed with 3.5% formaldehyde and stained with 1% crystal violet (w/v in 50% ethanol) to visualize plaques. The plaques are counted to determine the concentration to obtain a 6-log reduction in viral load.

Representative data is provided in Table 2.

TABLE 2

| | Yellow Fever | | BVDV | | DENV-2 | | West Nile | | MT-4 |
|---|---|---|---|---|---|---|---|---|---|
| | EC₅₀ | CC₅₀ | EC₅₀ | CC₅₀ | EC₅₀ | CC₅₀ | EC₅₀ | CC₅₀ | EC₅₀ |
| Compounds | μM | μM | M | μM | μM | μM | μM | μM | μM |
| 2-amino-8-(β- | >100 | >100 | 21 | >100 | >100 | >100 | >100 | >100 | >100 |
| D-2-deoxyribo- | | | 27 | | | | | | |
| furanosyl)- | | | | | | | | | |
| imidazo[1,2-a]- | | | | | | | | | |
| s-triazin-4-one | | | | | | | | | |
| 2-amino-8-(β- | >100 | >100 | 31 | >100 | >100 | >100 | >100 | >100 | >100 |
| D-ribo- | | | 33 | | | | | | |
| furanosyl)- | | | | | | | | | |
| imidazo[1,2-a]- | | | | | | | | | |
| s-triazin-4-one | | | | | | | | | |

This invention has been described with reference to its typically embodiments. Variations and modifications of the invention will be obvious to those skilled in the art from the foregoing detailed description of the invention. It is intended that all of these variations and modifications be included within the scope of this invention.

We claim:

1. A method of treating a host infected with a flavivirus or pestivirus, comprising administering an effective amount of a biologically active compound to a host in need thereof, wherein the biologically active compound has the structure of Formulae (i), (ii), (iii), (iv), (v) or (vi): 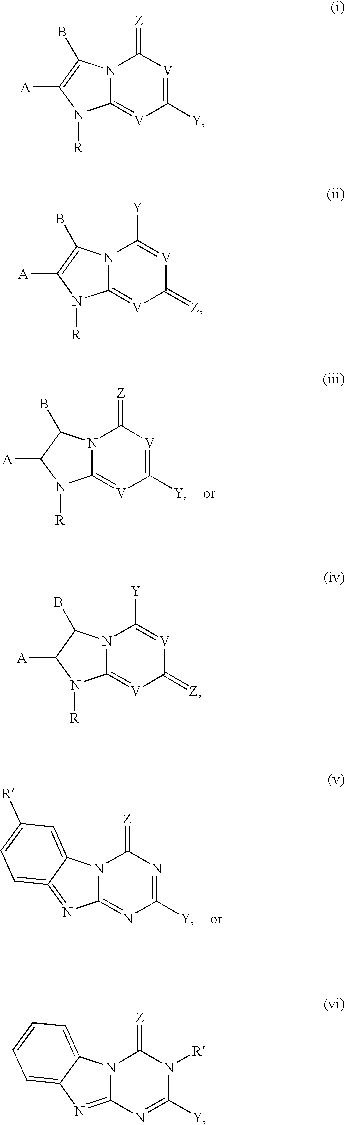
or a pharmaceutically acceptable salt or prodrug thereof, wherein:
A, B and Y, each independently, is H; halogen; OR′, S(O)ₙ; S(O)ₙR′; S(O)ₙR′R″; NR′R″; NR; CN; CF₃; CR′R″; C(═W)OR′; C(═W)SR′; C(═W)NR′R′; C₁₋₄ alkylamino; di(C₁₋₄ alkyl)amino; C₃₋₆ cycloalkylamino; NO₂; N₃; C₁₋₁₀ cyclic or acyclic, branched or unbranched alkyl, alkenyl, alkynyl; aryl; aralkyl; heterocycle; or A and B taken together with the carbon atoms to which they are attached may form a 4-7 membered carbocyclic or heterocyclic ring;
Z is O, S, NR′, or CR′R″;
each V is independently N or CR′;
each R′ and R″ independently is H; C₁₋₁₀ cyclic or acyclic, branched or unbranched alkyl, alkenyl, alkynyl; halo; O-alkyl; NH₂; NHMe; N(Me)₂; CN; acyl; aryl; heteroaryl; heterocycle; carbocycle; amino acid residue; or together with the atoms to which they are attached may form a 3-7 membered carbocyclic or heterocyclic ring;
each W is independently O, S, or NR′;
each R is independently H; C₁₋₁₀ cyclic or acyclic, branched or unbranched alkyl, alkenyl, alkynyl, acyl, aryl, or aralkyl, any of which optionally may have one or more heteroatoms and any of which may be taken alone or in combination with one another; 3-7 membered carbocycle or heterocycle; or a functional group that dissociates to provide the base where R is H;
each n is independently 0, 1 or 2;
wherein any branched or unbranched, cyclic or acyclic alkyl, alkenyl, or alkynyl may optionally comprise at least one heteroatom selected from the group consisting of O, S, N and P;
wherein any branched or unbranched, cyclic or acyclic alkyl, alkenyl, alkynyl; aryl; acyl; or aralkyl may optionally be substituted with one or more of OR′, SR′, NR′R″, halogen, NO₂, CN, N₃, CF₃, C(═W)OR′, C(═W)NR′R″, C(═W)SR′, alkyl, alkenyl, alkynyl, aryl, aralkyl, acyl, heterocycle or heteroatom selected from the group consisting of O, S, N and P; and
all tautomeric, enantiomeric and stereoisomeric forms thereof,
with the caveat that in Formula (i), when A and B are both H, both Vs are N, Z is O, and Y is —NH₂, then Formula (i) is not β-D-2′-deoxy-5-aza-7-deazaguanosine, β-D-5-aza-7-deazaguanosine, β-D-5′-methyl-5-aza-7-deazaguanosine, or 2-amino-8-(methyl-pivalate)imidazo[1,2-a]-s-triazin-4-one.

2. The method of claim 1 wherein substituent R is selected from the group consisting H; C₁₋₁₀ cyclic or acyclic, branched or unbranched alkyl, alkenyl, alkynyl; acyl; aryl; aralkyl; 3-7 membered carbocycle or heterocycle; 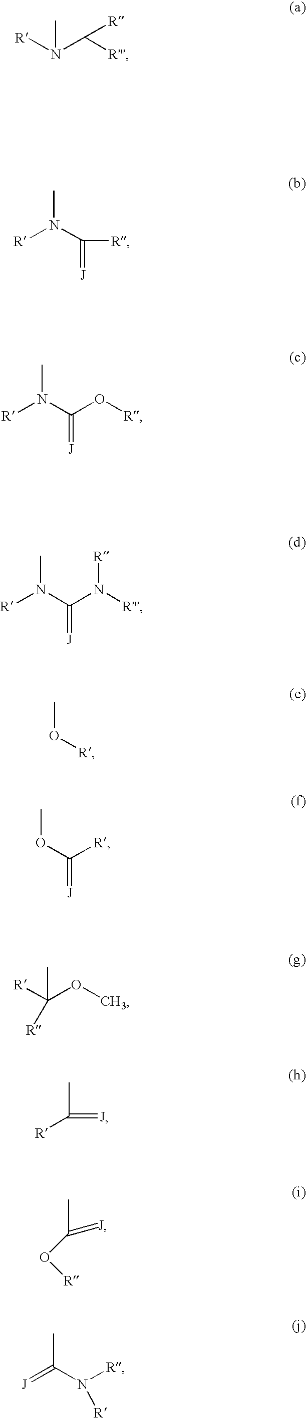 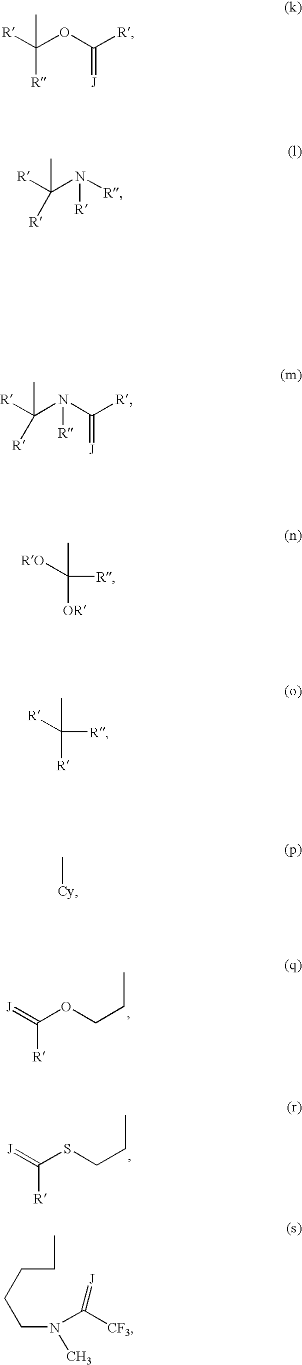 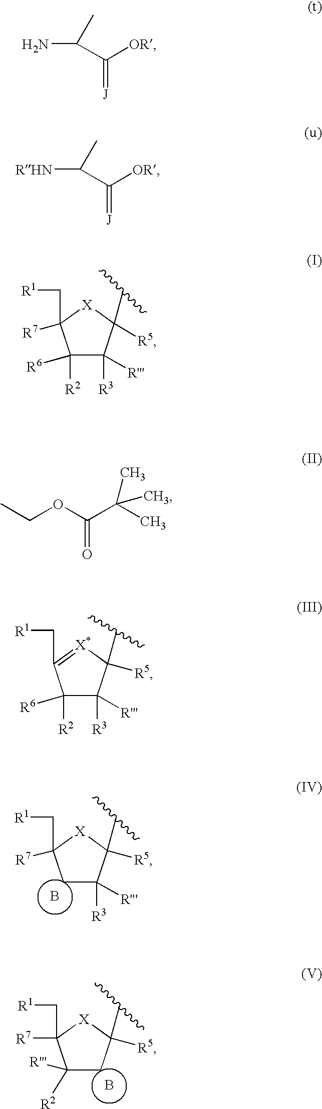 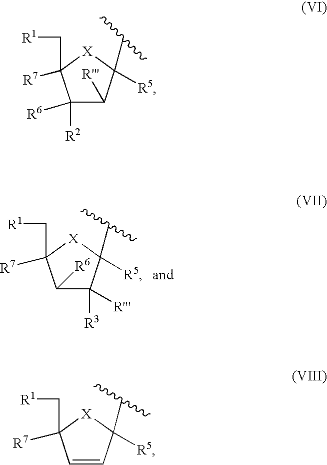
wherein:
J is O, S or N—R′;
Cy is any optionally substituted carbocycle, heterocycle or heteroaryl; and
each R′ and R″ independently is H; C₁₋₁₀ cyclic or acyclic, branched or unbranched alkyl, alkenyl, alkynyl; halo; O-alkyl; NH₂; NHMe; N(Me)₂; CN; halo; O-alkyl; NH₂; NHMe; N(Me)₂; CN; heterocycle; carbocycle; or together with the atoms to which they are attached may form a 3-7 membered carbocyclic or heterocyclic ring;
R¹ is OH, phosphate or phosphonate (including mono-, di-, or triphosphate or a stabilized phosphate prodrug); acyl (including lower acyl); alkyl (including lower alkyl); sulfonate ester including alkyl or arylalkyl sulfonyl including methanesulfonyl and benzyl, wherein the phenyl group is optionally substituted with one or more substituents as described in the definition of an aryl given herein; optionally substituted arylsulfonyl; a lipid, including a phospholipid; an amino acid; a carbohydrate; a peptide; or cholesterol, of which any of the foregoing may be O-linked at the 5′-position on the ring structure; or other pharmaceutically acceptable leaving group that, in vivo, provides a compound wherein R¹ is independently OH or O-phosphate;
each R² and R³ independently is H, OH, halo, NO₂, NH₂, N₃, CH₂N₃, CH₂NH₂, CN, CH₂CN, CH₂N₃, CH₂NHCH₃, CH₂N(CH₃)₂, CH₂OH, halogenated alkyl, alkoxy, CF₃, C(A′)₃, 2-Br-ethyl, CH₂F, CH₂Cl, CH₂CF₃, CF₂CF₃, CH₂(A′), C(A′)₂(A′)₃, SCN, OCN, NCO, haloalkenyl, Br-vinyl, haloalkynyl; —(CH₂)ₘC(O)OR⁴, —(CH₂)ₘC(O)SR⁴; —O(alkenyl), CF₃, halogen, —(CH₂)ₘNHR⁴, —(CH₂)ₘN(R⁴)₂, —(CH₂)ₘC(O)NHR⁴, —(CH₂)ₘC(O)N(R⁴)₂, —C(O)OR⁴, —O(R⁴), an optionally substituted carbocycle (typically a 3-7 membered carbocyclic ring such as, for example, a C₃₋₇ cycloalkylamino), an optionally substituted heterocycle (typically a 3-7 membered heterocyclic ring having one or more O, S and/or N), an optionally substituted heteroaryl (typically a heteroaromatic ring having one or more O, S and/or N atoms), a C₃₋₇ cycloalkylamino, and where CF₃, mercapto, optionally substituted C₁₋₄ alkyl, C₁₋₁₂ alkoxy, C₂₋₄alkenyl, or C₂₋₄ alkynyl, C₂₋₆ alkenyloxy, C₁₋₄ alkylthio, C₁₋₈ alkylcarbonyloxy, aryloxycarbonyl, C₁₋₄ alkylamino, di(C₁₋₄ alkyl)amino, Br-vinyl, —C(O)O(alkyl), O-phosphate or O-phosphonate (including mono-, di-, or triphosphate or a stabilized phosphate prodrug); O-acyl (including lower acyl); O-alkyl (including lower alkyl); O-sulfonate ester including O-alkyl or O-arylalkyl sulfonyl including O-methanesulfonyl and O-benzyl, wherein the phenyl group is optionally substituted with one or more substituents as described in the definition of an aryl given herein; —OC(O)O-aryl; —OC(O)O-aralkyl; —S(acyl); —S(alkyl); —S(alkenyl); optionally substituted O-arylsulfonyl; an O-linked lipid, including an O-phospholipid; an O-linked amino acid; an O-linked carbohydrate; an O-linked peptide; O-linked cholesterol; or other O-linked pharmaceutically acceptable leaving group that in vivo provides a compound wherein R¹ is independently H or phosphate;
each R⁴ is independently H, alkyl, alkenyl, alkynyl, acyl, aryl or aralkyl;
each R⁵ and R⁶, independently, is H, —OH, —SH, —NH₂, —CF₃, Cl, F, Br, I, optionally substituted alkyl, optionally substituted alkenyl or alkynyl, —CH₂OH, alkoxy, CH₂F, CH₂N₃, CH₂CN, —(CH₂)ₘC(O)OR⁴, —(CH₂)ₘC(O)NHR⁴, —(CH₂)ₘC(O)N(R⁴)₂, —NH(alkyl), —N(alkyl)₂, —NH(acyl), —N(acyl)₂, or C₃₋₇ cycloalkylamino;
R⁷ is H, —OR¹, —OH, —NO₂, —CF₃, —NH₂, Cl, F, Br, I, N₃, CN, optionally substituted alkyl, optionally substituted alkenyl or alkynyl, Br-vinyl, —CH₂OH, —O(R⁴), alkoxy, —(CH₂)ₘC(O)O(R⁴), —OC(O)O-aryl, —OC(O)O-aralkyl, —SR₄, —(CH₂)ₘNHR⁴, —(CH₂)ₘN(R⁴)₂, or C₃₋₇ cycloalkylamino;
X is O, S, SO₂, CH₂, CHOH, CH-halogen, C-(halogen)₂;
X* is CH, C—OH, or C-halogen;
each m is independently 0, 1 or 2;
R′″ is H, OH, SH, halo, optionally substituted C₁₋₄ alkyl, optionally substituted C₂₋₄ alkenyl or C₂₋₄ alkynyl, N₃, CN, CH₂CN, CH₂N₃, CH₂NH₂, CH₂NHCH₃, CH₂N(CH₃)₂, CH₂OH, halogenated alkyl, alkoxy, CF₃, C(A′)₃, 2-Br-ethyl, CH₂F, CH₂Cl, CH₂CF₃, CF₂CF₃, CH₂(A′), C(A)₂(A′)₃, haloalkenyl, Br-vinyl, haloalkynyl; —(CH₂)ₘC(O)OR⁴, —O(acyl), —O(lower acyl), —O(alkyl), —O(lower alkyl), —O(alkenyl), CF₃, halogen, —NO₂, —NH₂, —(CH₂)ₘNHR⁴, —(CH₂)ₘN(R⁴)₂, —(CH₂)ₘC(O)NHR⁴, —(CH₂)ₘC(O)N(R⁴)₂, or C₃₋₇ cycloalkylamino, and where the optional substitutions on alkyl, alkenyl and/or alkynyl may be one or more halogen, hydroxy, amino, alkoxy, or alkylthio groups or atoms taken in any combination; or
R′″ and R³, together with the carbon atom to which they are attached, form an optionally substituted 3- to 7-membered saturated or unsaturated ring that optionally may have one or more heteroatoms selected from the group consisting of O, S, N or P;
except that R⁵ is OH, NH₂, or SH only when X or X* is C in Formulae I and III-VIII; [symbol]
is an optionally substituted carbocycle typically a 3-7 membered carbocyclic ring, or an optionally substituted heterocycle, typically a 3-7 membered heterocyclic ring having one or more O, S and/or N, that forms a spiro-nucleoside;
A′ is H, OH, C₁₋₄ alkyl, halo, azido, cyano, C₂₋₆ alkenyl, C₂-6 alkynyl, Br-vinyl, 2-Br-ethyl, —C(O)O(alkyl), —C(O)O(lower alkyl), —O(acyl), —O(lower acyl), —O(alkyl), —O(lower alkyl), —O(alkenyl), CF₃, NO₂, NH₂, —NH(lower alkyl), —NH(acyl), —N(lower alkyl)₂, or —N(acyl)₂; and
all tautomeric, enantiomeric and stereoisomeric forms thereof.

3. The method of claim 1 wherein the host is a mammal.

4. The method of claim 3 wherein the mammal is a human.

5. The method of claim 1, wherein the compound has a structure of Formula (i) or (ii);
A and B are H;
V is N;
Z is O; and
Y is NR′R″; or NR.

6. The method of claim 5, wherein R is H; C₁₋₁₀ cyclic or acyclic, branched or unbranched alkyl, optionally substituted with amino, carboxamido carboxylate or alkylamino; a 3-7 membered carbocycle or heterocycle; or a functional group that dissociates to provide the base where R is H, selected from the following structures:

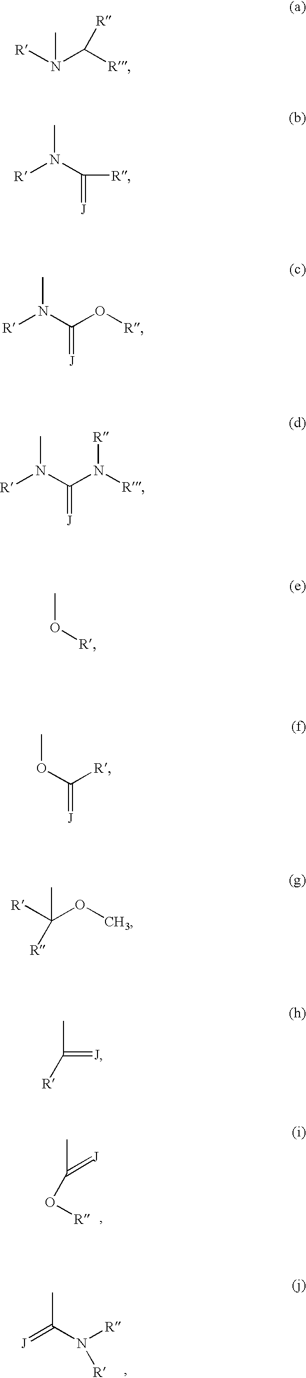

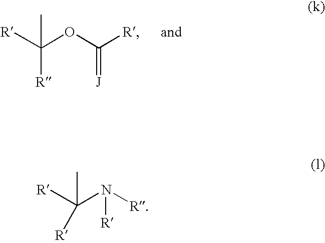

7. The method of claim 6, wherein each R′ and R″ independently is H; C₁₋₁₀ cyclic or acyclic, branched or unbranched alkyl, alkenyl, or alkynyl.

8. The method of claim 7, wherein R′″ is R′″ is H, OH, SH, halo, optionally substituted C₁₋₄ alkyl, optionally substituted C₂₋₄ alkenyl or C₂₋₄ alkynyl, where the optional substitutions on alkyl, alkenyl and/or alkynyl may be one or more halogen, hydroxy, amino, alkoxy, or alkylthio groups.

9. The method of claim 1 wherein the compound of Formula (i) or (ii) has the structure: 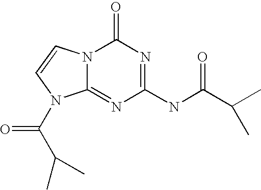
or a pharmaceutically acceptable salt or prodrug thereof.

10. The method of claim 1 wherein the compound of Formula (i) or (ii) has the structure: 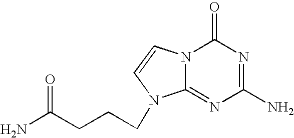
or a pharmaceutically acceptable salt or prodrug thereof.

11. A pharmaceutical composition comprising a compound of Formula (i) or (ii) having the structure: 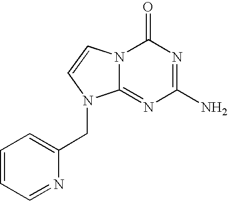
or a pharmaceutically acceptable salt or prodrug thereof, in combination with a pharmaceutically acceptable carrier, diluent or excipient.

12. A pharmaceutical composition comprising a compound of Formula (i) or (ii) having the structure: 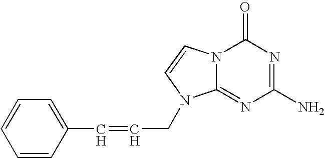
or a pharmaceutically acceptable salt or prodrug thereof, in combination with a pharmaceutically acceptable carrier, diluent or excipient.
